(19) United States
(12) Patent Application Publication
(10) Pub. No.: US 2026/0240762 A1
(43) Pub. Date: Aug. 20, 2026

(54) COMPOUND AND FORMULATION FOR SYSTEMIC THERAPY BY ORAL TRANSMUCOSAL ADMINISTRATION

(71) Applicant: CUROVIR AB, Kalmar (SE)

(72) Inventors: Jacob Westman, Järlåsa (SE); Anders Carlsson, Stockholm (SE)

(21) Appl. No.: 19/162,139

(22) PCT Filed: Mar. 7, 2024
(86) PCT No.: PCT/EP2024/055956

§ 371 Date: Sep. 4, 2025

(30) Foreign Application Priority Data
Mar. 9, 2023 (EP) 23161066.8

(51) Int. Cl. A61K 9/00; A61K 31/5025; A61K 31/519; A61K 31/53; A61P 31/14; C07D 487/04

(52) U.S. Cl. CPC A61K 9/006; A61K 31/5025; A61K 31/519; A61K 31/53; A61P 31/14; C07D 487/04

(57) ABSTRACT

Disclosed is a compound of formula (I)

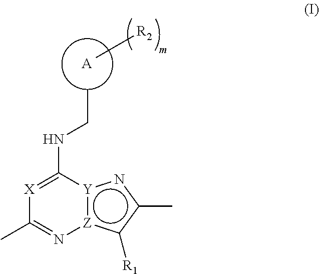

or a pharmaceutically acceptable salt thereof, for use in the systemic treatment of a virus infection or a disorder for which PI4K inhibition is beneficial, by oral transmucosal administration. A pharmaceutical formulation for oral transmucosal systemic administration of said compound.

### FIELD OF THE INVENTION

The present invention relates generally to certain aminosubstituted heteroaromatic compounds that are useful in the treatment of virus infections and disorders for which inhibition of phosphatidylinositol 4-kinase (PI4K) is beneficial. More particularly the invention relates to such compounds for use in the systemic treatment of virus infections and of disorders for which inhibition of PI4K is beneficial, by transmucosal administration. The invention furthermore relates to a pharmaceutical formulation for systemic transmucosal administration of such aminosubstituted heteroaromatic compounds.

### BACKGROUND OF THE INVENTION

PI4K has been shown to be involved in disorders of both infectious and non-infectious nature, including virus infections, cancers, autoimmune disorders and inflammatory disorders, and compounds capable of inhibiting PI4K have been shown to have a therapeutic utility in various types of diseases.

For example, international application No. PCT/US2020/023654 (WO 2020/191205) discloses PI4K inhibitors having usefulness in the treatment of various infectious diseases and non-infectious diseases. Infectious disease mentioned are infections viruses and other pathogens that utilize intracellular replication mechanisms, for examples viruses belonging to the Picornaviridae, Flaviviridae, Caliciviridae, Filoviridae, Hepeviridae or Coronavirinae families, as well as, for example, Plasmodium falciparium. Non-infectious diseases mentioned in said publication are various cancers for which PI4K inhibitors are useful to reduce cellular proliferation. International application No. PCT/US2017/044153 (WO 2018/022868) discloses PI4K inhibitors for the treatment of viral hepatitis, cancer, autoimmune disorders and inflammation. International application No. PCT/IB2017/050319 (WO 2017/125898) discloses PI4K inhibitors for the treatment of Cryptosporidium infections.

Certain aminosubstituted heteroaromatic compounds acting as PI4K inhibitors and antiviral agents have been previously described in international applications Nos. PCT/EP2015/051177 (WO 2015/110491), PCT/EP2016/063383 (WO 2016/206999), PCT/EP2018/058522 (WO 2018/185120), PCT/EP2019/072220 (WO 2020/074159) and PCT/EP2020/072847 (WO 2021/032611). A broad antiviral activity has been shown against viruses belonging to, for example, the Picornaviridae and Pneumoviridae families. The compounds have also been found to be potent inhibitors of PI4K, which renders them useful in the treatment of diseases for which inhibition of PI4K is therapeutically beneficial, such as pancreatitis, poliomyelitis, encephalitis, meningitis, sepsis, cancer, such as breast, prostate, ovarian and colorectal cancer, paralysis, cardiac diseases, such as myocarditis, diabetes, common cold, hand-foot-and-mouth disease, herpangina, pleurodynia, diarrhea, mucocutaneous lesions, respiratory illness, conjunctivitis, myositis, chronic fatigue syndrome, neuropsychiatric diseases, neurodegenerative diseases such as multiple sclerosis, Parkinson's disease, amyotrophic lateral sclerosis, Alzheimer's disease, and Huntington's disease, and inflammatory conditions.

In international application No. PCT/EP2019/072245 (WO 2020/074160), reference was made to prior art disclosing that inhibition of PI4KIIIβ (also referred to as PI4 KB) by oral administration of a PI4KIIIβ inhibitor is lethal to animals, and that therefore there is a concern that treatment by PI4KIIIβ inhibitors may have side effects in humans that may hamper their use in the therapy. WO 2020/074160 further mentioned the finding that in the enteral (e.g. oral) administration of certain PI4KIIIβ inhibitors the toxicity is mostly due to local intestinal side effects. It was therefore contemplated that the observed mortality in the in vivo tests could be due to local side effects of the PI4KIIIβ inhibitors in the intestines and, to overcome these unwanted effects, WO 2020/074160 proposed the use of prodrugs of the active compounds.

Transmucosal drug administration refers to the absorption of drug through the mucosal epithelium into the systemic circulation. In transmucosal administration, the intranasal, oral transmucosal and rectal routes are the major transmucosal routes. Transmucosal drug administration can reduce or completely bypass first-pass drug metabolism, avoid gastrointestinal degradation of the drug and provide rapid onset of action. However, not all the drugs can cross the mucosal epithelium effectively, due to the inherent properties thereof. The first barrier to the transmucosal drug delivery is presented by the mucus layer lining the mucosal epithelium. Mucus is composed mainly of water (about 95%) and mucin (about 2 to 5%), a high molecular weight glycoprotein that forms an entangled network which acts as a physical barrier to drug penetration, as well as creating an interactive barrier that limits drug diffusion. Once the drug molecule has passed the mucus layer, it may cross the underlying epithelium via a transcellular or paracellular route, both being passive transport processes driven by a local concentration gradient. For any given drug molecule, the capacity for crossing the epithelium via the two routes will depend on factors such as lipophilicity, molecular size, and state of ionization. In general, small lipophilic molecules that are non-ionised at the surrounding pH are favoured for transmucosal absorption, and in fact, many drugs do not exhibit the desirable physicochemical properties that would allow them to cross the mucosal epithelium effectively, and an efficient transport of the drug is difficult to achieve or hard to predict. Therefore, while in theory transmucosal administration is an attractive mode of administration, in practice it is still only of limited use.

Oral transmucosal administration (or delivery) refers to the systemic administration of drug through the mucous membrane of the oral cavity. Depending on the site of administration in the oral cavity, oral transmucosal delivery may be termed as a buccal route, where the drug is absorbed via the mucosa of the cheek and gum, and the sublingual route, where the drug is absorbed via the mucosa of the ventral surface of the tongue and the floor of the mouth, under the tongue.

There are different types of formulations for oral transmucosal administration, including buccal and sublingual tablets, lozenges to be placed between the cheeks and gums; liquid dosage forms e.g. for administrating in spray form, and oral (e.g. buccal or sublingual) films, which are essentially composed of polymeric matrices, usually based on hydrophilic polymers as main element, containing the active ingredient in the polymer matrix.

Transmucosal systemic delivery of a therapeutic agent could potentially offer various advantages, such as bypassing first-pass metabolism and avoidance of gastrointestinal side effects. Nonetheless, this administration mode is still only rarely used, due, in particular, to the difficulties linked to the barrier function of the mucosal membrane; the extent of permeation of any given compound is governed by factors such as lipid solubility, degree of ionization, molecular weight and size, and the success is generally unpredictable.

### SUMMARY OF THE INVENTION

A first aspect is a compound of formula (I)

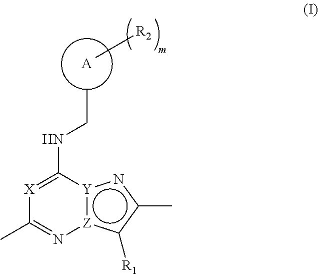

or a pharmaceutically acceptable salt thereof, wherein
X is CH, Y is C, and Z is N, or
X is CH, Y is N, and Z is C, or
X is N, Y is N, and Z is C;
R₁ is 3,4-dimethoxyphenyl or 1,3-dimethyl-1H-indazol-5-yl;
ring A is phenyl, or 5- or 6-membered heteroaryl;
mis 0, 1, 2 or 3;
each R₂ is independently selected from C₁-C₆ alkyl, halogen, R₃O, R₄R₅N, R₆R₇NS(O)₂, R₈R₉NC(O), R₁₀S(O)₂, R₁₁S(O)₂N(R₁₂), R₁₃C(O)O, R₁₄C(O)N(R₁₅), R₁₆OC(O), R₁₇C(O), R₁₈C(O)N(R₁₉)S(O)₂, R₂₀OC(O)N(R₂₁)S(O)₂, CN and —O—; and two R₃, attached to adjacent atoms of ring A, together with the atoms to which they are attached may form a 5- or 6-membered heterocyclic or carbocyclic ring;
each R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ is independently selected from H and C₁-C₆ alkyl; and
any alkyl is optionally substituted by one or more F;
for use in the systemic treatment of a virus infection or a disorder for which PI4K inhibition is beneficial, by oral transmucosal administration.

A further aspect is a pharmaceutical formulation for oral transmucosal systemic administration of a compound of formula (I) as defined herein above.

The pharmaceutical formulation preferably is for use in the treatment of a virus infection or a disorder for which PI4K inhibition is beneficial.

A further aspect is a method for the systemic treatment of a virus infection or a disorder for which PI4K inhibition is beneficial, by oral transmucosal administration of a therapeutically effective amount of a compound of formula (I) as defined herein, or a pharmaceutically acceptable salt thereof, to a mammal in need of such treatment.

A further aspect is the use of a compound of formula (I) as defined herein, or a pharmaceutically acceptable salt thereof, in the manufacture of a medicament allowing for oral transmucosal administration of said compound, for the systemic treatment of a virus infection or a disorder for which PI4K inhibition is beneficial.

A disorder for which PI4K inhibition is beneficial may be, for example, pancreatitis, poliomyelitis, encephalitis, meningitis, sepsis, cancer, such as breast, prostate, ovarian and colorectal cancer, paralysis, cardiac diseases, such as myocarditis, diabetes, common cold, hand-foot-and-mouth disease, herpangina, pleurodynia, diarrhea, mucocutaneous lesions, respiratory illness, conjunctivitis, myositis, chronic fatigue syndrome, neuropsychiatric diseases, neurodegenerative diseases, such as multiple sclerosis, Parkinson's disease, amyotrophic lateral sclerosis, Alzheimer's disease, and Huntington's disease, infectious diseases, and inflammatory conditions.

In preferred embodiments, the oral transmucosal administration is buccal transmucosal administration.

Further aspects and embodiments will become apparent from the following detailed description and are as defined in the claims.

### BRIEF DESCRIPTION OF THE DRAWINGS

FIG. 1 is a graph showing the mean concentration of a compound of formula (I) measured in blood from rabbits as a function of time after transmucosal administration of formulations according to Examples 1-4, and in blood from rabbits given the same compound by oral gavage.

FIG. 2 is a graph showing the mean concentration of a compound of formula (I) measured in blood from rabbits as a function of time after transmucosal administration of the film formulation according to Example 6, at a dosage of the active ingredient of either 7.5 mg/kg bodyweight or 15 mg/kg bodyweight.

### DETAILED DESCRIPTION OF THE INVENTION

Unless defined otherwise or clearly indicated by context, all technical and scientific terms and abbreviations used herein have the same meaning as commonly understood by one of ordinary skill in the art to which this disclosure belongs. However, definitions of some terms used herein will be given herein below.

As used herein, the singular forms “a,” “an” and “the” are intended to include the plural forms as well, unless the context clearly indicates otherwise. Thus, such references may be replaced with a reference to “one or more”, e.g., one, of the relevant component or integer. As used herein, all references to “one or more” of a particular component or integer will be understood to refer to from one to a plurality, e.g., two, three or four, of such components or integers. It will be understood that references to “one or more” of a particular component or integer will include a particular reference to one such integer. Also, as used herein, “and/or” refers to and encompasses any and all possible combinations of one or more of the associated listed items.

As used herein, the term “about,” when referring to a measurable value, such as an amount of a compound, dose, time, temperature, and the like, refers to variations of 20%, 10%, 5%, 1%, 0.5%, or even 0.1% of the specified amount. When a range is employed, e.g., a range from x to y, it is it meant that the measurable value is a range from about x to about y, or any range or value therein including x and y. It will be further understood that the terms “comprises” and/or “comprising,” when used in this specification, specify the presence of stated features, integers, steps, operations, elements, components and/or groups, but do not preclude the presence or addition of one or more other features, integers, steps, operations, elements, components, and/or groups thereof.

The terms “disease” and “disorder” are used synonymously herein, to refer to an abnormal condition that negatively affects the structure or function of all or part of a subject (e.g. a mammal), and that is not immediately due to any external injury. The disorder may be caused by external factors such as pathogens or by internal dysfunctions.

The term “effective” as used, for example, with respect to an amount effective to achieve an end, i.e., “therapeutically effective amount”, means the quantity of a component that is sufficient to yield an indicated therapeutic response without undue adverse side effects (such as toxicity, irritation, or allergic response) commensurate with a reasonable benefit/risk ratio when used in the manner of this disclosure. An effective amount may vary according to factors known in the art, such as the disorder state, age, sex, and weight of the human or animal being treated.

The term “excipient” refers to pharmaceutically acceptable chemicals, such as known to those of ordinary skill in the art of pharmacy to aid the administration of the medicinal agent. It is a compound that is useful in preparing a pharmaceutical composition, generally safe, non-toxic and neither biologically nor otherwise undesirable, and includes excipients that are acceptable for veterinary use as well as human pharmaceutical use. In the present context, exemplary excipients include plasticizers, solubilizers (solubilizing agents), colorants, taste-masking agents, antioxidants (antioxidation agents), preservatives, surfactants, saliva-stimulating agents, buffering agents, and penetration enhancers.

The term “film” refers to a type of dosage form that is distinctly different from pills, tablets, caplets, and capsules, and in which the dosage form is a thin strip of material.

The term “immediate release”, when used in respect of a transmucosal film of the invention, refers to a film releasing no less than 40% of its contents of active ingredient, preferably no less than 50%, more preferably no less than 60%, within 30 minutes of contact with a mucosal surface, e.g. an oromucosal surface.

The term “matrix” or “film matrix” refers to the matrix formed by the film forming polymer or mixture of polymers, and that contains the active ingredient within the film dosage form.

The term “mucoadhesive”, such as in “mucoadhesive agent” refers to the characteristic of being able to adhere to a mucosal surface (via interaction with the mucus covering the epithelia) e.g. the mucosal surface in the mouth of a human.

“Optional” or “optionally” means that the subsequently described event or circumstance may or may not occur, and that the description includes instances where the event or circumstance occurs and instances where it does not.

A “patient” as referred to herein may be an animal, e.g. a non-human mammal or a bird, or a human.

The term “mammal” refers to a human or any mammalian animal, e.g. a primate, a farm animal, a pet animal, or a laboratory animal. Examples of such animals are monkeys, cows, sheep, goats, horses, pigs, dogs, cats, rabbits, mice, rats etc. Preferably, the mammal is a human. In some embodiments, however, the mammal is an animal, e.g. a farm animal, such as a cow, sheep, goat, horse, or pig. In some other embodiments, the animal is a pet, e.g. a dog, a cat, or a rabbit.

A “bird” as referred to herein may be, for example, a poultry, a cage bird or a pet bird.

The term “pharmaceutically acceptable” means being useful in preparing a pharmaceutical composition that is generally safe, non-toxic and neither biologically nor otherwise undesirable and includes being useful for veterinary use as well as human pharmaceutical use.

The term “PI4K” refers to an enzyme belonging to the family of PI4-kinases. The PI4K family includes four isoforms, viz. phosphatidylinositol 4-kinase alpha (PI4KA), phosphatidylinositol 4-kinase beta (PI4 KB), phosphatidylinositol 4-kinase 2-alpha (PI4K2A), and phosphatidylinositol 4-kinase 2-beta (PI4K2B), differing in subcellular localization and being involved in the synthesis of distinct phosphatidylinositol 4-phosphate (PI4P) pools.

A “medicament suitable for oral transmucosal administration” refers to a medicament having a form that makes it suitable for administration through oral mucosa.

The term “plasticizer” refers to refers to a component that reduces the glass-transition temperature of the film forming polymers and, thereby, increases the flexibility, enhances elasticity and reduces brittleness of the film.

The term “preservative”, as used herein, refers to an additive which inhibits both microbial growth and kills microorganisms that contaminate a formulation exposed to the surroundings.

The term “respiratory tract” as used herein refers to the respiratory organs of a patient (bird or mammal) and includes the upper respiratory tract and lower respiratory tract.

The “upper respiratory tract” includes the nose and nasal passages, paranasal sinuses, the pharynx, and the portion of the larynx above the vocal folds.

The “lower respiratory tract” includes the trachea, bronchi, the bronchioles and lungs.

The term “solubilizing agent” (or alternatively “solubilizer”) refers to a compound that, when added to a solvent phase or formulation, is capable of increasing the solubility of another compound in said solvent phase or formulation.

The term “solubilizing effective amount” of a substance (“solubilizer”) within a formulation refers to an amount of the substance sufficient to solubilize another component of the composition, or sufficient to improve solubility of the component.

The term “surfactant” refers to an organic chemical compound capable of lowering the surface tension (or interfacial tension) between two liquids, between a gas and a liquid, or between a liquid and a solid. A surfactant is an amphiphilic compound, i.e. a compound that contains a hydrophobic moiety (“the hydrophobic tail”) as well as a hydrophilic moiety (the “hydrophilic head” or “polar head”). Most commonly, surfactants are classified according to the hydrophilic head. A “non-ionic surfactant” has no electrically charged groups in its head; the hydrophilic head of a “cationic surfactant” carries a net positive electrical charge, and the hydrophilic head of an “anionic surfactant” carries a net negative electrical charge.

The term “systemic” as used herein means something that generally affects the whole body of a subject, e.g. a mammal patient, for example, as opposed to “local”.

“Systemic treatment” refers to treatment whereby an active agent is delivered to organs of the body of a subject, e.g. a mammal patient, via the circulating blood stream of the subject.

“Therapeutically effective amount” refers to an amount of a compound that, when administered to a patient for treating a disease, is sufficient to effect such treatment of the disease. The “therapeutically effective amount” will vary depending on the compound, the disease and its severity as well as the age, weight, etc., of the patient to be treated.

The term “transmucosal,” as used herein, refers to administration via a mucosal membrane. Examples include, but are not limited to, oral (e.g. buccal or sublingual), nasal, vaginal, and rectal. When the administration is through the oral mucosa and may then be referred to as being “oral transmucosal” or “oromucosal”.

The term “treating” (or “treatment”) of a disease or disorder may refer to ameliorating the disease or disorder (i.e., arresting or reducing the development of the disease or at least one of the clinical symptoms thereof), and/or ameliorating at least one physical parameter, which may not be discernible by the patient. The term also may refer to inhibiting the disease or disorder, either physically, (e.g., stabilization of a discernible symptom), physiologically, (e.g., stabilization of a physical parameter), or both.

As used herein, “treating” encompasses, e.g., inducing inhibition, regression, or stasis of a disease, disorder or condition, or ameliorating or alleviating a symptom of a disease, disorder or condition. “Ameliorating” or “alleviating” a condition or state as used herein shall mean to relieve or lessen the symptoms of that condition or state. “Inhibition” of disease progression or disease complication in a subject as used herein means preventing or reducing the disease progression and/or disease complication in the subject.

The term “unit dose” as used herein is the amount of the inventive formulation to be administered to the subject in a single administration, or the amount active ingredient contained in said amount of the inventive formulation. The unit dose disclosed herein can be administered one or more times, for example, periodically on a daily basis, weekly basis or monthly basis.

A “virus infection” (which may also be referred to as a viral infection) refers to an infection by a virus, in a mammal.

An “RNA virus infection” refers to a virus infection wherein the virus has RNA (ribonucleic acid) as its genetic material.

A “non-enveloped single-stranded (+) RNA virus infection” refers to an infection by a non-enveloped single-stranded (+) RNA virus.

A “non-enveloped virus” is a virus lacking virus envelope.

A “single-stranded (+) RNA virus”, or +ssRNA virus, is a virus having genetic material which is single-stranded RNA and which RNA can be immediately translated to viral protein by the cell infected by the virus.

An “enveloped single-stranded (−) RNA virus infection” refers to an infection by an enveloped single-stranded (−) RNA virus.

An “enveloped virus” is a virus having a virus envelope.

A “single-stranded (−) RNA virus” is a virus having genetic material which is single-stranded, negative-sense RNA, which RNA must first be converted to positive-stranded RNA before being translated to viral protein by the cell infected by the virus.

A “Pneumoviridae viral infection” refers to an infection by a virus belonging to the taxonomic family Pneumoviridae, in a mammal. The viral family Pneumoviridae contains the two genera Metapneumovirus and Orthopneumovirus.

An “orthopneumoviral infection” refers to an infection by a virus belonging to the Orthopneumovirus genus.

A “metapneumoviral infection” refers to an infection by a virus belonging to the Metapneumovirus genus.

Unless otherwise stated or indicated, the term “halogen” (or “halo”) refers to fluorine (F), chlorine (Cl), or bromine (Br).

A moiety of the type RO is a moiety of formula

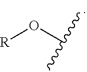

A moiety of the type RR′NC(O) is a moiety of formula

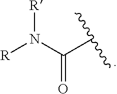

A moiety of the type RC(O)N(R′) is a moiety of formula

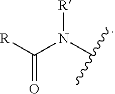

A moiety of the type RS(O)₂ is a moiety of formula

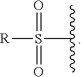

A moiety of the type RS(O)₂N(R′) is a moiety of formula

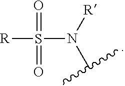

A moiety of the type RR′NS(O)₂ is a moiety of formula

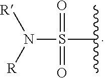

A moiety of the type RR′N is a moiety of formula

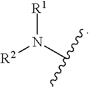

A moiety of the type RC(O) is a moiety of formula

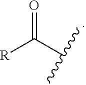

A moiety of the type ROC(O) is a moiety of formula

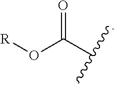

A moiety of the type RC(O)O is a moiety of formula

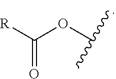

A moiety of the type RC(O)N(R′)S(O)₂, is a moiety of formula

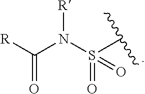

A moiety of the type and ROC(O)N(R′)S(O)₂ is a moiety of formula

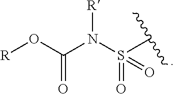

A moiety of the type —O⁻ is a covalently bound oxygen anion, i.e. a moiety of formula

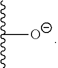 [SMILES: *[O-]]

A moiety CN may also be referred to as “cyano” and is a moiety of formula

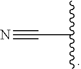 [SMILES: *C#N]

The term “heterocyclic ring” refers to a saturated or unsaturated, aromatic or non-aromatic cyclic moiety containing not only carbon atoms, but also at least one other atom in the ring, e.g. selected from nitrogen (N), sulphur (S) and oxygen (O). When aromatic, the heterocyclyl also may be referred to as “heteroaryl”, which refers to an aromatic ring containing at least one ring heteroatom, such as furyl, isoxazolyl, isothiazolyl, imidazolyl, pyridyl, pyrrolyl, pyrazolyl, pyrimidinyl, pyridazinyl, pyrazinyl, oxadiazolyl, oxazolyl, thienyl, thiadiazolyl, thiazolyl, triazolyl, and tetrazolyl.

The term “aromatic”, as used herein, refers to an unsaturated cyclic moiety that has an aromatic character, while the term “non-aromatic”, as used herein, refers to a cyclic moiety, that may be saturated or unsaturated, e.g. polyunsaturated, but that does not have an aromatic character.

The term “benzyl” refers to a moiety of formula C₆H₅CH₂—, i.e.;

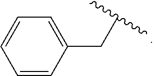 [SMILES: *Cc1ccccc1]

The term “pyridyl” (or pyridinyl) refers to a moiety of formula

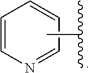

The term “3-pyridyl” (or “pyridin-3-yl”) refers to a moiety of formula

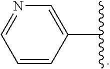 [SMILES: *c1cccnc1]

A 3-pyridyl substituted with a moiety R₂ in 6-position (e.g. 6-methyl-3-pyridyl) is a compound of formula

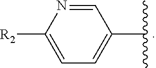

The term “4-pyridyl” (or pyridin-4-yl) refers to a moiety of formula

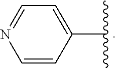 [SMILES: *c1ccncc1]

The term “thienyl” refers to a moiety of formula

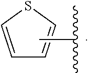

The term “2-thienyl” refers to a moiety of formula

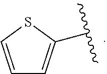 [SMILES: *c1cccs1]

The term “furyl” refers to a moiety of formula

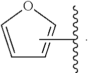

The term “2-furyl” refers to a moiety of formula

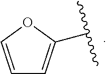 [SMILES: *c1ccco1]

In a compound of formula (I), X is CH, Y is C, and Z is N; or X is CH, Y is N, and Z is C; or X is N, Y is N, and Z is C.

In some embodiments, X is CH, Y is C, and Z is N; or X is CH, Y is N, and Z is C; i.e. the compound may be represented by formula (Ia)

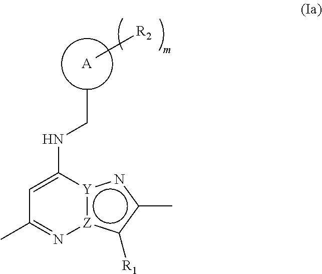

wherein Y, Z, R₁, R₂, ring A and m are as defined herein.

In some embodiments, X is CH, Y is N, and Z is C; or X is N, Y is N, and Z is C. In some embodiments, X is CH, Y is C, and Z is N; or X is N, Y is N, and Z is C.

In some embodiments, X is CH, Y is N, and Z is C. In these embodiments, the compound may be represented by formula (Ib)

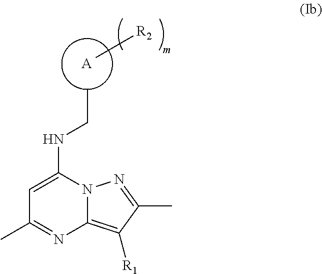

wherein R₁, R₂, ring A and m are as defined herein.

In some other embodiments, X is CH, Y is C, and Z is N. In these embodiments, the compound may be represented by formula (Ic)

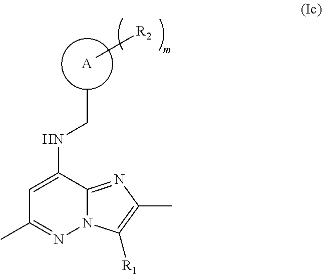

wherein R₁, R₂, ring A and m are as defined herein.

In some embodiments, X is N, Y is N, and Z is C. In these embodiments, the compound may be represented by formula (Id)

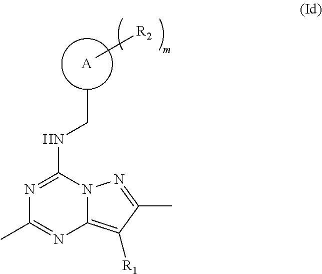

wherein R₁, R₂, ring A and m are as defined herein.

The moiety R₁ is 3,4-dimethoxyphenyl or 1,3-dimethyl-1H-indazol-5-yl. In some embodiments, R₁ is 3,4-dimethoxyphenyl, i.e. the compound may be represented by formula (Ie)

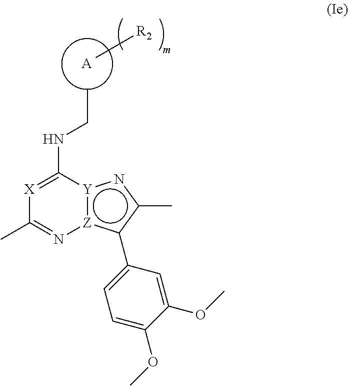

wherein X, Y, Z, R₂, ring A and m are as defined herein.

In some embodiments, R₁ is 1,3-dimethyl-1H-indazol-5-yl, i.e. the compound may be represented by formula (If)

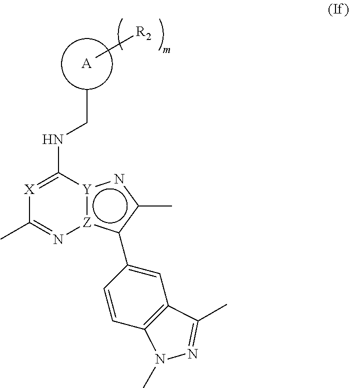

wherein X, Y, Z, R₂, ring A and m are as defined herein.

The compound of formula (I) comprises a moiety of formula (II)

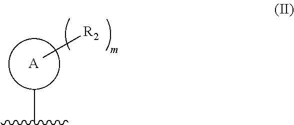

wherein R₂, ring A and m are as defined herein.

In the moiety of formula (II), ring A is phenyl or 5- or 6-membered heteroaryl. The heteroaryl preferably has one or more heteroatoms selected from O, S, and N. In some embodiments, ring A is 5- or 6-membered heteroaryl. In some other embodiments, ring A is phenyl or 6-membered heteroaryl. In still other embodiments, ring A is phenyl. In still other embodiments, ring A is phenyl or 5-membered heteroaryl. In some embodiments, ring A is 5-membered heteroaryl. In some other embodiments, ring A is 6-membered heteroaryl.

When ring A is 5- or 6-membered heteroaryl, said heteroaryl contains one or more heteroatoms. For example, said heteroaryl may contain 1, 2, 3, or 4 heteroatoms. In some embodiments, said heteroaryl contains 1, 2 or 3 heteroatoms. In some other embodiments, said heteroaryl contains or 1 or 2 heteroatoms. In still other embodiments, said heteroaryl contains 1 heteroatom.

Each heteroatom is independently selected from N, O and S. In some embodiments, each heteroatom is independently selected from N and O. In still other embodiments each heteroatom is independently selected from N and S. In some particular embodiments, at least one heteroatom is N, e.g. each heteroatom is N. In some embodiments, the heteroaryl contains one heteroatom, which is N. In some other embodiments, the heteroaryl contains one heteroatom, which is O. In some other embodiments, the heteroaryl contains one heteroatom, which is S.

In some embodiments, when ring A is 5-membered heteroaryl, said heteroaryl contains one heteroatom selected from O and S, and optionally one or more nitrogen atoms, e.g. 1-3 N, or 1 or 2 N, e.g. 1 N. In some embodiments, said heteroaryl is thienyl or furyl, e.g. 2-thienyl or 2-furyl. In some embodiments, when ring A is 5-membered heteroaryl, said heteroaryl more particularly is thienyl, e.g. 2-thienyl.

In some embodiments, when ring A is 6-membered heteroaryl, each heteroatom of said heteroaryl is N. For example, in some embodiments, when ring A is 6-membered heteroaryl, said heteroaryl is pyridyl, e.g. 3-pyridyl or 4-pyridyl, in particular 4-pyridyl.

In some embodiments, when ring A is 5- or 6-membered heteroaryl, said heteroaryl is selected from thienyl, furyl, pyrrolyl, imidazolyl, pyrazolyl, thiazolyl, isothiazolyl, oxazolyl, isoxazolyl, triazolyl, tetrazolyl, oxadiazolyl, thiadiazolyl, pyridyl, pyrazinyl, pyrimidinyl, and pyridazinyl.

In some embodiments, when ring A is phenyl or 6-membered heteroaryl, the moiety of formula (II) more particularly is a moiety of formula (IIa)

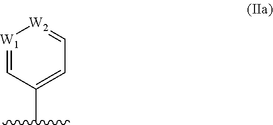

wherein W₁ and W₂ are independently selected from N, CH and CR₂. In such embodiments, the compound may be represented by formula (Ig)

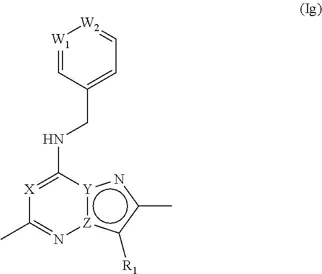

wherein X, Y, Z, R₁, W₁ and W₂ are as defined herein.

In some embodiments, one of W₁ and W₂ is different from N. In some embodiments, at least one of W₁ and W₂ is N. In some embodiments, at least one of W₁ and W₂ is different from CH. In some embodiments, at least one of W₁ and W₂ is different from CR₂. In some embodiments, W₁ and W₂ are different from each other, i.e. W₁ and W₂ are not both N, not both CH and not both CR₂. In some particular embodiments, W₁ is as defined herein and W₂ is N or CR₂, in particular N.

In some further embodiments, the moiety of formula (II) is selected from a moiety comprising 5-membered heteroaryl, e.g. unsubstituted (m is 0) 5-membered heteroaryl, and a ring of formula (IIa) as defined herein; e.g. from unsubstituted 2-thienyl and a ring of formula (IIa) wherein W₁ is CH and W₂ is CR₂, or W₁ is CH and W₂ is N, or W₁ is N and W₂ is CR₂. In some further embodiments, the moiety of formula (II) is selected from unsubstituted 2-thienyl and a ring of formula (IIa) wherein W₁ is CH and W₂ is N, or W₁ is N and W₂ is CR₂.

In some particular embodiments, the moiety of formula (IIa) is selected from

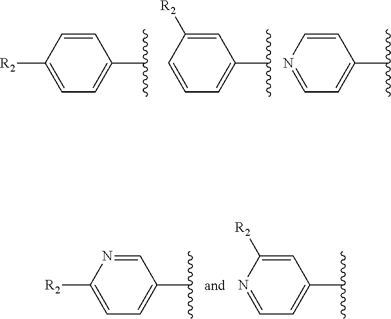

wherein R₂ is as defined herein.

In some embodiments, W₁ is CH or CR₂, and W₂ is N; or W₁ is N, and W₂ is CH₃; i.e. the moiety of formula (IIa) is selected from

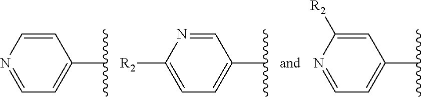

wherein R₂ is as defined herein.

In some further embodiments, W₁ is CH or CR₂, and W₂ is N, i.e. the moiety of formula (IIa) is selected from

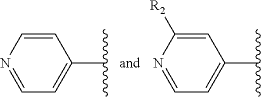

wherein R₂ is as defined herein, e.g. R₂ is selected from C₁-C₆ alkyl (including C₁-C₆ alkyl substituted by one or more fluoro), halogen, R₃O, and CN.

In some further embodiments, W₁ is CR₂ and W₂ is N, i.e. the moiety of formula (IIa) is

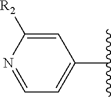

wherein R₂ is as defined herein, e.g. R₂ is selected from C₁-C₆ alkyl (including C₁-C₆ alkyl substituted by one or more fluoro), halogen, R₃O, and CN; e.g. R₂ is selected from C₁-C₆ alkyl.

In still some further embodiments, W₁ is CH and W₂ is N.

In still some further embodiments, W₁ is CH or N, and W₂ is CR₂; or W₁ is CH and W₂ is N; i.e. the moiety of formula (IIa) is selected from

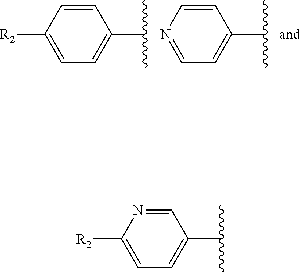

wherein R₂ is as defined herein.

In some further embodiments, W₁ is CH and W₂ is N; or W₁ is N and W₂ is CR₂, i.e. the moiety of formula (IIa) is selected from

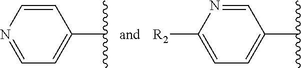

wherein R₂ is as defined herein.

In some further embodiments, W₁ is CH and W₂ is CR₂; or W₁ is N and W₂ is CR₂, i.e. the moiety of formula (IIa) is a moiety selected from

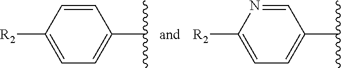

wherein R₂ is as defined herein.

In some further embodiments, W₁ is CH and W₂ is CR₂; or W₁ is CR₂ and W₂ is CH, i.e. the moiety of formula (IIa) is a moiety selected from

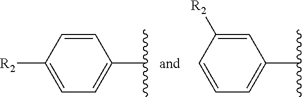

wherein R₂ is as defined herein.

In some embodiments, W₁ is CH and W₂ is CR₂. In some further embodiments, W₁ is CR₂ and W₂ is CH.

In a compound of formula (I), m is an integer 0, 1, 2 or 3 that denotes the number of substituents on ring A, i.e. ring A may be unsubstituted (m is 0) or substituted by 1, 2 or 3 moieties R₂ (m is 1, 2 or 3). In some embodiments, m is 0, 1 or 2. In some embodiments, m is 0 or 1. In some other embodiments, m is 0. In still other embodiments, m is 1, 2 or 3; or 1 or 2; in particular m is 1.

In some embodiments, when ring A is phenyl, m is different from 0. Thus, in some embodiments, when ring A is phenyl, m is as indicated herein above, but is not 0, e.g. m is 1 or 2, and when ring A is 5- or 6-membered heteroaryl, m is as indicated herein above, e.g. m is 0 or 1. In some other embodiments, when ring A is phenyl, m is 1, and when ring A is 5- or 6-membered heteroaryl, m is 0 or 1. In still further embodiments, when ring A is phenyl, m is 1, and when ring A is 5- or 6-membered heteroaryl, m is 0. In some further embodiments, when ring A is phenyl, m is 1, when ring A is 5-membered heteroaryl, m is 0, and when ring A is 6-membered heteroaryl, m is 0 or 1.

In some embodiments, ring A is phenyl and m is 1 or 2, or ring A is 5- or 6-membered heteroaryl and m is 0, 1 or 2. In some other embodiments, ring A is phenyl and m is 1, or ring A is 5- or 6-membered heteroaryl, and m is 0 or 1. In still further embodiments, ring A is phenyl, and m is 1, or ring A is 5- or 6-membered heteroaryl, and m is 0. In some further embodiments, ring A is phenyl, and m is 1, or ring A is 5-membered heteroaryl, and m is 0, or ring A is 6-membered heteroaryl, and m is 0 or 1.

In some embodiments, ring A is phenyl, and m is 0, 1 or 2, in particular 1 or 2. In some embodiments, ring A is phenyl, and m is 1.

In some embodiments, ring A is 5- or 6-membered heteroaryl, and m is 0 or 1. In some embodiments, ring A is 5- or 6-membered heteroaryl, and m is 0. In some embodiments, ring A is 5-membered heteroaryl, and m is 0. In some embodiments, ring A is 6-membered heteroaryl, and m is 0 or 1. In some embodiments, ring A is 6-membered heteroaryl, and m is 1. In some embodiments, ring A is 6-membered heteroaryl, and m is 0.

In some embodiments, when ring A is 3-pyridyl, m is 1 or 2, in particular 1, and one R₂ is attached in 6-position on the pyridyl ring.

It should be realized that, unless mutually exclusive, and whether illustrated herein or not, any combinations of the embodiments as illustrated by formulas (Ia) to (Ig) are contemplated within the scope of formula (I). For example, in some embodiments, a compound of formula (Ia) (e.g. a compound of formula (Ib), or a compound of formula (Ic)), also is a compound of formula (Ie). In some further embodiments, a compound of formula (Ia) (e.g. a compound of formula (Ib), or a compound of formula (Ic)), also is a compound of formula (If).

In some embodiments, a compound of formula (Ia) also is a compound of formula (Ig), i.e. a compound that may be represented by formula (Ih)

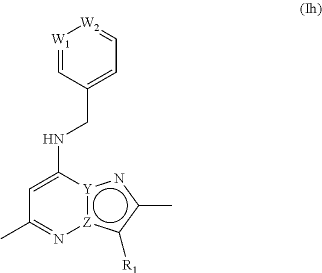

wherein Y, Z, R₁, W₁ and W₂ are as defined herein.

In some embodiments, a compound of formula (Ih) also is a compound of formula (Ie), i.e. the compound is one as represented by formula (Ij)

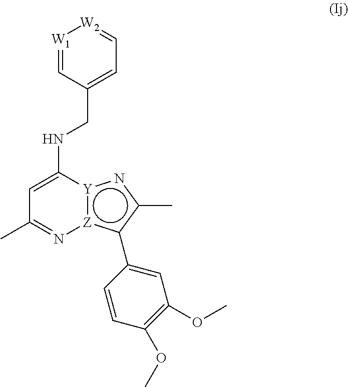

wherein Y, Z, W₁ and W₂ are as defined herein.

In still further embodiments, a compound of formula (Ij), also is a compound of formula (Ib), i.e. a compound as represented by formula (Ik)

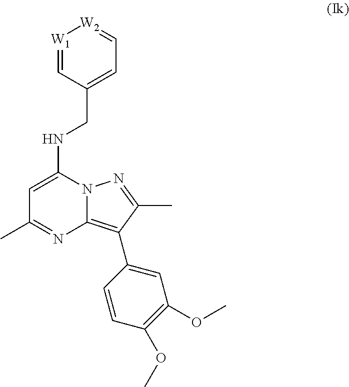

wherein W₁ and W₂ are as defined herein.

In some other embodiments, a compound of formula (Ih) also is a compound of formula (If), i.e. the compound is one as represented by formula (Im)

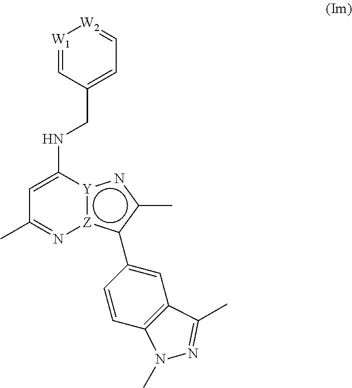

wherein Y, Z, W₁ and W₂ are as defined herein.

In still further embodiments, a compound of formula (Im), also is a compound of formula (Ib), i.e. a compound as represented by formula (In)

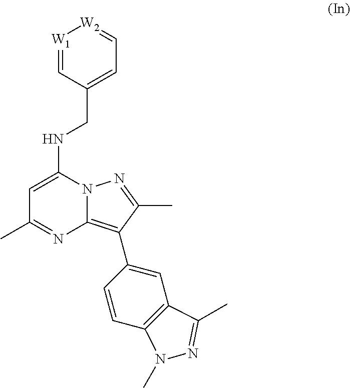

wherein W₁ and W₂ are as defined herein.

In some further embodiments, the compound of formula (I) is a compound of formula (Io)

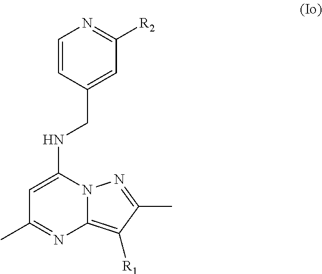

wherein R₁ and R₂ are as defined herein.

In some embodiments, the compound of formula (I) is a compound of formula (Ip)

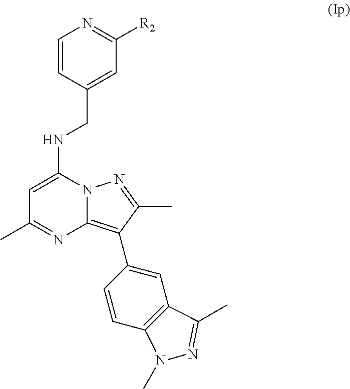

wherein R₂ is as defined herein.

It should be realized that any reference made herein to a compound of formula (I) implicitly also is a reference to a compound of any of the embodiments of such a compound, e.g. as illustrated in any one of the formulas (Ia) to (Ip), unless otherwise indicated or apparent from the context.

In a compound of formula (I), each R₂ is independently selected from C₁-C₆ alkyl, halogen, R₃O, R₄R₅N, R₆R₇NS(O)₂, R₈R₉NC(O), R₁₀S(O)₂, R₁₁S(O)₂N(R₁₂), R₁₃C(O)O, R₁₄C(O)N(R₁₅), R₁₆C(O), R₁₇C(O), R₁₈C(O)N(R₁₉)S(O)₂, R₂₀OC(O)N(R₂₁)S(O)₂, CN and —O—; and two R₂, attached to adjacent atoms of ring A, together with the atoms to which they are attached may form a 5- or 6-membered heterocyclic or carbocyclic ring.

In some embodiments, each R₂ is independently selected from C₁-C₆ alkyl, halogen, R₃O, R₄R₅N, R₆R₇NS(O)₂, R₁₀S(O)₂, R₁₁S(O)₂N(R₁₂), R₁₄C(O)N(R₁₅), R₁₇C(O), R₁₈C(O)N(R₁₉)S(O)₂, R₂₀OC(O)N(R₂₁)S(O)₂, CN and —O—; and two R₂, attached to adjacent atoms of ring A, together with the atoms to which they are attached may form a 5- or 6-membered heterocyclic or carbocyclic ring.

In some embodiments, each R₂ is independently selected from C₁-C₆ alkyl, halogen, R₃O, R₆R₇NS(O)₂, R₁₀S(O)₂, R₁₁S(O)₂N(R₁₂), and R₁₄C(O)N(R₁₅), and two R₂, attached to adjacent atoms of ring A, together with the atoms to which they are attached may form a 5- or 6-membered heterocyclic or carbocyclic ring.

In some embodiments, each R₂ is independently selected from C₁-C₆ alkyl, halogen, R₃O, R₆R₇NS(O)₂, R₁₀S(O)₂, and R₁₁S(O)₂N(R₁₂), and two R₂, attached to adjacent atoms of ring A, together which they are attached may form a 5- or 6-membered heterocyclic or carbocyclic ring.

In some embodiments, each R₂ is independently selected from C₁-C₆ alkyl, halogen, and R₃O, and two R₂, attached to adjacent atoms of ring A, together which they are attached may form a 5- or 6-membered heterocyclic or carbocyclic ring.

In some embodiments, each R₂ is independently selected from C₁-C₆ alkyl and R₃O, and two R₂, attached to adjacent atoms of ring A, together which they are attached may form a 5- or 6-membered heterocyclic or carbocyclic ring.

In some embodiments, each R₂ is independently selected from C₁-C₆ alkyl and halogen, and two R₂, attached to adjacent atoms of ring A, together which they are attached may form a 5- or 6-membered heterocyclic or carbocyclic ring.

In some further embodiments, each R₂ is independently selected from C₁-C₆ alkyl, halogen, R₃O, R₄R₅N, R₆R₇NS(O)₂, R₁₀S(O)₂, R₁₁S(O)₂N(R₁₂), R₁₄C(O)N(R₁₅), R₁₇C(O), R₁₈C(O)N(R₁₉)S(O)₂, and R₂₀OC(O)N(R₂₁)S(O)₂, and two R₂, attached to adjacent atoms of ring A, together with the atoms to which they are attached may form a 5- or 6-membered heterocyclic or carbocyclic ring.

In some further embodiments, each R₂ is independently selected from C₁-C₆ alkyl, halogen, R₃O, R₄R₅N, R₆R₇NS(O)₂, R₁₀S(O)₂, and R₁₁S(O)₂N(R₁₂), and two R₂, attached to adjacent atoms of ring A, together with the atoms to which they are attached may form a 5- or 6-membered heterocyclic or carbocyclic ring.

In some further embodiments, each R₂ is independently selected from C₁-C₆ alkyl, halogen, R₃O, R₆R₇NS(O)₂, R₁₀S(O)₂, and R₁₁S(O)₂N(R₁₂), and two R₂, attached to adjacent atoms of ring A, together with the atoms to which they are attached may form a 5- or 6-membered heterocyclic or carbocyclic ring.

In some embodiments, each R₂ is independently selected from C₁-C₆ alkyl, halogen, R₃O, R₄R₅N, R₆R₇NS(O)₂, R₈R₉NC(O), R₁₀S(O)₂, R₁₁S(O)₂N(R₁₂), R₁₃C(O)O, R₁₄C(O)N(R₁₅), R₁₆OC(O), R₁₇C(O), R₁₈C(O)N(R₁₉)S(O)₂, R₂₀OC(O)N(R₂₁)S(O)₂, CN and —O⁻. In some embodiments, each R₂ is independently selected from C₁-C₆ alkyl, halogen, R₃O, R₄R₅N, R₆R₇NS(O)₂, R₁₀S(O)₂, R₁₁S(O)₂N(R₁₂), R₁₄C(O)N(R₁₅), R₁₇C(O), R₁₈C(O)N(R₁₉)S(O)₂, R₂₀OC(O)N(R₂₁)S(O)₂, CN and —O⁻. In some embodiments, each R₂ is independently selected from C₁-C₆ alkyl, halogen, R₃O, R₆R₇NS(O)₂, R₁₀S(O)₂, R₁₁S(O)₂N(R₁₂), and R₁₄C(O)N(R₁₅). In some embodiments, each R₂ is independently selected from C₁-C₆ alkyl, halogen, R₃O, R₆R₇NS(O)₂, R₁₀S(O)₂, and R₁₁S(O)₂N(R₁₂). In some embodiments, each R₂ is independently selected from C₁-C₆ alkyl, R₆R₇NS(O)₂, R₁₀S(O)₂, and R₁₁S(O)₂N(R₁₂). In some embodiments, each R₂ is independently selected from C₁-C₆ alkyl, R₆R₇NS(O)₂, and R₁₀S(O)₂. In some embodiments, one R₂ is independently selected from R₆R₇NS(O)₂, and R₁₀S(O)₂. In some embodiments, one R₂ is R₆R₇NS(O)₂. In some embodiments, one R₂ is R₁₀S(O)₂. In some embodiments, each R₂ is independently selected from C₁-C₆ alkyl, halogen, and R₃O. In some embodiments, each R₂ is independently selected from C₁-C₆ alkyl and R₃O. In some embodiments, each R₂ is independently selected from C₁-C₆ alkyl and halogen. In some embodiments, each R₂ is independently selected from C₁-C₆ alkyl. In some embodiments, each R₂ is independently selected from halogen.

In some further embodiments, each R₂ is independently selected from C₁-C₆ alkyl, halogen, R₃O, R₄R₅N, R₆R₇NS(O)₂, R₁₀S(O)₂, R₁₁S(O)₂N(R₁₂), R₁₄C(O)N(R₁₅), R₁₇C(O), R₁₈C(O)N(R₁₉)S(O)₂, and R₂₀OC(O)N(R₂₁)S(O)₂.

In some further embodiments, each R₂ is independently selected from C₁-C₆ alkyl, halogen, R₃O, R₄R₅N, R₆R₇NS(O)₂, R₁₀S(O)₂, and R₁₁S(O)₂N(R₁₂).

In some further embodiments, each R₂ is independently selected from C₁-C₆ alkyl, halogen, R₃O, R₆R₇NS(O)₂, R₁₀S(O)₂, and R₁₁S(O)₂N(R₁₂). In some further embodiments, each R₂ is independently selected from C₁-C₆ alkyl, R₆R₇NS(O)₂, and R₁₀S(O)₂.

In some further embodiments, each R₂ is independently selected from C₁-C₆ alkyl, halogen, R₃O, and CN.

When R₂ is C₁-C₆ alkyl, it more particularly may be C₁-C₅ alkyl, or C₁-C₄ alkyl, or C₁-C₃ alkyl, or C₁-C₂ alkyl, e.g. methyl.

When R₂ is halogen, it e.g. may be selected from F, Cl, and Br, more particularly from F and Cl. In some embodiments, when R₂ is halogen, said halogen is F.

When R₂ comprises a moiety R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁, any such moiety is independently selected from H and C₁-C₆ alkyl. In some embodiments, any such moiety is independently selected from H and C₁-C₅ alkyl, or from H and C₁-C₄ alkyl, or from H and C₁-C₃ alkyl, or from H and C₁-C₂ alkyl, e.g. from H and methyl.

When any one of R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁, is a moiety selected from C₁-C₆ alkyl, such moiety more particularly may be selected from C₁-C₅ alkyl, or from C₁-C₄ alkyl, or from C₁-C₃ alkyl, or from C₁-C₂ alkyl, e.g. it may be methyl.

In some embodiments, each R₁₂, R₁₅, R₁₉, and R₂₁, when present, is H.

In some embodiments, each R₃, R₄, R₅, R₆, R₇, R₈, R₉, R₁₀, R₁₁, R₁₃, R₁₄, R₁₆, R₁₇, R₁₈, and R₂₀, when present, is selected from C₁-C₆ alkyl; and each R₁₂, R₁₅, R₁₉, and R₂₁, when present, is H.

In some embodiments, each R₃, R₄, R₅, R₆, R₇, R₈, R₉, R₁₃, and R₁₆, when present, is selected from H and C₁-C₆ alkyl; each R₁₀, R₁₁, R₁₄, R₁₇, R₁₈, and R₂₀, when present, is selected from C₁-C₆ alkyl; and each R₁₂, R₁₅, R₁₉, and R₂₁, when present, is H.

Any alkyl present in R₂, or as a moiety R₂, may optionally be substituted by one or more F, i.e. any alkyl may be or comprise a moiety selected from CF₃—, CF₂H—, CFH₂—, —CF₂—, —CFH—, and —CF<. In some embodiments, no such fluoro substitution is present in any alkyl group present in or as a moiety R₂.

In some embodiments, when m is at least 2, e.g. m is 2, two R₂ attached to adjacent atoms of the ring A, together with the atoms to which they are attached, form a 5- or 6-membered heterocyclic or carbocyclic ring. In some embodiments, the ring is carbocyclic. In some other embodiments, the ring is heterocyclic. In some embodiments, the ring is carbocyclic or 5-membered and heterocyclic.

In some embodiments, when two R₂ attached to adjacent atoms of the ring A form, together with the atoms to which they are attached, a 5- or 6-membered heterocyclic ring, said ring is a 5-membered heterocyclic ring, e.g. 1,3-dioxole or 1,3-dioxolane. In some embodiments, when two R₂ attached to adjacent atoms of the ring A, together with the atoms to which they are attached, form a carbocyclic ring, said ring is a benzene ring.

The compounds of formula (I) also may be transformed into suitable, pharmaceutically acceptable salts. The term pharmaceutically acceptable salt of a compound refers to a salt that is pharmaceutically acceptable, as defined herein, and that possesses the desired pharmacological activity of the parent compound. Pharmaceutically acceptable salts include acid addition salts formed with inorganic acids, e.g. hydrochloric acid, hydrobromic acid, sulphuric acid, nitric acid, phosphoric acid; or formed with organic acids, e.g. acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, etc.

In the preparation of acid addition salts, preferably such acid are used which form suitably therapeutically acceptable salts. Examples of such acids are hydrohalogen acids, sulfuric acid, phosphoric acid, nitric acid, aliphatic, alicyclic, aromatic or heterocyclic carboxylic or sulfonic acids, such as formic acid, acetic acid, propionic acid, succinic acid, glycolic acid, lactic acid, malic acid, tartaric acid, citric acid, ascorbic acid, maleic acid, hydroxymaleic acid, pyruvic acid, p-hydroxybenzoic acid, embonic acid, methanesulfonic acid, ethanesulfonic acid, hydroxyethanesulfonic acid, halogenbenzenesulfonic acid, toluenesulfonic acid or naphthalenesulfonic acid.

Whenever a chiral atom is present in a chemical structure, it is intended that all stereoisomers associated with that chiral atom are encompassed by the structure, unless otherwise specified. Using the Cahn-Ingold-Prelog RS notational system, any asymmetric atom may be present in the (R)- or (S)-configuration, and the compound may be present as a mixture of its stereoisomers, e.g. a racemic mixture, or one stereoisomer only, each being within the scope of the present invention.

In some embodiments, the compound of formula (I) is selected from the compounds represented in Table 1 herein below.

TABLE 1

| Name | Formula |
|---|---|
| 3-(3,4-dimethoxyphenyl)-2,6-dimethyl-N- (pyridin-4-ylmethyl)imidazo[1,2-b]pyridazin- 8-amine | 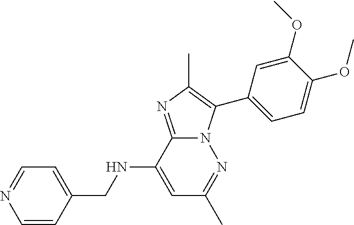 [SMILES: COc1ccc(-c2c(C)nc3c(NCc4ccncc4)cc(C)nn23)cc1OC] |
| 3-(3,4-dimethoxyphenyl)-2,6-dimethyl-N-((2- methylpyridin-4-yl)methyl)imidazo[1,2- b]pyridazin-8-amine | 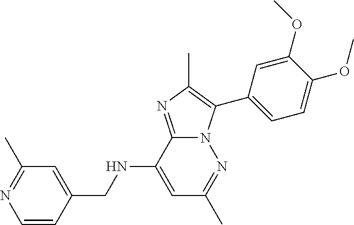 [SMILES: COc1ccc(-c2c(C)nc3c(NCc4ccnc(C)c4)cc(C)nn23)cc1OC] |
| 3-(3,4-dimethoxyphenyl)-N-((2- fluoropyridin-4-yl)methyl)-2,6- dimethylimidazo[1,2-b]pyridazin-8-amine | 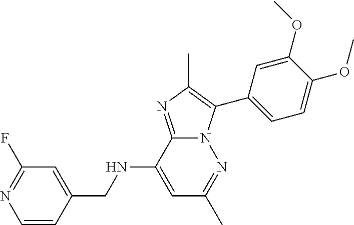 [SMILES: COc1ccc(-c2c(C)nc3c(NCc4ccnc(F)c4)cc(C)nn23)cc1OC] |
| 3-(1,3-dimethyl-1H-indazol-5-yl)-2,5- dimethyl-N-[(2-fluoropyridin-4- yl)methyl]pyrazolo[1,5-a]pyrimidin-7-amine | 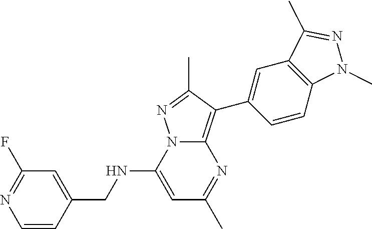 [SMILES: Cc1cc(NCc2ccnc(F)c2)n2nc(C)c(-c3ccc4c(c3)c(C)nn4C)c2n1] |
| 3-(1,3-dimethyl-1H-indazol-5-yl)-2,5- dimethyl-N-[(2-methylpyridin-4- yl)methyl]pyrazolo[1,5-a]pyrimidin-7-amine | 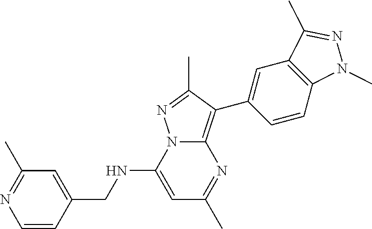 [SMILES: Cc1cc(CNc2cc(C)nc3c(-c4ccc5c(c4)c(C)nn5C)c(C)nn23)ccn1] |
| 3-(1,3-dimethyl-1H-indazol-5-yl)-2,5- dimethyl-N-[(2-ethylpyridin-4- yl)methyl]pyrazolo[1,5-a]pyrimidin-7-amine | 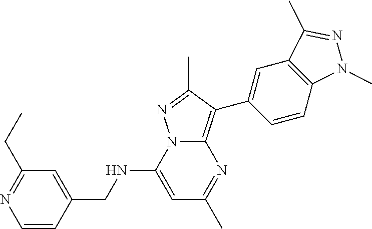 [SMILES: CCc1cc(CNc2cc(C)nc3c(-c4ccc5c(c4)c(C)nn5C)c(C)nn23)ccn1] |
| 3-(1,3-dimethyl-1H-indazol-5-yl)-2,5- dimethyl-N-[(2-methoxypyridin-4- yl)methyl]pyrazolo[1,5-a]pyrimidin-7-amine | 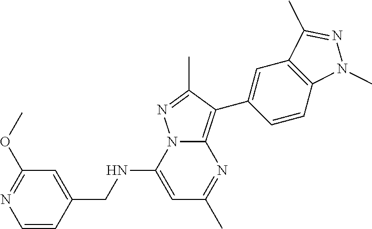 [SMILES: COc1cc(CNc2cc(C)nc3c(-c4ccc5c(c4)c(C)nn5C)c(C)nn23)ccn1] |
| 3-(1,3-dimethyl-1H-indazol-5-yl)-2,5- dimethyl-N-[(2-cyanopyridin-4- yl)methyl]pyrazolo[1,5-a]pyrimidin-7-amine | 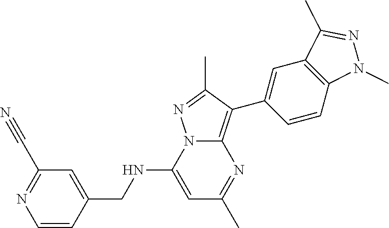 [SMILES: Cc1cc(NCc2ccnc(C#N)c2)n2nc(C)c(-c3ccc4c(c3)c(C)nn4C)c2n1] |
| 3-(1,3-dimethyl-1H-indazol-5-yl)-2,5- dimethyl-N-[(2-trifluoromethylpyridin-4- yl)methyl]pyrazolo[1,5-a]pyrimidin-7-amine | 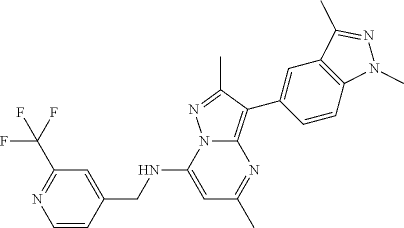 [SMILES: Cc1cc(NCc2ccnc(C(F)(F)F)c2)n2nc(C)c(-c3ccc4c(c3)c(C)nn4C)c2n1] |
| N-[(4-chlorophenyl)methyl]-3-(3,4- dimethoxyphenyl)-2,5-dimethyl-pyrazolo[1,5- a]pyrimidin-7-amine | 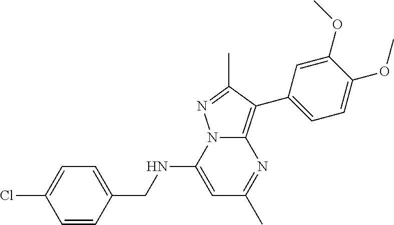 [SMILES: COc1ccc(-c2c(C)nn3c(NCc4ccc(Cl)cc4)cc(C)nc23)cc1OC] |
| 3-(3,4-dimethoxyphenyl)-N-[(4- methoxyphenyl)methyl]-2,5-dimethyl- pyrazolo[1,5-a]pyrimidin-7-amine | 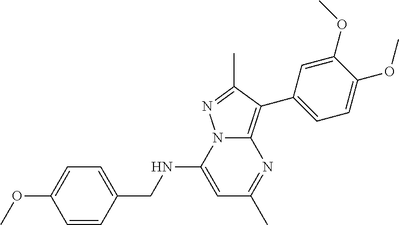 |
| 3-(3,4-dimethoxyphenyl)-2,5-dimethyl-N-(p- tolylmethyl)pyrazolo[1,5-a]pyrimidin-7- amine | 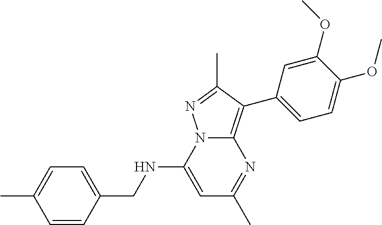 |
| N-(1,3-benzodioxol-5-ylmethyl)-3-(3,4- dimethoxyphenyl)-2,5-dimethyl-pyrazolo[1,5- a]pyrimidin-7-amine | 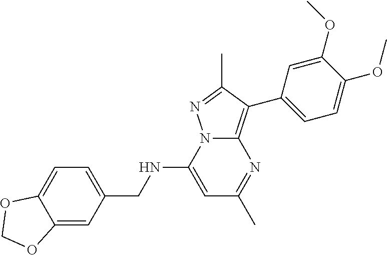 [SMILES: COc1ccc(-c2c(C)nn3c(NCc4ccc5c(c4)OCO5)cc(C)nc23)cc1OC] |
| 3-(3,4-dimethoxyphenyl)-N-[(4- fluorophenyl)methyl]-2,5-dimethyl- pyrazolo[1,5-a]pyrimidin-7-amine | 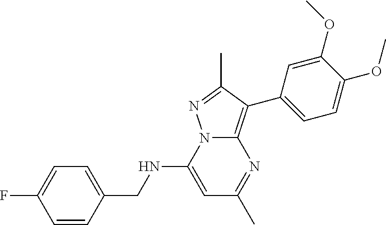 [SMILES: COc1ccc(-c2c(C)nn3c(NCc4ccc(F)cc4)cc(C)nc23)cc1OC] |
| 3-(3,4-dimethoxyphenyl)-2,5-dimethyl-N-(2- pyridylmethyl)pyrazolo[1,5-a]pyrimidin-7- amine | 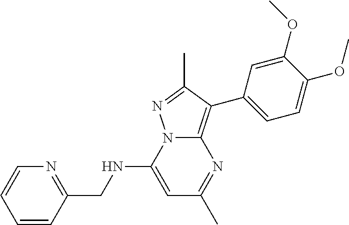 [SMILES: COc1ccc(-c2c(C)nn3c(NCc4ccccn4)cc(C)nc23)cc1OC] |
| N-benzyl-3-(3,4-dimethoxyphenyl)-2,5- dimethyl-pyrazolo[1,5-a]pyrimidin-7-amine | 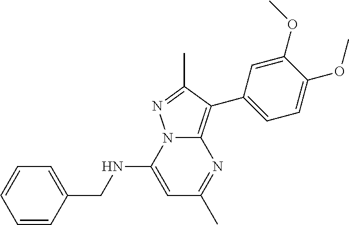 [SMILES: COc1ccc(-c2c(C)nn3c(NCc4ccccc4)cc(C)nc23)cc1OC] |
| 3-(3,4-dimethoxyphenyl)-2,5-dimethyl-N-[[4- (trifluoromethyl)phenyl]methyl]pyrazolo[1,5- a]pyrimidin-7-amine | 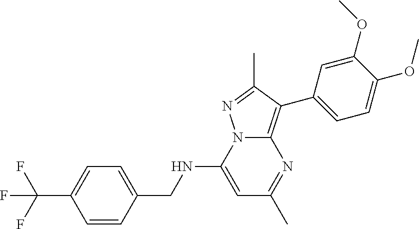 [SMILES: COc1ccc(-c2c(C)nn3c(NCc4ccc(C(F)(F)F)cc4)cc(C)nc23)cc1OC] |
| 3-(3,4-dimethoxyphenyl)-2,5-dimethyl-N-(4- pyridylmethyl)pyrazolo[1,5-a]pyrimidin-7- amine | 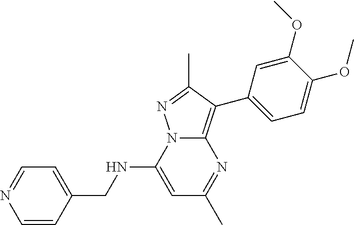 [SMILES: COc1ccc(-c2c(C)nn3c(NCc4ccncc4)cc(C)nc23)cc1OC] |
| 3-(3,4-dimethoxyphenyl)-2,5-dimethyl-N-[[4- (trifluoromethoxy)phenyl]methyl]pyrazolo[1, 5-a]pyrimidin-7-amine | 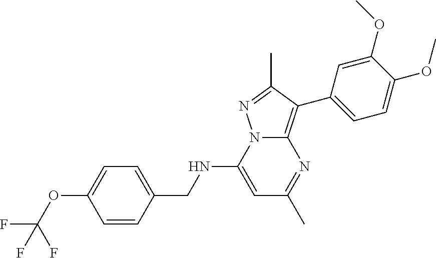 [SMILES: COc1ccc(-c2c(C)nn3c(NCc4ccc(OC(F)(F)F)cc4)cc(C)nc23)cc1OC] |
| N-[4-[[[3-(3,4-dimethoxyphenyl)-2,5- dimethyl-pyrazolo[1,5-a]pyrimidin-7- yl]amino methyl]phenyl]acetamide | 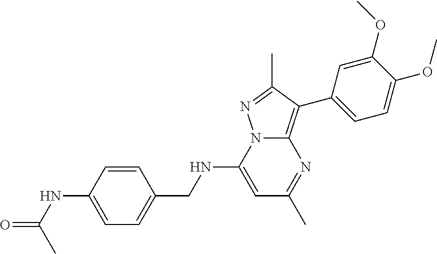 [SMILES: COc1ccc(-c2c(C)nn3c(NCc4ccc(NC(C)=O)cc4)cc(C)nc23)cc1OC] |
| 3-(3,4-dimethoxyphenyl)-N-[(4- dimethylaminophenyl)methyl]-2,5-dimethyl- pyrazolo[1,5-a]pyrimidin-7-amine | 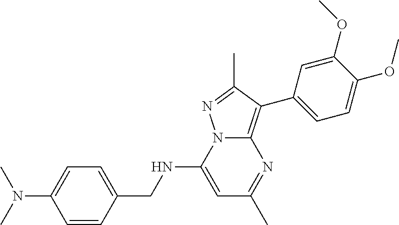 |
| 3-(3,4-dimethoxyphenyl)-N-[(6-methoxy-3- pyridyl)methyl]-2,5-dimethyl-pyrazolo[1,5- a]pyrimidin-7-amine | 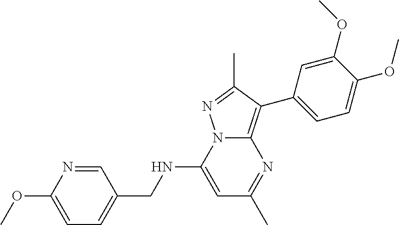 |
| N-[4-[[[3-(3,4-dimethoxyphenyl)-2,5- dimethyl-pyrazolo[1,5-a]pyrimidin-7- yl]amino]methyl]phenyl]methanesulfonamide | 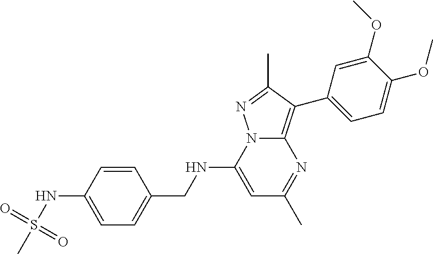 [SMILES: COc1ccc(-c2c(C)nn3c(NCc4ccc(NS(C)(=O)=O)cc4)cc(C)nc23)cc1OC] |
| 4-[[[3-(3,4-dimethoxyphenyl)-2,5-dimethyl- pyrazolo[1,5-a]pyrimidin-7- yl]amino]methyl]phenol | 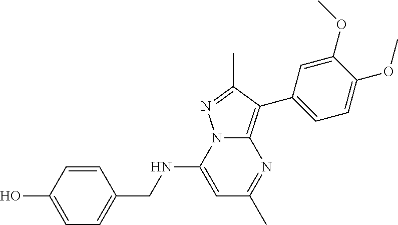 [SMILES: COc1ccc(-c2c(C)nn3c(NCc4ccc(O)cc4)cc(C)nc23)cc1OC] |
| 3-(3,4-dimethoxyphenyl)-2,5-dimethyl-N-[(3- methylsulfonylphenyl)methyl]pyrazolo[1,5- a]pyrimidin-7-amine | 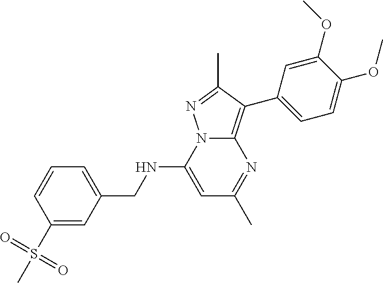 [SMILES: COc1ccc(-c2c(C)nn3c(NCc4cccc(S(C)(=O)=O)c4)cc(C)nc23)cc1OC] |
| 3-(3,4-dimethoxyphenyl)-2,5-dimethyl-N-[(4- methylsulfonylphenyl)methyl]pyrazolo[1,5- a]pyrimidin-7-amine | 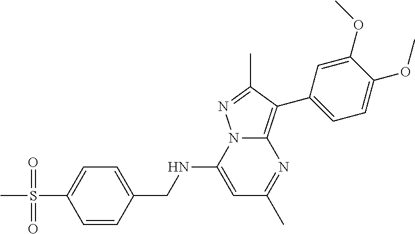 |
| N-[(4-tert-butylphenyl)methyl]-3-(3,4- dimethoxyphenyl)-2,5-dimethyl-pyrazolo[1,5- a]pyrimidin-7-amine | 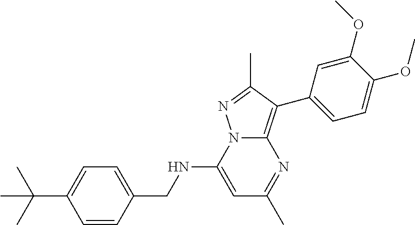 [SMILES: COc1ccc(-c2c(C)nn3c(NCc4ccc(C(C)(C)C)cc4)cc(C)nc23)cc1OC] |
| 3-(3,4-dimethoxyphenyl)-2,5-dimethyl-N-[(2- methylpyrimidin-4-yl)methyl]pyrazolo[1,5- a]pyrimidin-7-amine | 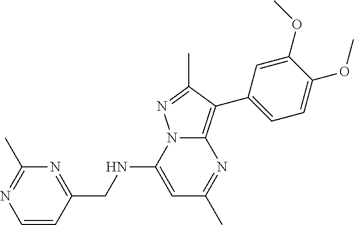 [SMILES: COc1ccc(-c2c(C)nn3c(NCc4ccnc(C)n4)cc(C)nc23)cc1OC] |
| 3-(3,4-dimethoxyphenyl)-2,5-dimethyl-N-[(6- methyl-3-pyridyl)methyl]pyrazolo[1,5- a]pyrimidin-7-amine | 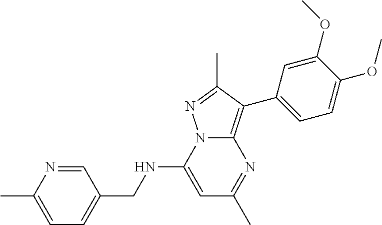 |
| 1-[4-[[3-(3,4-dimethoxyphenyl)-2,5- dimethyl-pyrazolo[1,5-a]pyrimidin-7- yl]amino methyl]phenyl]ethanone | 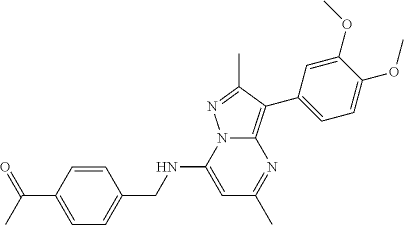 [SMILES: COc1ccc(-c2c(C)nn3c(NCc4ccc(C(C)=O)cc4)cc(C)nc23)cc1OC] |
| 3-(3,4-dimethoxyphenyl)-2,5-dimethyl-N-(1- naphthylmethyl)pyrazolo[1,5-a]pyrimidin-7- amine | 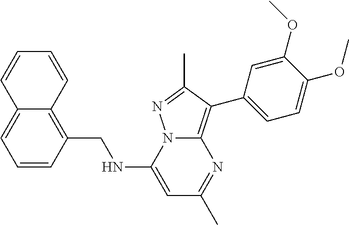 [SMILES: COc1ccc(-c2c(C)nn3c(NCc4cccc5ccccc45)cc(C)nc23)cc1OC] |
| 4-[[[3-(3,4-dimethoxyphenyl)-2,5-dimethyl- pyrazolo[1,5-a]pyrimidin-7- yl]amino]methyl]benzenesulfonamide | 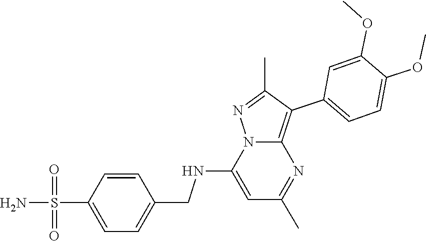 [SMILES: COc1ccc(-c2c(C)nn3c(NCc4ccc(S(N)(=O)=O)cc4)cc(C)nc23)cc1OC] |
| 8-(3,4-dimethoxyphenyl)-N-[(4- fluorophenyl)methyl]-2,7-dimethyl- pyrazolo[1,5-a][1,3,5]triazin-4-amine | 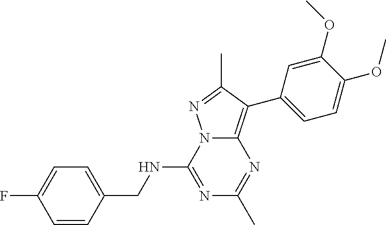 [SMILES: COc1ccc(-c2c(C)nn3c(NCc4ccc(F)cc4)nc(C)nc23)cc1OC] |
| 8-(3,4-dimethoxyphenyl)-2,7-dimethyl-N-[[4- (trifluoromethyl)phenyl]methyl]pyrazolo[1,5- a][1,3,5]triazin-4-amine | 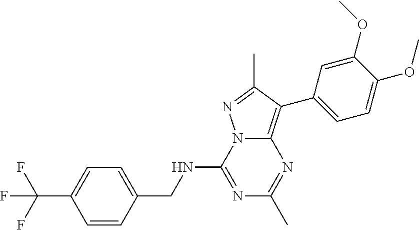 [SMILES: COc1ccc(-c2c(C)nn3c(NCc4ccc(C(F)(F)F)cc4)nc(C)nc23)cc1OC] |
| 8-(3,4-dimethoxyphenyl)-2,7-dimethyl-N-(p- tolylmethyl)pyrazolo[1,5-a][1,3,5]triazin-4- amine | 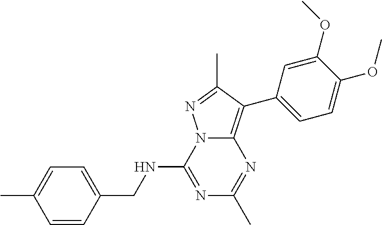 |
| 8-(3,4-dimethoxyphenyl)-N-[(4- isopropylphenyl)methyl]-2,7-dimethyl- pyrazolo[1,5-a][1,3,5]triazin-4-amine | 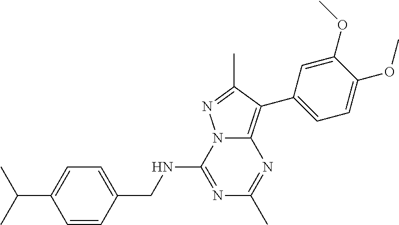 [SMILES: COc1ccc(-c2c(C)nn3c(NCc4ccc(C(C)C)cc4)nc(C)nc23)cc1OC] |
| 8-(3,4-dimethoxyphenyl)-2,7-dimethyl-N-(2- pyridylmethyl)pyrazolo[1,5-a][1,3,5]triazin-4- amine | 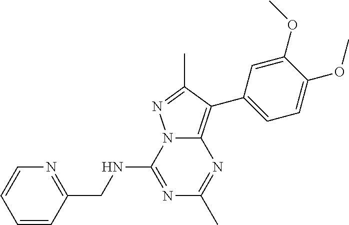 [SMILES: COc1ccc(-c2c(C)nn3c(NCc4ccccn4)nc(C)nc23)cc1OC] |
| 8-(3,4-dimethoxyphenyl)-2,7-dimethyl-N-(4- pyridylmethyl)pyrazolo[1,5-a][1,3,5]triazin-4- amine | 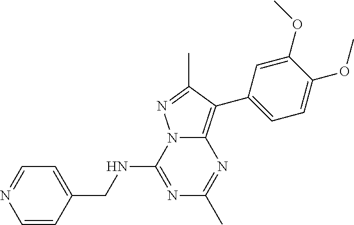 [SMILES: COc1ccc(-c2c(C)nn3c(NCc4ccncc4)nc(C)nc23)cc1OC] |
| N-[(4-chlorophenyl)methyl]-8-(3,4- dimethoxyphenyl)-2,7-dimethyl-pyrazolo[1,5- a][1,3,5]triazin-4-amine | 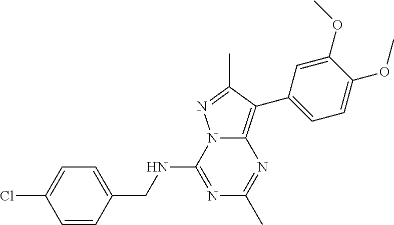 [SMILES: COc1ccc(-c2c(C)nn3c(NCc4ccc(Cl)cc4)nc(C)nc23)cc1OC] |
| N-[4-[[8-(3,4-dimethoxyphenyl)-2,7- dimethyl-pyrazolo[1,5-a][1,3,5]triazin-4- yl]amino methyl]phenyl]acetamide | 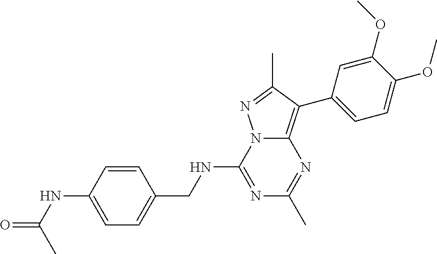 [SMILES: COc1ccc(-c2c(C)nn3c(NCc4ccc(NC(C)=O)cc4)nc(C)nc23)cc1OC] |
| 8-(3,4-dimethoxyphenyl)-N-[(4- methoxyphenyl)methyl]-2,7-dimethyl- pyrazolo[1,5-a][1,3,5]triazin-4-amine | 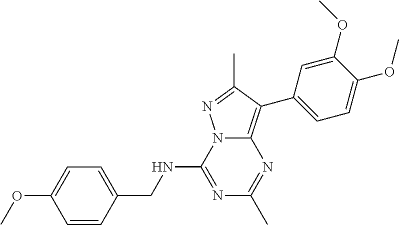 |
| 8-(3,4-dimethoxyphenyl)-2,7-dimethyl-N-[(3- methylsulfonylphenyl)methyl]pyrazolo[1,5- a][1,3,5]triazin-4-amine | 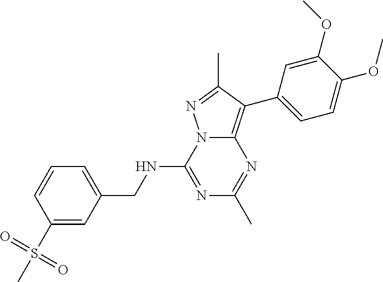 [SMILES: COc1ccc(-c2c(C)nn3c(NCc4cccc(S(C)(=O)=O)c4)nc(C)nc23)cc1OC] |
| 8-(3,4-dimethoxyphenyl)-2,7-dimethyl-N-[(1- oxidopyridin-1-ium-4-yl)methyl]pyrazolo[1,5- a][1,3,5]triazin-4-amine | 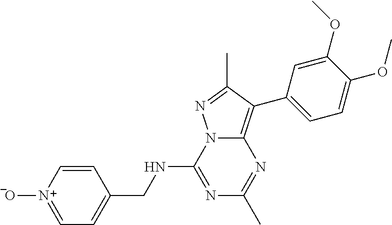 [SMILES: COc1ccc(-c2c(C)nn3c(NCc4cc[n+]([O-])cc4)nc(C)nc23)cc1OC] |
| 8-(3,4-dimethoxyphenyl)-N-[(6-methoxy-3- pyridyl)methyl]-2,7-dimethyl-pyrazolo[1,5- a][1,3,5]triazin-4-amine | 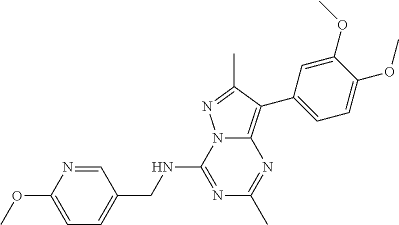 |
| 4-[[[8-(3,4-dimethoxyphenyl)-2,7-dimethyl- pyrazolo[1,5-a][1,3,5]triazin-4- yl]amino methyl]benzenesulfonamide | 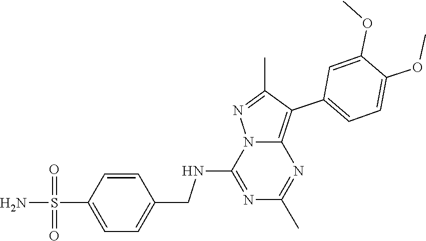 [SMILES: COc1ccc(-c2c(C)nn3c(NCc4ccc(S(N)(=O)=O)cc4)nc(C)nc23)cc1OC] |
| N-(1,3-benzodioxol-5-ylmethyl)-8-(3,4- dimethoxyphenyl)-2,7-dimethyl-pyrazolo[1,5- a][1,3,5]triazin-4-amine | 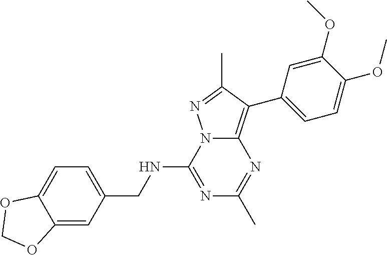 [SMILES: COc1ccc(-c2c(C)nn3c(NCc4ccc5c(c4)OCO5)nc(C)nc23)cc1OC] |
| 4-[[[8-(3,4-dimethoxyphenyl)-2,7-dimethyl- pyrazolo[1,5-a][1,3,5]triazin-4- yl]amino methyl]phenol | 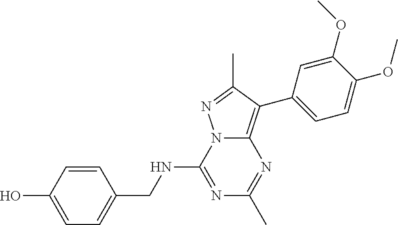 [SMILES: COc1ccc(-c2c(C)nn3c(NCc4ccc(O)cc4)nc(C)nc23)cc1OC] |
| N-[4-[[8-(3,4-dimethoxyphenyl)-2,7- dimethyl-pyrazolo[1,5-a][1,3,5]triazin-4- yl]amino]methyl]phenyl]methanesulfonamide | 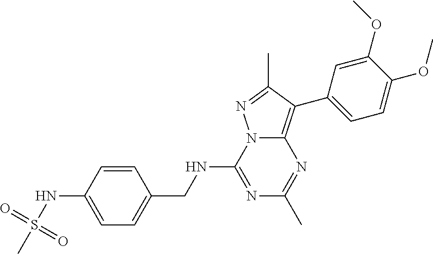 [SMILES: COc1ccc(-c2c(C)nn3c(NCc4ccc(NS(C)(=O)=O)cc4)nc(C)nc23)cc1OC] |
| N-benzyl-8-(3,4-dimethoxyphenyl)-2,7- dimethyl-pyrazolo[1,5-a][1,3,5]triazin-4- amine | 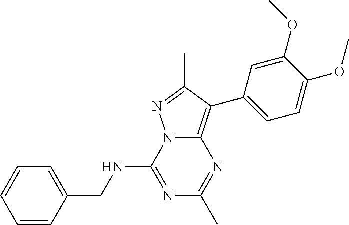 [SMILES: COc1ccc(-c2c(C)nn3c(NCc4ccccc4)nc(C)nc23)cc1OC] |
| 8-(3,4-dimethoxyphenyl)-2,7-dimethyl-N-(2- thienylmethyl)pyrazolo[1,5-a][1,3,5]triazin-4- amine | 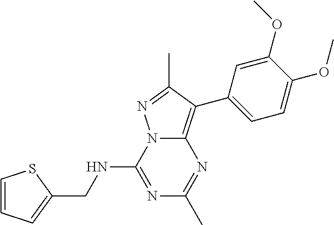 [SMILES: COc1ccc(-c2c(C)nn3c(NCc4cccs4)nc(C)nc23)cc1OC] |
| 8-(3,4-dimethoxyphenyl)-2,7-dimethyl-N-(3- pyridylmethyl)pyrazolo[1,5-a][1,3,5]triazin-4- amine | 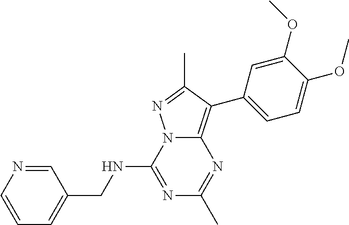 [SMILES: COc1ccc(-c2c(C)nn3c(NCc4cccnc4)nc(C)nc23)cc1OC] |
| 8-(3,4-dimethoxyphenyl)-2,7-dimethyl-N-[(5- methyl-2-furyl)methyl]pyrazolo[1,5- a][1,3,5]triazin-4-amine | 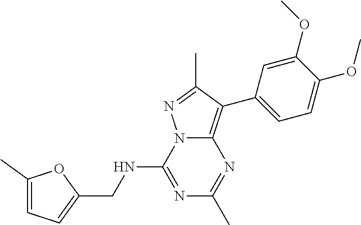 |
| methyl N-[4-[[[8-(3,4-dimethoxyphenyl)-2,7- dimethyl-pyrazolo[1,5-a][1,3,5]triazin-4- yl]amino]methyl]phenyl]sulfonylcarbamate | 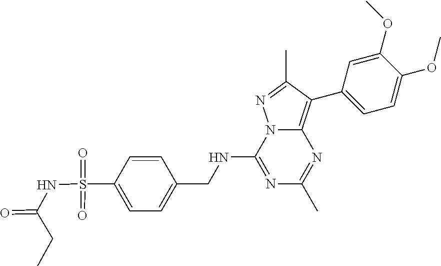 [SMILES: CCC(=O)NS(=O)(=O)c1ccc(CNc2nc(C)nc3c(-c4ccc(OC)c(OC)c4)c(C)nn23)cc1] |
| N-[4-[[[8-(3,4-dimethoxyphenyl)-2,7- dimethyl-pyrazolo[1,5-a][1,3,5]triazin-4- yl]amino]methyl]phenyl]sulfonylpropanamide | 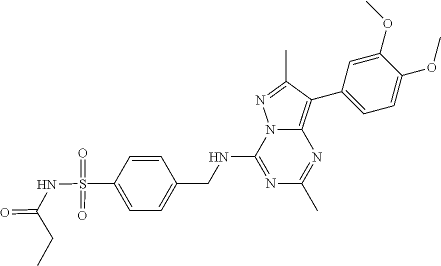 [SMILES: CCC(=O)NS(=O)(=O)c1ccc(CNc2nc(C)nc3c(-c4ccc(OC)c(OC)c4)c(C)nn23)cc1] |
| N-methyl-4-[[[8-(3,4-dimethoxyphenyl)-2,7- dimethyl-pyrazolo[1,5-a][1,3,5]triazin-4- yl]amino]methyl]benzenesulfonamide | 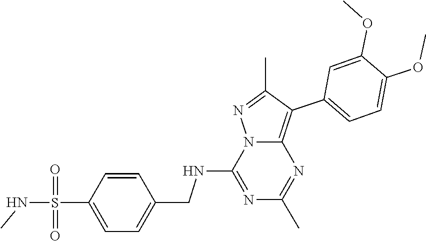 |
| 8-(3,4-dimethoxyphenyl)-2,7-dimethyl-N-[(1- methyl-1H-pyrazol-3-yl)methyl]pyrazolo[1,5- a][1,3,5]triazin-4-amine | 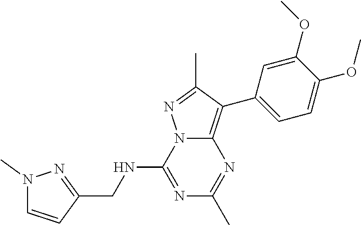 |
| 8-(3,4-dimethoxyphenyl)-2,7-dimethyl-N- [(1,3-oxazol-5-yl)methyl]pyrazolo[1,5- a][1,3,5]triazin-4-amine | 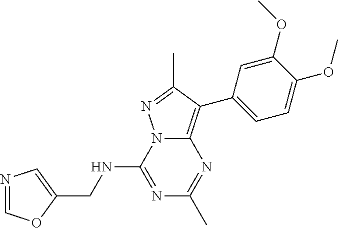 [SMILES: COc1ccc(-c2c(C)nn3c(NCc4cnco4)nc(C)nc23)cc1OC] |
| 8-(3,4-dimethoxyphenyl)-2,7-dimethyl-N-[(2- methylpyrimidin-5-yl)methyl]pyrazolo[1,5- a][1,3,5]triazin-4-amine | 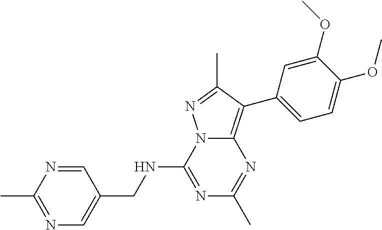 |
| 8-(3,4-dimethoxyphenyl)-2,7-dimethyl-N- [(pyridazin-4-yl)methyl]pyrazolo[1,5- a][1,3,5]triazin-4-amine | 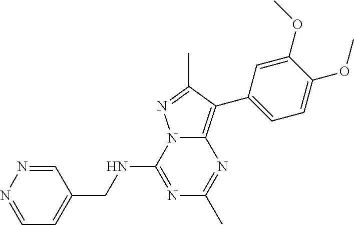 [SMILES: COc1ccc(-c2c(C)nn3c(NCc4ccnnc4)nc(C)nc23)cc1OC] |
| 8-(3,4-dimethoxyphenyl)-2,7-dimethyl-N-[(6- methylpyridin-3-yl)methyl]pyrazolo[1,5- a][1,3,5]triazin-4-amine | 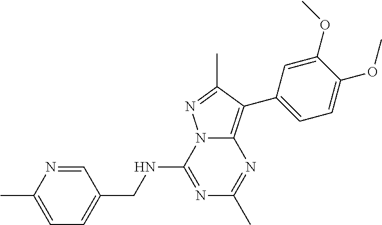 |
| 3-(1,3-dimethyl-1H-indazol-5-yl)-2,6- dimethyl-N-(pyridin-4-ylmethyl)imidazo[1,2- b]pyridazin-8-amine | 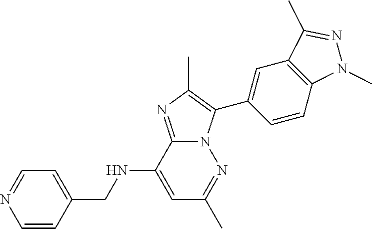 [SMILES: Cc1cc(NCc2ccncc2)c2nc(C)c(-c3ccc4c(c3)c(C)nn4C)n2n1] |
| 3-(1,3-dimethyl-1H-indazol-5-yl)-2,6- dimethyl-N-((6-methylpyridin-3- yl)methyl)imidazo[1,2-b]pyridazin-8-amine | 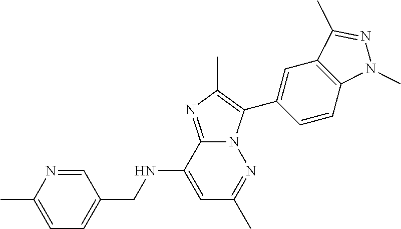 |
| N-(4-chlorobenzyl)-3-(1,3-dimethyl-1H- indazol-5-yl)-2,6-dimethylimidazo[1,2- b]pyridazin-8-amine | 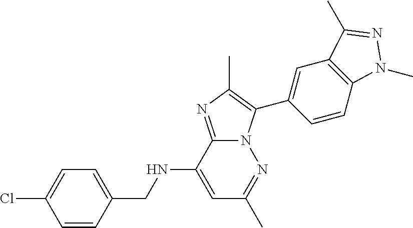 [SMILES: Cc1cc(NCc2ccc(Cl)cc2)c2nc(C)c(-c3ccc4c(c3)c(C)nn4C)n2n1] |
| 3-(1,3-dimethyl-1H-indazol-5-yl)-N-((6- methoxypyridin-3-yl)methyl)-2,6- dimethylimidazo[1,2-b]pyridazin-8-amine | 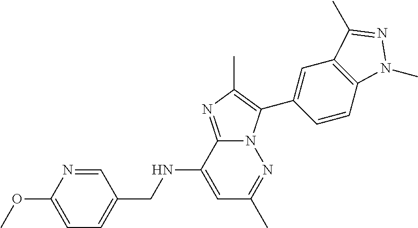 |
| 3-(1,3-dimethyl-1H-indazol-5-yl)-2,6- dimethyl-N-(thiophen-2- ylmethyl)imidazo[1,2-b]pyridazin-8-amine | 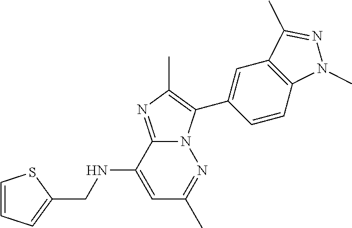 [SMILES: Cc1cc(NCc2cccs2)c2nc(C)c(-c3ccc4c(c3)c(C)nn4C)n2n1] |
| 3-(1,3-dimethyl-1H-indazol-5-yl)-N-(4- methoxybenzyl)-2,6-dimethylimidazo[1,2- b]pyridazin-8-amine | 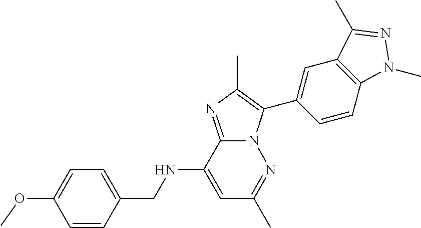 |
| 3-(1,3-dimethyl-1H-indazol-5-yl)-2,6- dimethyl-N-(4- (methylsulfonyl)benzyl)imidazo[1,2- b]pyridazin-8-amine | 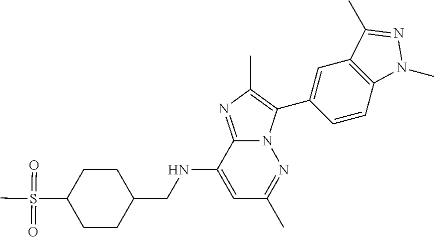 |
| 3-(1,3-dimethyl-1H-indazol-5-yl)-2,5- dimethyl-N-(pyridin-4- ylmethyl)pyrazolo[1,5-a]pyrimidin-7-amine | 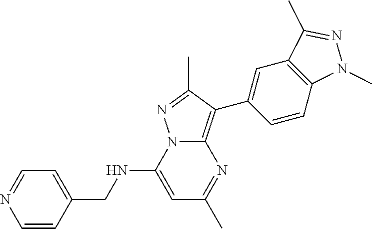 [SMILES: Cc1cc(NCc2ccncc2)n2nc(C)c(-c3ccc4c(c3)c(C)nn4C)c2n1] |
| 3-(1,3-dimethyl-1H-indazol-5-yl)-2,5- dimethyl-N-((6-methylpyridin-3- yl)methyl)pyrazolo[1,5-a]pyrimidin-7-amine | 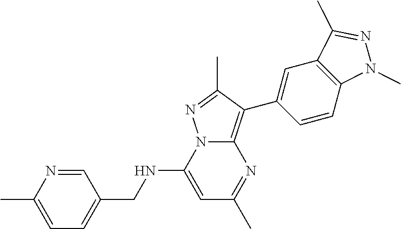 |
| 3-(1,3-dimethyl-1H-indazol-5-yl)-N-(4- methoxybenzyl)-2,5-dimethylpyrazolo[1,5- a]pyrimidin-7-amine | 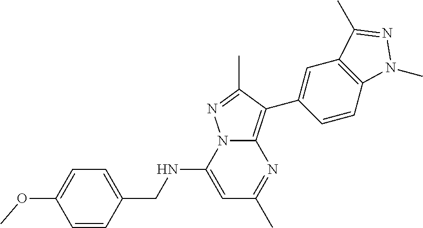 |
| N-(4-chlorobenzyl)-3-(1,3-dimethyl-1H- indazol-5-yl)-2,5-dimethylpyrazolo[1,5- a]pyrimidin-7-amine | 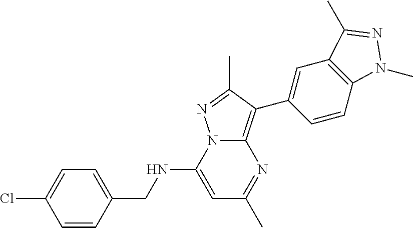 [SMILES: Cc1cc(NCc2ccc(Cl)cc2)n2nc(C)c(-c3ccc4c(c3)c(C)nn4C)c2n1] |
| 3-(1,3-dimethyl-1H-indazol-5-yl)-2,5- dimethyl-N-(4- (methylsulfonyl)benzyl)pyrazolo[1,5- a]pyrimidin-7-amine | 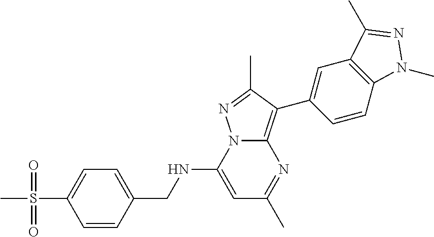 |
| 4-(((3-(1,3-dimethyl-1H-indazol-5-yl)-2,5- dimethylpyrazolo[1,5-a]pyrimidin-7- yl)amino)methyl)benzenesulfonamide | 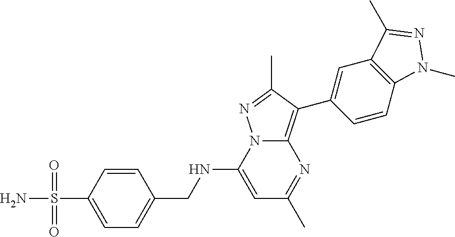 [SMILES: Cc1cc(NCc2ccc(S(N)(=O)=O)cc2)n2nc(C)c(-c3ccc4c(c3)c(C)nn4C)c2n1] |
| 3-(1,3-dimethyl-1H-indazol-5-yl)-N-((6- methoxypyridin-3-yl)methyl)-2,5- dimethylpyrazolo[1,5-a]pyrimidin-7-amine | 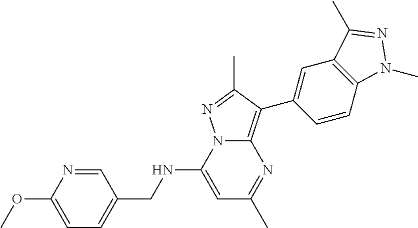 |
| 3-(1,3-dimethyl-1H-indazol-5-yl)-2,5- dimethyl-N-(thiophen-2- ylmethyl)pyrazolo[1,5-a]pyrimidin-7-amine | 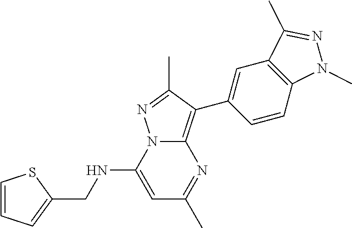 [SMILES: Cc1cc(NCc2cccs2)n2nc(C)c(-c3ccc4c(c3)c(C)nn4C)c2n1] |
| N-(4-(((3-(1,3-dimethyl-1H-indazol-5-yl)-2,5- dimethylpyrazolo[1,5-a]pyrimidin-7- yl)amino)methyl)phenyl)methanesulfonamide | 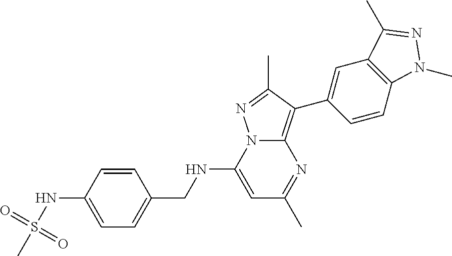 [SMILES: Cc1cc(NCc2ccc(NS(C)(=O)=O)cc2)n2nc(C)c(-c3ccc4c(c3)c(C)nn4C)c2n1] |
| 3-(1,3-dimethyl-1H-indazol-5-yl)-2,5- dimethyl-N-((6-trifluoromethylpyridin-3- yl)methyl)pyrazolo[1,5-a]pyrimidin-7-amine | 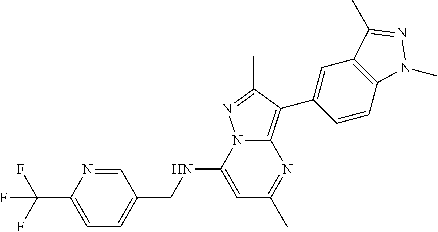 [SMILES: Cc1cc(NCc2ccc(C(F)(F)F)nc2)n2nc(C)c(-c3ccc4c(c3)c(C)nn4C)c2n1] |
| N-(benzyl)-3-(1,3-dimethyl-1H-indazol-5-yl)- 2,5-dimethylpyrazolo[1,5-a]pyrimidin-7- amine | 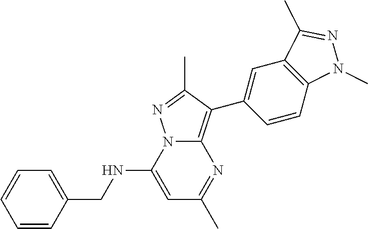 [SMILES: Cc1cc(NCc2ccccc2)n2nc(C)c(-c3ccc4c(c3)c(C)nn4C)c2n1] |
| N-(4-fluorobenzyl)-3-(1,3-dimethyl-1H- indazol-5-yl)-2,5-dimethylpyrazolo[1,5- a]pyrimidin-7-amine | 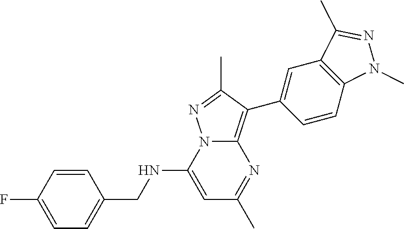 [SMILES: Cc1cc(NCc2ccc(F)cc2)n2nc(C)c(-c3ccc4c(c3)c(C)nn4C)c2n1] |
| 8-(1,3-dimethyl-1H-indazol-5-yl)-2,7- dimethyl-N-(pyridin-4- ylmethyl)pyrazolo[1,5-a][1,3,5]triazin-4- amine | 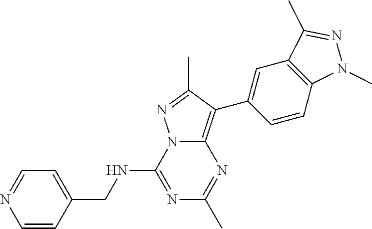 [SMILES: Cc1nc(NCc2ccncc2)n2nc(C)c(-c3ccc4c(c3)c(C)nn4C)c2n1] |
| 8-(1,3-dimethyl-1H-indazol-5-yl)-N-(4- methoxybenzyl)-2,7-dimethylpyrazolo[1,5- a][1,3,5]triazin-4-amine | 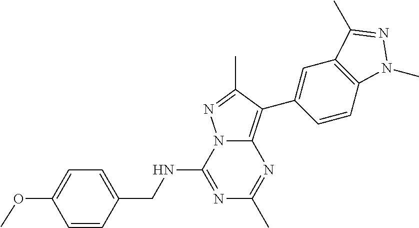 |
| 8-(1,3-dimethyl-1H-indazol-5-yl)-2,7- dimethyl-N-((6-methylpyridin-3- yl)methyl)pyrazolo[1,5-a][1,3,5]triazin-4- amine | 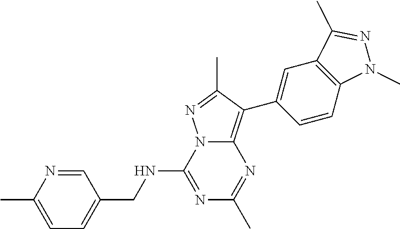 |
| 4-(((8-(1,3-dimethyl-1H-indazol-5-yl)-2,7- dimethylpyrazolo[1,5-a][1,3,5]triazin-4- yl)amino)methyl)benzenesulfonamide | 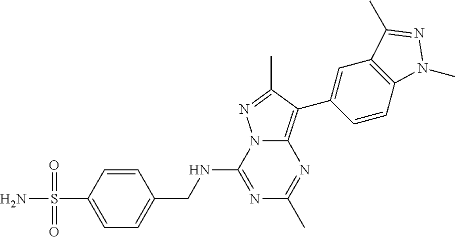 [SMILES: Cc1nc(NCc2ccc(S(N)(=O)=O)cc2)n2nc(C)c(-c3ccc4c(c3)c(C)nn4C)c2n1] |
| N-(4-chlorobenzyl)-8-(1,3-dimethyl-1H- indazol-5-yl)-2,7-dimethylpyrazolo[1,5- a][1,3,5]triazin-4-amine | 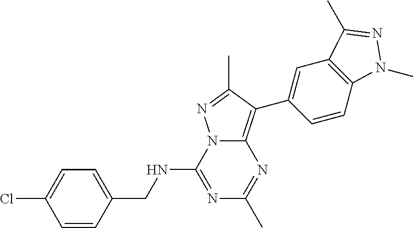 [SMILES: Cc1nc(NCc2ccc(Cl)cc2)n2nc(C)c(-c3ccc4c(c3)c(C)nn4C)c2n1] |
| 8-(1,3-dimethyl-1H-indazol-5-yl)-2,7- dimethyl-N-(4- (methylsulfonyl)benzyl)pyrazolo[1,5- a][1,3,5]triazin-4-amine | 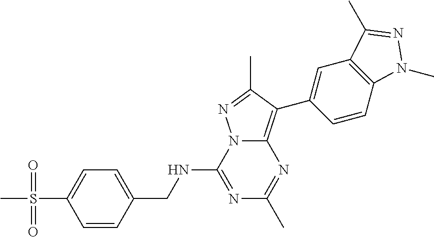 |
| N-(4-(((8-(1,3-dimethyl-1H-indazol-5-yl)-2,7- dimethylpyrazolo[1,5-a][1,3,5]triazin-4- yl)amino)methyl)phenyl)methanesulfonamide | 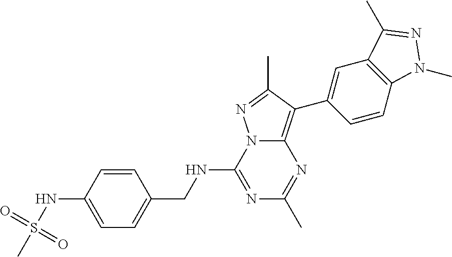 [SMILES: Cc1nc(NCc2ccc(NS(C)(=O)=O)cc2)n2nc(C)c(-c3ccc4c(c3)c(C)nn4C)c2n1] |
| 8-(1,3-dimethyl-1H-indazol-5-yl)-2,7- dimethyl-N-(thiophen-2- ylmethyl)pyrazolo[1,5-a][1,3,5]triazin-4- amine | 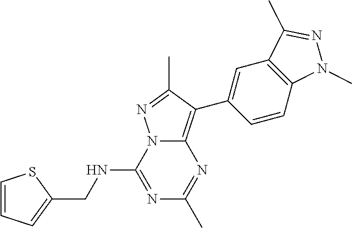 [SMILES: Cc1nc(NCc2cccs2)n2nc(C)c(-c3ccc4c(c3)c(C)nn4C)c2n1] |
| 8-(1,3-dimethyl-1H-indazol-5-yl)-N-((6- methoxypyridin-3-yl)methyl)-2,7- dimethylpyrazolo[1,5-a][1,3,5]triazin-4-amine | 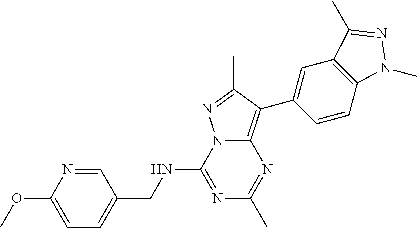 |

In some embodiments, the compound of formula (I) is 3-(1,3-dimethyl-1H-indazol-5-yl)-2,5-dimethyl-N-[(2-methylpyridin-4-yl)methyl]pyrazolo[1,5-a]pyrimidin-7-amine.

It is considered to be well within the knowledge of the person of ordinary skill in the art to synthesize and identify compounds of formula (I) as defined herein, and pharmaceutically acceptable salts thereof, by following methods described in literature, e.g. in PCT/EP2018/058522 (WO 2018/185120 A1), PCT/EP2015/051177 (WO 2015/110491 A2), PCT/EP2019/072220 (WO 2020/074159), and PCT/EP2020/072847 (WO 2021/032611) the contents of which publications are incorporated herein by reference.

### The Pharmaceutical Formulation

Further provided herein is a pharmaceutical formulation allowing for systemic delivery of a compound of formula (I) by transmucosal administration, preferably oral transmucosal administration. Thus, in a further aspect a pharmaceutical formulation is provided, comprising, as active ingredient, a compound of formula (I) or a pharmaceutically acceptable salt thereof, for transmucosal (preferably oral transmucosal) systemic administration of said compound or pharmaceutically acceptable salt thereof. In some embodiments, the oral formulation more particularly is a buccal formulation.

In some embodiments, the active ingredient is the compound of formula (I) in non-salt form. In some embodiments, the active ingredient is the compound of formula (I) in salt form, e.g. in the form of an acid addition salt.

In some embodiments, the formulation is a film as defined herein above, wherein the active ingredient is present in an amount of from 1 to 45% by weight, from 1 to 40% by weight, from 1 to 35% by weight, from 1 to 30% by weight, from 1 to 25% by weight, from 1 to 20% by weight, from 1 to 15% by weight, or from 1 to 10% by weight, relative to the total dry weight of the formulation.

In some embodiments, the active ingredient is present in an amount of from 1 to 50% by weight, preferably from 5 to 40% by weight, more preferably from 10 to 30% by weight, relative to the total dry weight of the formulation.

In some embodiments, the active ingredient is present in an amount of from 1 to 45% by weight, from 1 to 40% by weight, from 1 to 35% by weight, from 1 to 30% by weight, from 1 to 25% by weight, from 1 to 20% by weight, from 1 to 15% by weight, or from 1 to 10% by weight, relative to the total dry weight of the formulation.

In some embodiments, the active ingredient is present in an amount of from 5 to 45% by weight, from 5 to 40% by weight, from 5 to 35% by weight, from 5 to 30% by weight, from 5 to 25% by weight, from 5 to 20% by weight, from 5 to 15% by weight, or from 5 to 10% by weight, relative to the total dry weight of the formulation.

In some embodiments, the active ingredient is present in an amount of from 10 to 45% by weight, from 10 to 40% by weight, from 10 to 35% by weight, from 10 to 30% by weight, from 10 to 25% by weight, from 10 to 20% by weight, or from 10 to 15% by weight, relative to the total dry weight of the formulation.

In some embodiments, the active ingredient is present in an amount of from 15 to 45% by weight, from 15 to 40% by weight, from 15 to 35% by weight, from 15 to 30% by weight, from 15 to 25% by weight, or from 15 to 20% by weight, relative to the total dry weight of the formulation.

Preferably the film formulation is an oral film for transmucosal systemic administration, such as a buccal film or a sublingual film, and more preferably the film is mucoadhesive. In some embodiments, the film is a mucoadhesive buccal film for transmucosal systemic administration. In some embodiments, the film is a preferably mucoadhesive, immediate release film. In some embodiments, the film is a preferably mucoadhesive, immediate release oral film. In some embodiments, the film is a preferably mucoadhesive, immediate release buccal film.

Herein below, a film (or film formulation) for oral (preferably buccal) transmucosal administration may be referred to simply as an “oral film” (buccal film) or, in the alternative, as an “oral film formulation” (buccal film formulation). Preferably, the oral film is mucoadhesive.

The film formulation of the invention generally comprises a polymeric matrix formed from one or more, e.g. one or two, polymeric film forming agents, and a compound of formula (I) or a pharmaceutically acceptable salt thereof contained in said matrix. Optionally, the film may also include one or more excipients, such as one or more plasticizers, solubilizers, colorants, taste-masking agents, antioxidation agents, preservatives, penetration enhancers, and mucoadhesive agents.

If more than one polymeric film forming agent is present, at least one such agent is hydrophilic in nature. More preferably, all polymeric film forming agents are of hydrophilic nature.

The one or more film forming agents may be selected from, for example, polyvinylpyrrolidone (povidone), polyethylene oxide, polyvinyl alcohol, polyvinyl acetate, polyethylene glycol, polyacrylic acid, carboxyvinyl copolymers, copolymers of N-vinyl-2-pyrrolidone and vinyl acetate (copovidone), polyvinyl alcohol-polyethylene glycol copolymers, polydextrose, pullulan, hydroxypropylmethyl cellulose (hypromellose), hydroxyethyl cellulose, hydroxypropyl cellulose, carboxymethyl cellulose, methylcellulose, ethylcellulose, sodium alginate, xanthan gum, tragancath gum, carrageenan, guar gum, pectin, acacia gum, arabic gum, starch, gelatin, methacrylate polymers, maleic acid copolymers, methacrylic acid-ethyl acrylate copolymers, methacrylic acid and methacrylate based polymers, and methylmethacrylate copolymers.

In some embodiments, the film is formed from at least one cellulose derivative, e.g. selected from hydroxypropyl methylcellulose, hydroxypropyl cellulose, hydroxyethyl cellulose, and carboxymethylcellulose.

Commercial HPMCs may be identified by codes, for instance including a letter (E, F or K) that relates to the degree of substitution, where K grades have a methoxy substitution of 19-24% and a hydroxypropyl substitution of 7-12%, F grades have a methoxy substitution of 27-30% and a hydroxypropyl substitution of 4.0-7.5% and E grades have a methoxy substitution of 28-30% and a hydroxypropyl substitution of 7-12%.

In some embodiments, at least one film forming polymer is hydroxypropyl methylcellulose, for example of grade E, such as HPMC E3, HPMC E5, or HPMC E15, in particular HPMC E3, where the numbers 3, 5 and 15 (in E3, E5 and E15, respectively) denote the viscosity, in mPas, at 20° C. of a 2% aqueous solution of the HPMC.

In some embodiments, at least one film forming polymer is hydroxypropyl methylcellulose of grade K. In still further embodiments, at least one film forming polymer is hydroxypropyl methylcellulose of grade F.

In some embodiments, the polymer film matrix is formed from HPMC, e.g HPMC E3, HPMC E5, and/or HPMC E15, such as HPMC E3 and/or HPMC E5, in combination with at least one further film-forming polymer, such as a synthetic polymer, for example polyvinyl alcohol, povidone, or copovidone; e.g. copovidone.

In some embodiments, the polymer film matrix is formed from HPMC E3 in combination with at least one further film-forming polymer, such as a synthetic polymer, for example polyvinyl alcohol, povidone, or copovidone; e.g. copovidone.

In some embodiments, the film forming polymer comprises a graft copolymer of ethylene glycol and vinyl alcohol, such as Kollicoat© IR, which is an ethylene glycol and vinyl alcohol graft copolymer consisting of 75% polyvinyl alcohol units and 25% polyethylene glycol units.

In some embodiments, the film forming polymer comprises polyvinylpyrrolidone (CAS number 9003-39-8).

In some embodiments, the film forming polymer comprises copovidone and at least one further film forming agent, for example one or more cellulose derivatives, such as HPMC, e.g. HPMC of grade E, in particular HPMC E3.

Generally, the film formulation comprises film forming polymer in a total amount of from 25 to 99% by weight, relative to the total dry weight of the formulation. In some embodiments, the film formulation comprises film forming polymer(s) in a total amount of from 25 to 95% by weight, from 25 to 80% by weight, from 25 to 75% by weight, from 25 to 70% by weight, from 25 to 65% by weight, from 25 to 60% by weight, from 25 to 55% by weight, or from 25 to 50% by weight, relative to the total dry weight of the formulation.

In some embodiments, the film formulation comprises film forming polymer(s) in a total amount of from 30 to 99% by weight, from 30 to 95% by weight, from 30 to 80% by weight, from 30 to 75% by weight, from 30 to 70% by weight, from 30 to 65% by weight, from 30 to 60% by weight, from 30 to 55% by weight, or from 30 to 50% by weight, relative to the total dry weight of the formulation.

In some embodiments, the film formulation comprises film forming polymer(s) in a total amount of from 35 to 99% by weight, from 35 to 95% by weight, from 35 to 80% by weight, from 35 to 75% by weight, from 35 to 70% by weight, from 35 to 65% by weight, from 35 to 60% by weight, from 35 to 55% by weight, or from 35 to 50% by weight, relative to the total dry weight of the formulation.

In some embodiments, the film formulation comprises film forming polymer(s) in a total amount of from 40 to 99% by weight, 40 to 95% by weight, from 40 to 80% by weight, from 40 to 75% by weight, from 40 to 70% by weight, from 40 to 65% by weight, from 40 to 60% by weight, from 40 to 55% by weight, or from 40 to 50% by weight, relative to the total dry weight of the formulation.

In some embodiments, the film formulation comprises film forming polymer(s) in a total amount of from 45 to 99% by weight, 45 to 95% by weight, from 45 to 80% by weight, from 45 to 75% by weight, from 45 to 70% by weight, from 45 to 65% by weight, from 45 to 60% by weight, from 45 to 55% by weight, or from 45 to 50% by weight, relative to the total dry weight of the formulation.

In some embodiments, the film formulation comprises film forming polymer(s) in a total amount of from 50 to 99% by weight, 50 to 95% by weight, from 50 to 80% by weight, from 50 to 75% by weight, from 50 to 70% by weight, from 50 to 65% by weight, from 50 to 60% by weight, or from 50 to 55% by weight, relative to the total dry weight of the formulation.

In some particular embodiments, the film formulation comprises a first film forming polymer (F1), for example selected from copovidone and povidone, and a second, different, film forming polymer (F2), for example selected from cellulose derivatives, in a weight ratio of 1:20 to 20:1 of the first to the second film forming polymer. In some embodiments, the F1:F2 weight ratio is in the range of from 1:15 to 15:1, e.g. 1:10 to 10:1. In some of these embodiments, the F1:F2 weight ratio is at least 1:1, at least 2:1, at least 3:1, at least 4:1, at least 5:1, at least 6:1, at least 7:1 or at least 8:1. For example, in some embodiments, the F1:F2 weight ratio is in the range of from 1:1 to 15:1, from 2:1 to 15:1, from 3:1 to 15:1, from 4:1 to 15:1, from 5:1 to 15:1, from 6:1 to 15:1, from 7:1 to 15:1, or from 8:1 to 15:1. In some embodiments, the F1:F2 weight ratio is at most 14:1, at most 13:1, at most 12:1, at most 11:1, at most 10:1, or at most 9:1.

In some embodiments, the first film forming polymer (F1) is copovidone, and the second film forming polymer (F2) is HPMC, e.g. HPMC E, in particular HPMC E3.

In addition to the film forming agent, the film formulation of the invention optionally contains one or more further pharmaceutically acceptable excipients, for example selected from plasticizers, solubilizers, colorants, taste-masking agents, antioxidants, preservatives, surfactants, saliva-stimulating agents, buffering agents, and penetration enhancers.

In some embodiments, the film formulation comprises one or more plasticizers and optionally one or more further excipients, for example one or more solubilizers.

In some embodiments, the film formulation comprises one or more solubilizers and optionally one or more further excipients, for example one or more plasticizers.

Examples of plasticizers that can be used in the film formulation include glycerol monoacetate, diacetate or triacetate, triacetin, triethyl citrate, tributyl citrate, acetyl tributyl citrate, acetyl triethyl citrate, trioctyl citrate, acetyl trioctyl citrate, trihexyl citrate, dibutyl sebacate, polysorbate, cetyl alcohol, sodium diethylsulfosuccinate, polyethylene glycols, such as PEG 300 or PEG 400, polypropylene glycols, polyethylene-propylene glycols, and polyols, for example C3-C6 polyols, such as glycerol, sorbitol, xylitol, etc. In some embodiments, the plasticizer is selected from polyols, for example C3-C6 polyols, such as glycerol, sorbitol, and xylitol. In some embodiments, the plasticizer is glycerol.

In some embodiments, the film formulation comprises one or more plasticizers, in a total amount of from 0.5 to 25% by weight relative to the total dry weight of the formulation.

In some embodiments, the film formulation comprises plasticizer(s) in a total amount of from 0.5 to 20% by weight, from 0.5 to 15% by weight, from 0.5 to 10% by weight, from 0.5 to 8% by weight, from 0.5 to 7% by weight, from 0.5 to 6% by weight, from 0.5 to 5% by weight, or from 0.5 to 4% by weight, relative to the total dry weight of the formulation.

In some embodiments, the film formulation comprises plasticizer(s) in a total amount of from 1 to 25% by weight, from 1 to 20% by weight, from 1 to 15% by weight, from 1 to 10% by weight, from 1 to 8% by weight, from 1 to 7% by weight, from 1 to 6% by weight, from 1 to 5% by weight, or from 1 to 4% by weight, relative to the total dry weight of the formulation.

In some embodiments, the film formulation comprises plasticizer(s) in a total amount of from 2 to 25% by weight, from 2 to 20% by weight, from 2 to 15% by weight, from 2 to 10% by weight, from 2 to 8% by weight, from 2 to 7% by weight, from 2 to 6% by weight, from 2 to 5% by weight, or from 2 to 4% by weight, relative to the total dry weight of the formulation.

In some embodiments, the film formulation comprises plasticizer(s) in a total amount of from 3 to 25% by weight, from 3 to 20% by weight, from 3 to 15% by weight, from 3 to 10% by weight, from 3 to 8% by weight, from 3 to 7% by weight, from 3 to 6% by weight, from 3 to 5% by weight, or from 3 to 4% by weight, relative to the total dry weight of the formulation.

Examples of solubilizers that can be used in the film formulation include cyclodextrin and/or its derivatives, polyethylene glycol, polyvinylpyrrolidone, dextran, sugars such as sucrose, lactose or dextrose, polyols, such as mannitol, sorbitol or lactitol, sodium chloride, and mineral or organic acids.

In some embodiments, the solubilizing agent is an acid, such as phosphoric acid, citric acid, tartaric acid, malic acid, acetic acid, succinic acid, maleic acid, hydrochloric acid, benzoic acid, fumaric acid, or lactic acid. In some embodiments, the acid is hydrochloric acid or citric acid. For example, an acid may be added in an amount such as to provide a pH in the film forming formulation (generally an aqueous solution) of from about 2.5 to 4.3, from 2.6 to 4.2, from 2.7 to 4.1, preferably from 2.8 to 4.0.

In some embodiments, the film formulation additionally contains one or more further excipients, e.g. one or more further excipients selected from colorants, taste-masking agents, antioxidants, preservatives, surfactants, saliva stimulating agents, buffering agents, and penetration enhancers. Generally, any of these excipients, will be present in only minor amounts, for example, less than 5% by weight, less than 3% by weight, less than 2% by weight, less than 1% by weight, less than 0.5% by weight, or less than 0.1% by weight. In some embodiments, the total amount of all further excipients is less than 10% by weight, less than 5% by weight, less than 3% by weight, less than 2% by weight, less than 1% by weight, less than 0.5% by weight, or less than 0.1% by weight.

In some embodiments, no further excipient is present, viz. the film formulations includes only the active ingredient, the film forming polymer(s), and optionally one or more solubilizers and one or more plasticizers.

Suitable colorants may be selected from any of the colorants as conventionally used in the pharmaceutical industry, for example a pharmaceutical dye such as Tartrazine (E102), Allura Red (E129), Patent Blue (E131), or Brilliant Black BN (E151), or a pigment such as titanium dioxide.

Suitable taste masking agents may be selected from, for example, maltol, ethyl maltol, monosodium glutamate, glutamic acid, glutamates, purine-5-ribonucleotides, inosine, guanosine, adenosine 5-monophosphates, sugars, sweeteners, carboxylic acids (e.g., citric, malic, and tartaric), and xylitol.

Suitable antioxidants may be selected from, for example, propyl gallate, EDTA, copper(II) chloride, vitamin E and sodium metabisulfite.

Suitable preservatives may be selected from, for example, methyl propylparaben, propylparaben, benzalkonium chloride, propylene glycol, and benzoic acid.

Suitable surfactants may be selected from, for example, poloxamer 407, sodium lauryl sulfate, polysorbate, benzalkonium chloride, cetylpyridinium chloride, cetrimide and cetyltrimethylammonium bromide.

Suitable saliva stimulating agents may be selected from, for example, acids generally used in the food industry, such as ascorbic acid, malic acid, citric acid, tartaric acid, and lactic acid.

Suitable buffering agents may be selected from, for example, glycylglycine, malate, and formate buffers.

Suitable penetration enhancers may be selected from, for example, cationic surfactants, as mentioned herein above, such as benzalkonium chloride, and cetylpyridinium chloride.

A unit portion of the film formulation of the invention will generally be an oral, preferably mucoadhesive, patch or strip. Such a unit portion may have a surface area in the range of from 1 cm² to 8 cm², e.g. from 2 to 8 cm², from 3 to 8 cm², or from 4 to 8 cm², e.g. from 1 to 6 cm², from 2 to 6 cm², from 3 to 6 cm², or from 4 to 6 cm²; or from 1 to 4 cm², from 2 to 4 cm², or from 3 to 4 cm²; or from 1 to 3 cm², or from 2 to 3 cm².

The film will generally have a mean thickness in the range of from 0.1 mm to 0.5 mm, e.g. from 0.1 to 0.4 mm, from 0.1 to 0.35 mm, from 0.1 to 0.3 mm, from 0.1 to 0.25 mm, or from 0.1 to 0.2 mm; such as from 0.15 to 0.5 mm, from 0.15 to 0.4 mm, from 0.15 to 0.35 mm, from 0.15 to 0.3 mm, from 0.15 to 0.25 mm, from 0.15 to 0.2 mm; or from 0.2 to 0.5 mm, from 0.2 to 0.4 mm, from 0.2 to 0.35 mm, from 0.2 to 0.3 mm, or from 0.2 to 0.25 mm; or from 0.25 to 0.5 mm, from 0.25 to 0.4 mm, from 0.25 to 0.35 mm, or from 0.25 to 0.3 mm.

A suitable unit dose of a compound of formula (I), viz. the amount of active ingredient in a unit portion of a film formulation of the invention, may be in the range of, for example, 0.5 mg to 25 mg, or from 1 mg to 25 mg, or from 2 mg to 25 mg, or from 5 mg to 25 mg, or from 10 mg to 25 mg. In some embodiments, the unit dose is within the range of 0.5 mg to 20 mg, or from 1 mg to 20 mg, or from 2 mg to 20 mg, or from 5 mg to 20 mg, or from 10 mg to 20 mg. In some embodiments, the unit dose is within the range of 0.5 mg to 15 mg, or from 1 mg to 15 mg, or from 2 mg to 15 mg, or from 5 mg to 15 mg, or from 10 mg to 15 mg.

Such a unit dose may, for example be contained in a unit portion having a surface area of from 1 cm² to 8 cm², and a thickness in the range of from 0.1 mm to 0.5 mm.

In some embodiments, a bilayer or multilayer film includes a mucoadhesive layer containing the active ingredient which is placed against the oral mucosa and a second layer directed outwards from the mucosa serving as a protective barrier against abrasion from the tongue or mastication or simply against constant washing of the saliva. The outer layer also serves to favor the directed absorption of the active ingredient through the oral mucosa, and to reduce the enteric uptake in the gastrointestinal tract. The outer layer may be dissolvable or non-dissolvable. For example, a dissolvable backing layer for use with a transmucosal delivery device is described in international application No. PCT/US2004/014555 (WO 2004/103341), the content of which is incorporated herein by reference.

### Process for Preparing the Film Formulation

It is contemplated that the film formulation disclosed herein may be prepared by any of the known methods for preparing mucoadhesive films, e.g. by solvent casting or hot melt extrusion methods, or variants of these methods, semisolid casting and solid-dispersion extrusion methods. Preferably, the inventive film formulation is prepared by a solvent-casting method, i.e. a method that essentially consists of preparing an aqueous or hydro-alcoholic mixture of excipients and drug substance(s), casting the mixture onto a surface, allowing the mixture to dry on the surface and cutting the dried film into pieces of desirable form and size.

A dried film as provided herein generally will have a water content of less than about 10% by weight (i.e. % w/w), for example, a dry film as provided herein may have a water content of from 7% by weight to 10% by weight, or from 7% by weight to 9.5% by weight, or from 7% by weight to 9% by weight, e.g. from 8% by weight to 10% by weight, or from 8% by weight to 9.5% by weight, or from 8% by weight to 9% by weight.

The dry unit portions of the film may be packaged into suitable containers, e.g. resealable containers or disposable pouches of a water and air tight material suitable for use in packaging of products for human ingestion, e.g. a metallized polyethylene film (Alu/PET), or any other suitable laminate pouch, e.g. a PET/Alu/LDPE laminate pouch.

### The Use of the Film Formulation

The film of the invention may be administered to the mucosal surface of treated subject preferably as an oral film, e.g. as a buccal film or as a sublingual film, preferably as a mucoadhesive film, to provide for transmucosal delivery of the active ingredient to the systemic circulation of the treated subject. It is contemplated that treatment may be effected on a daily basis, once or several times a day, or on a weekly basis, or even more seldom, e.g. only when needed, to alleviate one or more symptoms of the treated disorder.

Suitable daily dosages typically are in the range of 1-500 mg, 1-200 mg, 1-100 mg, 1-50 mg, 1-20 mg, 1-10 mg, or 1-5 mg, depending upon numerous factors such as the severity of the disease to be treated, the age and relative health of the patient, the potency of the compound used, and the indication towards which the administration is directed, etc. One of ordinary skill in the medical field will be able, without undue experimentation and in reliance upon personal knowledge and the disclosure of this application, to ascertain a therapeutically effective amount of the compounds of the present invention for a given disease.

### The Virus Infection

A first aspect relates to a compound of formula (I) or a pharmaceutically acceptable salt thereof for use in the systemic treatment of a virus infection, by transmucosal administration of said compound or pharmaceutically acceptable salt thereof. Preferably, the transmucosal administration is oral transmucosal administration, e.g. buccal transmucosal administration. Generally, it is contemplated that the virus infection is one for which PI4K inhibition has a beneficial effect, more particularly PI4 KB inhibition, and it is contemplated that the virus is one that uses PI4K, in particular PI4 KB of the infected host.

In some embodiments, the virus is an RNA virus. In some embodiments, the RNA virus is a single-stranded RNA virus. In some embodiments, the virus is a non-enveloped single-stranded (+) RNA virus. In some embodiments, the RNA virus is an enveloped single-stranded (−) RNA virus.

In some embodiments, the virus is selected from viruses belonging to the Picornaviridae, Flaviviridae, Retroviridae, Filoviridae, Togaviridae, Papovaviridae, Papillomaviridae, Polyomaviridae, Caliciviridae, Coronaviridae, Hepeviridae, Bunyaviridae, Poxviridae or Orthomyxoviridae families, for example, the Picornaviridae, Flaviviridae, Caliciviridae, Filoviridae, Hepeviridae or Coronaviridae families, in particular viruses belonging to the Picornaviridae family (which may be referred to herein as picornaviruses) or the Pneumoviridae family (which may be referred to herein as pneumoviruses).

In some embodiments, the virus is selected from viruses belonging to the Picornaviridae family, such as viruses belonging to the Enterovirus genus; and viruses belonging to the Pneumoviridae family, such as viruses belonging to the Orthopneumovirus genus.

Some embodiments relate to a compound of formula (I), or a pharmaceutically acceptable salt thereof, for use in the systemic treatment of a disease or disorder linked to (or caused by) a Picornaviridae virus infection by transmucosal administration of said compound.

The picornavirus e.g. may an enterovirus (e.g. Entero, Coxsackie, Polio, Rhino) or a hepatovirus. For veterinary use, the picornavirus may be e.g. an Aphthovirus or a Teschovirus.

In some embodiments, the virus is an enterovirus, i.e. a virus belonging to the Enterovirus genus, for example, EV71.

Diseases that are considered to be linked to, caused by, or otherwise associated with virus infection, e.g. by picornaviruses, are e.g. neurodegenerative diseases such as multiple sclerosis, Parkinson's disease, amyotrophic lateral sclerosis, Alzheimer's disease, and Huntington's disease, poliomyelitis, encephalitis, meningitis, sepsis, cancer, paralysis, myocarditis, diabetes, common cold, hand-foot-and-mouth disease, herpangina, pleurodynia, diarrhea, mucocutaneous lesions, respiratory illness, conjunctivitis, myositis, and chronic fatigue syndrome. In some embodiments, the disease is selected from poliomyelitis, encephalitis, meningitis, sepsis, cancer, paralysis, myocarditis, diabetes, common cold, hand-foot-and-mouth disease, herpangina, pleurodynia, diarrhea, mucocutaneous lesions, respiratory illness, conjunctivitis, and myositis. In some embodiments, the disease is selected from poliomyelitis, encephalitis, meningitis, sepsis, cancer, paralysis, myocarditis, diabetes, common cold, and hand-foot-and-mouth disease.

In some embodiments, the disease is selected from poliomyelitis, myopericarditis, hemorrhagic conjunctivitis, pneumonia, herpangina, hand, foot, and mouth disease, acute flaccid paralysis and other polio-like illnesses, upper respiratory tract infections, acute exacerbations of chronic airway diseases (including chronic obstructive pulmonary disease, cystic fibrosis, asthma), and aseptic meningitis.

In some further embodiments, the virus infection is an infection by a virus belonging to the Pneumoviridae family. In some embodiments, the infection is an orthopneumoviral infection. In some of these embodiments, the virus is human orthopneumovirus (sometimes also referred to herein as human respiratory syncytial virus, or HRSV).

In some other embodiments, the infection is a metapneumoviral infection. In some embodiments, the virus is human metapneumovirus (HMPV), i.e. the infection is human metapneumoviral infection.

In some of the above embodiments, the infection is a respiratory tract infection, e.g. a lower tract respiratory infection, which may be a serious disease in particular in infants and elderly people, as well as in patients suffering from reduced respiratory function, such as patent suffering from asthma or chronic obstructive pulmonary disease. In some embodiments, therefore, a compound of the present invention is for use in the treatment of Pneumoviridae virus infection in a patient in need of such treatment. In some embodiments, the patient is an infant, an elderly person, or a patient suffering from reduced respiratory function.

Therefore, some embodiments relate to a compound of formula (I), or a pharmaceutically acceptable salt thereof, for use in the systemic treatment of a disease or disorder linked to (or caused by) a Pneumoviridae virus infection by transmucosal administration of said compound.

In some embodiments, the disease is an infection of the upper respiratory tract. In some embodiments the disease is an infection of the lower respiratory tract. In some embodiments, the disease is bronchiolitis. In some embodiments, the disease is pneumonia.

The Disorder for which PI4K Inhibition is Beneficial

A further aspect relates to a compound of formula (I) or a pharmaceutically acceptable salt thereof for use in the systemic treatment of a disorder for which PI4K inhibition is beneficial, by transmucosal administration of said compound or pharmaceutically acceptable salt thereof.

In some embodiments, the disorder is one wherein inhibition of PI4KA or PI4 KB is beneficial, e.g. a disorder wherein PI4 KB inhibition is beneficial.

A disorder for which PI4K inhibition is beneficial may be an infectious disease as well as a disease not having any known infectious cause, such as is the case in many cancers, autoimmune disorders, and inflammatory disorders.

Thus, in some embodiments, the disorder treated according to the invention is a disorder selected from infectious diseases, cancers, autoimmune disorders, and inflammatory disorders. In some of these embodiments, the disorder is an infectious disease. In some other embodiments, the disorder is cancer. In some further embodiments, the disorder is an autoimmune disorder. In still some further embodiments, the disorder is an inflammatory disorder.

In some further embodiments, a disorder for which PI4K inhibition is beneficial is caused by an infectious agent, such as a virus or a protozoon, e.g. selected from any of the viruses and protozoa as mentioned herein above. For example, the infectious agent may be a protozoon belonging to the Plasmodiidae family, such as Plasmodium falciparium, or belonging to the Cryptosporidiidae family, such as Cryptosporidium parvum, or Cryptosporidium hominis.

In some further embodiments, the disorder is selected from cancers, autoimmune disorders, and inflammatory disorders. In some further embodiments, the disorder is selected from pancreatitis, poliomyelitis, encephalitis, meningitis, sepsis, cancer, paralysis, a cardiac disease, diabetes, common cold, hand-foot-and-mouth disease, herpangina, pleurodynia, diarrhea, a mucocutaneous lesion, respiratory illness, conjunctivitis, myositis, chronic fatigue syndrome, a neuropsychiatric disease, a neurodegenerative disease or an inflammatory condition.

In some embodiments, the disorder is pancreatitis. In some embodiments, the disorder is poliomyelitis. In some embodiments, the disorder is encephalitis. In some embodiments, the disorder is meningitis. In some embodiments, the disorder is sepsis. In some embodiments, the disorder is cancer. In some embodiments, the disorder is paralysis. In some embodiments, the disorder is a cardiac disease. In some embodiments, the disorder is diabetes, e.g. diabetes type 1. In some embodiments, the disorder is common cold. In some embodiments, the disorder is hand-foot-and-mouth disease. In some embodiments, the disorder is herpangina. In some embodiments, the disorder is pleurodynia. In some embodiments, the disorder is diarrhea. In some embodiments, the disorder is a mucocutaneous lesion. In some embodiments, the disorder is respiratory illness. In some embodiments, the disorder is conjunctivitis. In some embodiments, the disorder is myositis. In some embodiments, the disorder is chronic fatigue syndrome. In some embodiments, the disorder is a neuropsychiatric disease. In some embodiments, the disorder is a neurodegenerative disease. In some embodiments, the disorder is an inflammatory condition.

### EXAMPLES

The film formulation, its preparation and use are illustrated in the following non-limiting examples.

### Example 1. Mucoadhesive Film Formulation

The ingredients listed in Table 2 were used in the indicated amounts to prepare a film formulation as described herein below.

TABLE 2

| | | Quantity |
|---|---|---|
| Ingredient | Function | (% w/w) |
| 3-(1,3-dimethyl-1H-indazol-5-yl)-2,5- | active ingredient | 10.0 |
| dimethyl-N-[(2-methylpyridin-4- | | |
| yl)methyl]pyrazolo[1,5-a]pyrimidin-7- | | |
| amine | | |
| Kollicoat ® IR | film forming agent | 15.3 |
| glycerol | plasticizer | 5.4 |
| water | solvent | 38.5 |
| 1M HCl (aq.) | solubilizing agent | 30.8 |

All ingredients except for the HCl (aq.) were admixed in a small vessel and the mixture was stirred using a magnetic stirrer. The HCl (aq.) was then added slowly until a slightly viscous, dark brown solution was obtained, having a pH of approx. 4.4. The solution was coated at a wet thickness of 1.0 mm onto a glass plate and dried at 40° C. for 60 minutes to give a brown film. The film was air dried at room temperature over the night. A fine, flexible film was obtained having a surface weight of 32 mg/cm², which corresponds to 10.5 mg of the active ingredient/cm².

### Example 2. Mucoadhesive Film Formulation

The ingredients listed in Table 3 were used in the indicated amounts to prepare a film formulation as described herein below.

TABLE 3

| | | Quantity |
|---|---|---|
| Ingredient | Function | (% w/w) |
| 3-(1,3-dimethyl-1H-indazol-5-yl)-2,5- | active ingredient | 13.0 |
| dimethyl-N-[(2-methylpyridin-4- | | |
| yl)methyl]pyrazolo[1,5-a]pyrimidin-7- | | |
| amine | | |
| HPMC Pharmacoat ® 603 | film forming agent | 3.8 |
| HPMC Metolose ® 60SH | film forming agent | 4.8 |
| glycerol | plasticizer | 7.0 |
| 80% ethanol (aq.) | solvent | 57.8 |
| absolute ethanol | solvent | 13.6 |

All ingredients except for the absolute ethanol were admixed in a small vessel and the mixture was stirred using a magnetic stirrer. After the addition of the absolute ethanol, a viscous, dark brown solution was obtained. The solution was coated at a wet thickness of 1.0 mm onto a glass plate and dried at 40° C. for 30 minutes to give a brown film with some holes. The film was air dried at room temperature over the night. An easily detachable film was obtained having a surface weight of 20 mg/cm², which corresponds to 9 mg of the active ingredient/cm².

### Example 3. Mucoadhesive Film Formulation

The ingredients listed in Table 4 were used in the indicated amounts to prepare a film formulation as described herein below.

TABLE 4

| | | Quantity |
|---|---|---|
| Ingredient | Function | (% w/w) |
| 3-(1,3-dimethyl-1H-indazol-5-yl)-2,5- | active ingredient | 8.5 |
| dimethyl-N-[(2-methylpyridin-4- | | |
| yl)methyl]pyrazolo[1,5-a]pyrimidin-7- | | |
| amine | | |
| Povidone | film forming agent | 14.9 |
| glycerol | plasticizer | 5.3 |
| 80% ethanol (aq.) | solvent | 71.3 |

All ingredients were admixed in a small vessel and the mixture was stirred using a magnetic stirrer until a viscous, dark brown solution was obtained. The solution was coated at a wet thickness of 1.0 mm onto a glass plate and dried at 40° C. for 30 minutes to give a brown film. The film was air dried at room temperature over the night. A film was obtained that was even and smooth, but somewhat difficult to remove from the

### Example 4. Mucoadhesive Film Formulation

Mucoadhesive films were prepared by solvent casting method, using HPMC as a mucoadhesive polymer and glycerol as a plasticizer. HPMC was dissolved in ultrapure water with continuous stirring using a magnetic stirrer to produce a 10% (w/v) solution. To the resulting solution, glycerol (15% of dry polymer weight) followed by 20 μL of Tween® 80 were added. The resulting solution were kept overnight for deaeration and swelling of HPMC. An amount of 100 mg of 3-(1,3-dimethyl-1H-indazol-5-yl)-2,5-dimethyl-N-[(2-methylpyridin-4-yl)methyl]pyrazolo[1,5-a]pyrimidin-7-amine as active ingredient was weighed and dissolved in 1N HCl. Once the active ingredient was completely dissolved in 1N HCl, volume was made up to 1 mL using polymer solution to get formulation with 100 mg/mL of final strength. The 200 μL (100 mg/mL) of polymer solution was poured into a multi-well glass mould having wells of 15 mm diameter. The films were dried at room temperature and then packed in aluminum foil and stored in an airtight container to maintain their integrity and elasticity.

### Example 5. Mucoadhesive Film Formulation

The ingredients listed in Table 5 were used in the indicated amounts to prepare a mucoadhesive film formulation as described herein below.

TABLE 5

| | | Quantity |
|---|---|---|
| Ingredient | Function | (g) |
| 3-(1,3-dimethyl-1H-indazol-5-yl)-2,5- | active ingredient | 40.0 |
| dimethyl-N-[(2-methylpyridin-4- | | |
| yl)methyl]pyrazolo[1,5-a]pyrimidin-7- | | |
| amine | | |
| copovidone | film forming agent | 112.92 |
| HPMC E3 | film forming agent | 13.48 |
| glycerol | plasticizer | 6.48 |
| water | solvent | 184.44 |
| citric acid | solubilizing agent | 42.68 |

About 75% of the total amount of water were added into a manufacturing vessel, followed by citric acid and copovidone. The citric acid and the copovidine were dissolved by stirring the solution at 400-800 RPM using an overhead stirrer for about 20 minutes. HPMC E3 was added to the remaining 25% of water in a separate vial and was dissolved by stirring at 400-500 RMP at 80° C. for about 2 hours. The active ingredient (3-(1,3-dimethyl-1H-indazol-5-yl)-2,5-dimethyl-N-[(2-methylpyridin-4-yl)methyl]pyrazolo[1,5-a]pyrimidin-7-amine) was added to the citric acid and copovidone solution and stirred for 20 min, followed by addition of the HPMC E3 solution, and then by addition of glycerol. The obtained mixture was stirred until a uniform bulk solution was obtained. The volume of the bulk solution was made up by adding the remaining 25% of water and the bulk solution was poured onto a PET membrane on the film coating machine and spread slowly at 25° C. The film was kept at 40° C. in the oven for about 1 hour and exposed to 25° C./60-65% RH for about 2 hours. A brown, translucent film was obtained, having no visible particles.

The mean thickness of the film was determined as an average of 5 thickness values obtained by placing the film in between the jaws of Vernier calipers at 5 different positions of the film. The film had a thickness of about 0.20 mm. (not less than 0.15 mm to not more than 0.25 mm).

The mean water content of the film was determined by conventional Karl Fischer (KF) titration using two samples of the film. The mean water content was determined to be lower than 10% w/w.

The dissolution rate of the film was tested by the so-called basket method (USP type-1) using a phosphate buffer (pH 6.80) as dissolution medium. A film sample was placed in the basket and the basket was immersed in 500 mL of the medium at 37° C.±0.5° C., whereafter the amount of active ingredient dissolved in the medium was determined by isocratic reversed phase high-performance liquid chromatography (HPLC) with UV detection at 217 nm. Details of the method are summarized in Table 6 herein below.

TABLE 6

| Parameter | Value |
|---|---|
| Mobile phase composition | Acetonitrile:10 mM ammonium carbonate |
| | (aq.) 35:65% v/v |
| Detector | Detector UV 217 nm |
| Column | X-Bridge C18 150 × 4.6 mm, 3.5 μm |
| Column temperature | Ambient |
| Sample tray temperature | Ambient |
| Elution mode | Isocratic |
| Flow rate | 1.2 ml/min |
| Injection volume: | 20 μL |
| Length of method | 12 minutes |
| Retention time: | Approx. 7 minutes for the active ingredient |

It was found that at least 60% of the active ingredient had dissolved in 30 minutes.

### Example 6. Mucoadhesive Film Formulation

The ingredients listed in Table 7 were used in the indicated amounts to prepare a mucoadhesive film formulation as described herein below.

TABLE 7

| | | Quantity |
|---|---|---|
| Ingredient | Function | (g) |
| 3-(1,3-dimethyl-1H-indazol-5-yl)-2,5- | active ingredient | 8.70 |
| dimethyl-N-[(2-methylpyridin-4- | | |
| yl)methyl]pyrazolo[1,5-a]pyrimidin-7- | | |
| amine | | |
| copovidone | film forming agent | 27.65 |
| HPMC E3 | film forming agent | 3.21 |
| glycerol | plasticizer | 1.55 |
| water | solvent | 48.74 |
| citric acid | solubilizing agent | 10.15 |

Water and film forming polymers were admixed in a vessel and stirred at 400 RPM for approximately 1 hour. Once the polymers had dissolved completely, citric acid was added followed by the active ingredient, and the mixture was stirred for approximately 2 hours. Finally, glycerol was added to the obtained solution, and stirring was continued until a uniform bulk solution was obtained. The bulk solution was then poured on a PET membrane placed over a film coating machine (Labkart, model LKS-C250) and spread slowly at 25° C. The final pH and viscosity of the bulk solution was found to be 3.30 and 4672 mPas, respectively. The film was then kept at 40° C. for drying in an oven for approximately 1 hour and exposed to 25° C./50-55% RH for approximately 1 hour. A brown, translucent film was obtained, having a thickness of 0.15 mm. The film had a water content of 9.2% w/w according to the Karl Fischer method. The content of active ingredient in 100 mg of the film was 12.57 mg (84% of theoretical value).

### Example 7. Comparative Single Dose Pharmacokinetic Study of Buccal Administration Vs. Oral Gavage in Male New Zealand White Rabbits

A comparative single dose pharmacokinetic study was performed wherein transmucosal systemic delivery using the film formulations of Examples 1-4 was compared to oral (enteral) systemic delivery by oral gavage of an aqueous suspension of 3-(1,3-dimethyl-1H-indazol-5-yl)-2,5-dimethyl-N-[(2-methylpyridin-4-yl)methyl]pyrazolo[1,5-a]pyrimidin-7-amine. In each case, a dose of 10 mg/kg body weight was given.

Male New Zealand White rabbits aged ~2-3 months (at the time of treatment) were used for experimentation after a minimum 7 days of acclimatization. Rabbits were weighed and anesthetized by intramuscular injection of ketamine HCl (40 mg/kg b.w.) and xylazine (10 mg/kg b.w.). Dry films containing the active ingredient were moistened with 30 μL of simulated saliva of pH 6.75 and applied to the buccal pouch of the anesthetized rabbits. The animals were in an anesthetized condition for 30 min post dose.

The animals were restrained in rabbit restrainers and blood samples (400-500 μL/time point) were collected via central ear artery at pre-dose, 0.25, 0.50, 1.0, 2.0, 4.0, 6.0, 8.0 and 24.0 hours post-dose. Collected blood specimens were centrifuged at 6000 rpm, 4° C. for 10 minutes and plasma samples were separated and stored at −80° C. until analysis. Animals did not show any adverse clinical signs up to 24 h post dose. The bioanalytical results are summarized in Table 8.

TABLE 8

| | Mean plasma pharmacokinetic parameter of active ingredient | | | | |
|---|---|---|---|---|---|
| | | | | | Enteral |
| | | | | | systemic adm. |
| PK | Transmucosal systemic administration | | | | Aqueous |
| parameter | Ex. 1 | Ex. 2 | Ex. 3 | Ex. 4 | suspension |
| Cₘₐₓ (ng/mL) | 3881.83 ± | 3770.14 ± | 2539.38 ± | 2354.41 ± | 4289.08 ± |
| | 394.55 | 719.38 | 322.09 | 894.61 | 1195.67 |
| Tₘₐₓ (h) | 4.00 ± | 5.33 ± | 4.00 ± | 6.00 ± | 1.67 ± |
| | 0.00 | 1.16 | 0.00 | 0.00 | 0.58 |
| AUCₗₐₛₜ | 47735.79 ± | 49557.52 ± | 31963.49 ± | 29547.79 ± | 49672.41 ± |
| (h*ng/mL) | 6538.18 | 10789.24 | 1139.00 | 12065.99 | 8606.98 |
| AUC_{inf} | 63907.74 ± | 94744.63 ± | 41632.07 ± | 36260.22 ± | 61263.73 ± |
| (h*ng/mL) | 15499.40 | 20194.64 | 4170.55 | 11388.78 | 15805.07 |
| AUCₑₓₜᵣₐₚ (%) | 23.98 ± | 47.72 ± | 22.85 ± | 19.90 ± | 17.70 ± |
| | 8.23 | 0.84 | 5.73 | 10.97 | 8.94 |
| T_{1/2} (h) | 10.84 ± | 19.20 ± | 10.71 ± | 9.94 ± | 9.54 ± |
| | 2.62 | 0.84 | 1.64 | 3.81 | 2.74 |
| MRTlast (h) | 9.23 ± | 11.40 ± | 8.99 ± | 9.19 ± | 7.69 ± |
| | 0.57 | 0.00 | 0.56 | 0.85 | 0.97 |

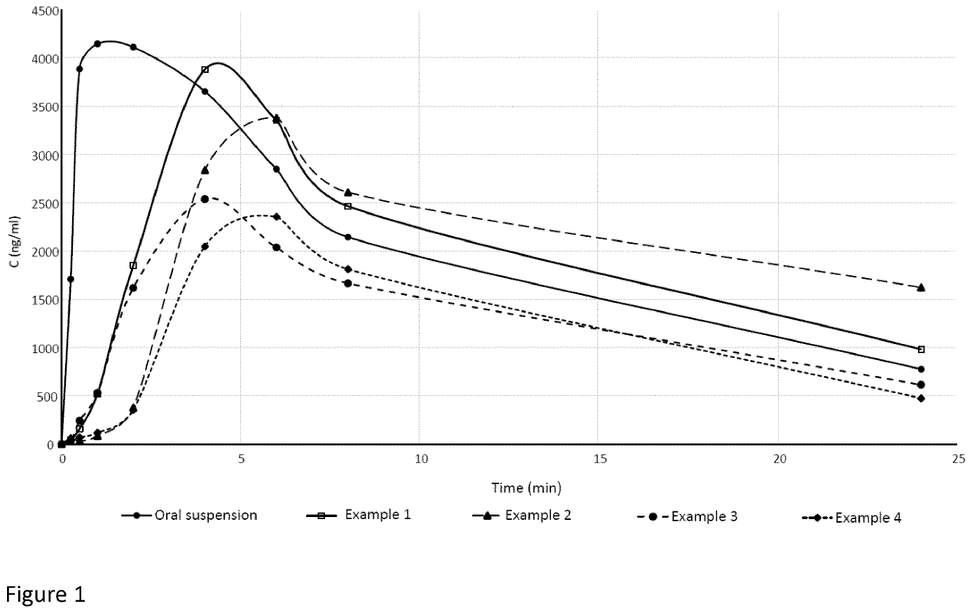

As illustrated in FIG. 1, transmucosal administration results in a lower Cₘₐₓ, and higher Tₘₐₓ compared to administration with an oral suspension. The exposure over time (AUC) and T_{1/2} are similar to the results with the oral suspension.

### Example 8. Comparative Single Dose Pharmacokinetic Study of Buccal Administration of Active Ingredient at Two Different Doses in Male New Zealand White Rabbits

Two groups of male New Zealand White rabbits aged 3-4 months (3 animals/group) were used for experimentation after 7 days of acclimatization. The rabbits were weighed (body weight: 2134.28 to 2323.42) and anesthetized by subcutaneous injection of ketamine HCl (40 mg/kg b.w.) and xylazine (10 mg/kg b.w.). Samples of the dry mucoadhesive films of Example 6 were applied to the buccal pouch of the anesthetized rabbits. After application of film sample to the buccal pouch, the film was moistened with ~100 μL of phosphate buffer of pH 6.7. The animals were in anesthetized condition for ~45 minutes post dose.

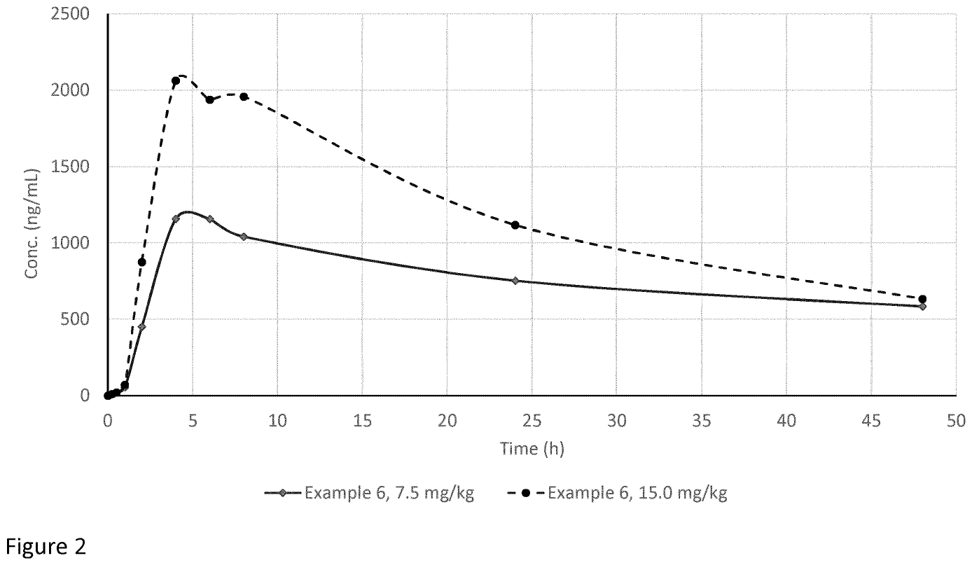

Animal were restrained in a rabbit restrainer and blood samples (400-500 μL/time point) were collected via central ear artery at pre-dose, 0.25, 0.50, 1.0, 2.0, 4.0, 6.0, 8.0, 24.0 and 48.0 hours post-dose. Collected blood specimens were centrifuged at 6000 rpm, 4° C. for 10 minutes and plasma samples were separated and stored at −80° C. until analysis. All animals were observed for any apparent signs of toxicity related to the administered dose of active ingredient during the study period. Animals were observed for up to 96 h post dose. Then animals were euthanized by overdose of sodium thiopental injection, whereafter gross observation of oral mucosa, intestines and the lymph nodes closest to the place of the films was performed to see if there was any adverse effect. The bioanalytical results are summarized in Table 9 and illustrated in FIG. 2.

TABLE 9

| | Results at 7.5 | Results at 15 |
|---|---|---|
| | mg/kg body | mg/kg body |
| Pharmacokinetic | weight dosage | weight dosage |
| parameter | by film of Ex. 6 | by film of Ex. 6 |
| Cₘₐₓ (ng/mL) | 1259.43 ± 310.33 | 2239.08 ± 795.52 |
| Tₘₐₓ (h) | 6.00 ± 2.00 | 5.33 ± 2.31 |
| AUCₗₐₛₜ (h*ng/mL) | 36786.42 ± 2355.93 | 56968.83 ± 22894.39 |
| AUC_{inf} (h*ng/mL) | Not calculated | 80522.49 ± 34721.77 |
| AUCₑₓₜᵣₐₚ (%) | Not calculated | 28.40 ± 4.85 |
| T_{1/2} (h) | Not calculated | 24.86 ± 3.44 |
| MRTlast (h) | 21.55 ± 3.69 | 18.96 ± 0.56 |

The increase in dose from 7.5 to 15 mg/kg b.w. resulted in an 1.8 and 1.5 increase of the plasma maximum concentration (Cₘₐₓ) and plasma exposure (AUC₀₋₄₈ₕ). No adverse clinical signs were observed in any of the treated rabbits during the study period. Gross observation of oral mucosa, intestines and the lymph nodes performed at 96 hours post dose and showed no external or internal gross pathological changes.

### Example 9. Mucoadhesive Film Formulation

A mucoadhesive film formulation was prepared by following essentially the procedure of Example 6, using the ingredients listed in Table 10.

TABLE 10

| | | Quantity |
|---|---|---|
| Ingredient | Function | (% w/w) |
| 3-(1,3-dimethyl-1H-indazol-5-yl)-2,5- | active ingredient | 8.3 |
| dimethyl-N-[(2-methylpyridin-4- | | |
| yl)methyl]pyrazolo[1,5-a]pyrimidin-7- | | |
| amine | | |
| copovidone | film forming agent | 24.1 |
| glycerol | plasticizer | 2.8 |
| water | solvent | 55.5 |
| citric acid | solubilizing agent | 9.3 |

A clear, slightly brittle, dry film (KF water content of about 9%) was obtained containing approximately 15 mg of active ingredient/100 mg dry film.

### Example 10. Mucoadhesive Film Formulation

A mucoadhesive film formulation was prepared by following essentially the procedure of Example 6, using the ingredients listed in Table 11.

TABLE 11

| | | Quantity |
|---|---|---|
| Ingredient | Function | (% w/w) |
| 3-(1,3-dimethyl-1H-indazol-5-yl)-2,5- | active ingredient | 8.3 |
| dimethyl-N-[(2-methylpyridin-4- | | |
| yl)methyl]pyrazolo[1,5-a]pyrimidin-7- | | |
| amine | | |
| copovidone | film forming agent | 20.5 |
| HPMC E3 | film forming agent | 3.6 |
| glycerol | plasticizer | 2.8 |
| water | solvent | 55.6 |
| citric acid | solubilizing agent | 9.2 |

A slightly opaque but flexible film (KF water content of about 9%), was obtained containing approximately 15 mg of active ingredient/100 mg dry film.

### Example 11. Mucoadhesive Film Formulation

A mucoadhesive film formulation was prepared by following essentially the procedure of Example 6, using the ingredients listed in Table 12.

TABLE 12

| | | Quantity |
|---|---|---|
| Ingredient | Function | (% w/w) |
| 3-(1,3-dimethyl-1H-indazol-5-yl)-2,5- | active ingredient | 8.3 |
| dimethyl-N-[(2-methylpyridin-4- | | |
| yl)methyl]pyrazolo[1,5-a]pyrimidin-7- | | |
| amine | | |
| copovidone | film forming agent | 21.6 |
| HPMC E3 | film forming agent | 2.5 |
| glycerol | plasticizer | 2.8 |
| water | solvent | 55.6 |
| citric acid | solubilizing agent | 9.2 |

A slightly opaque but flexible film (KF water content of about 9%), was obtained containing approximately 15 mg of active ingredient/100 mg dry film.

### Example 12. Mucoadhesive Film Formulation

A mucoadhesive film formulation was prepared by following essentially the procedure of Example 4, using the ingredients listed in Table 13.

TABLE 13

| | | Quantity |
|---|---|---|
| Ingredient | Function | (% w/w) |
| 3-(1,3-dimethyl-1H-indazol-5-yl)-2,5- | active ingredient | 8.3 |
| dimethyl-N-[(2-methylpyridin-4- | | |
| yl)methyl]pyrazolo[1,5-a]pyrimidin-7- | | |
| amine | | |
| copovidone | film forming agent | 21.7 |
| HPMC E3 | film forming agent | 2.5 |
| Tween ® 80 | surfactant | 0.9 |
| glycerol | plasticizer | 1.3 |
| water | solvent | 56.1 |
| citric acid | solubilizing agent | 9.2 |

A highly flexible, opaque dry film (water content of about 11.44%), was obtained containing approximately 16 mg of active ingredient/100 mg dry film (85.1% of theoretical value).

### Example 13. Mucoadhesive Film Formulation

A mucoadhesive film formulation was prepared by following essentially the procedure of Example 6, using the ingredients listed in Table 14.

TABLE 14

| | | Quantity |
|---|---|---|
| Ingredient | Function | (% w/w) |
| 3-(1,3-dimethyl-1H-indazol-5-yl)-2,5- | active ingredient | 8.5 |
| dimethyl-N-[(2-methylpyridin-4- | | |
| yl)methyl]pyrazolo[1,5-a]pyrimidin-7- | | |
| amine | | |
| copovidone | film forming agent | 22.0 |
| HPMC E3 | film forming agent | 2.5 |
| glycerol | plasticizer | 1.4 |
| water | solvent | 56.3 |
| citric acid | solubilizing agent | 9.3 |

A flexible, slightly opaque dry film was obtained, having good strength, having a KF water content of 10.56 and containing approximately 15.5 mg of active ingredient/100 mg dry film (80.7% of theoretical value).

The dissolution profile of the film of Example 13 was tested essentially as described for the film of Example 5. The sample size was 83.17 mg, which was dissolved in 200 mL of pH 7.4 phosphate buffer at 50 RPM. Within 30 minutes, 60% of the active ingredient had dissolved.

### Example 14. Inhibition of PI4 Kinase

Inhibition of PI4 kinases was performed with the ADP-Glo™ Kinase assay which measures the ADP formed from a kinase reaction, where ADP is converted into ATP and then converted into light by Ultra-Glo™ Luciferase. Reactions were carried out for 60 min at 10 μM ATP. The luminescent signal produced in reaction is proportional to the kinase activity.

Inhibition of PI3 kinases was studied using the HTRF (homogeneous time-resolved fluorescence) assay which is a universal method for identifying and characterizing the phosphotransferase activity induced by any ATP/ADP dependent target. The formation of ADP was detected by a specific monoclonal antibody labeled with Eu³⁺ cryptate; the amount of ADP formed directly correlates with the amount of phosphorylated substrate in kinase assays.

IC₅₀ values measured for the compound 3-(1,3-dimethyl-1H-indazol-5-yl)-2,5-dimethyl-N-[(2-methylpyridin-4-yl)methyl]pyrazolo[1,5-a]pyrimidin-7-amine are shown in Table 15.

TABLE 15

| Kinase | IC₅₀ | |
|---|---|---|
| PI3KA (p110a/p85a) | 3.4 | μM |
| PI3KB (p110b/p85a) | 5.4 | μM |
| PI3K (p110g/p85a) | >1 | μM* |
| PI3K (p120g) | >1 | μM* |
| PI4K2A | >10 | μM** |
| PI4K2B | >10 | μM** |
| PI4KA | 1.1 | μM |
| PI4KB | 0.6 | nM |

*Not tested at concentration higher than 1 μM.
**Not tested at concentration higher than 10 μM.

The obtained IC₅₀ values indicate a very potent PI4 KB inhibition with a selectivity of >1000 times to the closest related kinase PI4KA. Generally, compounds of formula (I), as defined herein, are highly potent inhibitors of PI4 KB with IC₅₀-values between 1 and 2 nM; less potent inhibitors of PI4KA, with IC₅₀-values between 1 and 3 μM; and showing low or negligible inhibition of PI3KA and PI3 KB, with IC₅₀-values higher than 10 μM. This indicates that the compounds are highly selective PI4 KB inhibitors.

1.-6. (canceled)

7. A pharmaceutical formulation for oral transmucosal systemic administration of a compound of formula (I) 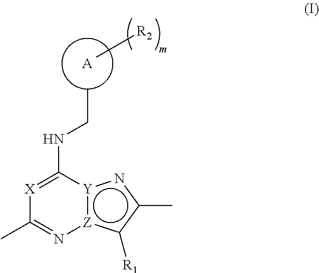
or a pharmaceutically acceptable salt thereof, wherein
X is CH, Y is C, and Z is N, or
X is CH, Y is N, and Z is C, or
X is N, Y is N, and Z is C;
R₁ is 3,4-dimethoxyphenyl or 1,3-dimethyl-1H-indazol-5-yl;
ring A is phenyl, or 5- or 6-membered heteroaryl;
m is 0, 1, 2 or 3;
each R₂ is independently selected from C₁-C₆ alkyl, halogen, R₃O, R₄R₅N, R₆R₇NS(O)₂, R₈R₉NC(O), R₁₀S(O)₂, R₁₁S(O)₂N(R₁₂), R₁₃C(O)O, R₁₄C(O)N(R₁₅), R₁₆C(O), R₁₇C(O), R₁₈C(O)N(R₁₉)S(O)₂, R₂₀OC(O)N(R₂₁)S(O)₂, CN and —O—; and two R₃, attached to adjacent atoms of ring A, together with the atoms to which they are attached may form a 5- or 6-membered heterocyclic or carbocyclic ring;
each R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ is independently selected from H and C₁-C₆ alkyl; and
any alkyl is optionally substituted by one or more F.

8. The pharmaceutical formulation according to claim 7, wherein
X is CH, Y is C, and Z is N, or X is CH, Y is N, and Z is C;
ring A is phenyl, or 6-membered heteroaryl; and
R₁ is 1,3-dimethyl-1H-indazol-5-yl.

9. The pharmaceutical formulation according to claim 7, wherein the compound is selected from
3-(3,4-dimethoxyphenyl)-2,6-dimethyl-N-(pyridin-4-ylmethyl)imidazo[1,2-b]pyridazin-8-amine,
3-(3,4-dimethoxyphenyl)-2,6-dimethyl-N-((2-methylpyridin-4-yl)methyl)imidazo[1,2-b]pyridazin-8-amine,
3-(3,4-dimethoxyphenyl)-N-((2-fluoropyridin-4-yl)methyl)-2,6-dimethylimidazo[1,2-b]pyridazin-8-amine,
3-(1,3-dimethyl-1H-indazol-5-yl)-2,5-dimethyl-N-[(2-fluoropyridin-4-yl)methyl]pyrazolo[1,5-a]pyrimidin-7-amine,
3-(1,3-dimethyl-1H-indazol-5-yl)-2,5-dimethyl-N-[(2-methylpyridin-4-yl)methyl]pyrazolo[1,5-a]pyrimidin-7-amine,
3-(1,3-dimethyl-1H-indazol-5-yl)-2,5-dimethyl-N-[(2-ethylpyridin-4-yl)methyl]pyrazolo[1,5-a]pyrimidin-7-amine,
3-(1,3-dimethyl-1H-indazol-5-yl)-2,5-dimethyl-N-[(2-methoxypyridin-4-yl)methyl]pyrazolo[1,5-a]pyrimidin-7-amine,
3-(1,3-dimethyl-1H-indazol-5-yl)-2,5-dimethyl-N-[(2-cyanopyridin-4-yl)methyl]pyrazolo[1,5-a]pyrimidin-7-amine,
3-(1,3-dimethyl-1H-indazol-5-yl)-2,5-dimethyl-N-[(2-trifluoromethylpyridin-4-yl)methyl]pyrazolo[1,5-a]pyrimidin-7-amine,
N-[(4-chlorophenyl)methyl]-3-(3,4-dimethoxyphenyl)-2,5-dimethyl-pyrazolo[1,5-a]pyrimidin-7-amine,
3-(3,4-dimethoxyphenyl)-N-[(4-methoxyphenyl)methyl]-2,5-dimethyl-pyrazolo[1,5-a]pyrimidin-7-amine,
3-(3,4-dimethoxyphenyl)-2,5-dimethyl-N-(p-tolylmethyl)pyrazolo[1,5-a]pyrimidin-7-amine,
N-(1,3-benzodioxol-5-ylmethyl)-3-(3,4-dimethoxyphenyl)-2,5-dimethyl-pyrazolo[1,5-a]pyrimidin-7-amine,
3-(3,4-dimethoxyphenyl)-N-[(4-fluorophenyl)methyl]-2,5-dimethyl-pyrazolo[1,5-a]pyrimidin-7-amine,
3-(3,4-dimethoxyphenyl)-2,5-dimethyl-N-(2-pyridylmethyl)pyrazolo[1,5-a]pyrimidin-7-amine,
N-benzyl-3-(3,4-dimethoxyphenyl)-2,5-dimethyl-pyrazolo[1,5-a]pyrimidin-7-amine,
3-(3,4-dimethoxyphenyl)-2,5-dimethyl-N-[[4-(trifluoromethyl)phenyl]methyl]pyrazolo[1,5-a]pyrimidin-7-amine,
3-(3,4-dimethoxyphenyl)-2,5-dimethyl-N-(4-pyridylmethyl)pyrazolo[1,5-a]pyrimidin-7-amine,
3-(3,4-dimethoxyphenyl)-2,5-dimethyl-N-[[4-(trifluoromethoxy)phenyl]methyl]pyrazolo[1,5-a]pyrimidin-7-amine,
N-[4-[[[3-(3,4-dimethoxyphenyl)-2,5-dimethyl-pyrazolo[1,5-a]pyrimidin-7-yl]amino]methyl]phenyl]acetamide,
3-(3,4-dimethoxyphenyl)-N-[(4-dimethylaminophenyl)methyl]-2,5-dimethyl-pyrazolo[1,5-a]pyrimidin-7-amine,
3-(3,4-dimethoxyphenyl)-N-[(6-methoxy-3-pyridyl)methyl]-2,5-dimethyl-pyrazolo[1,5-a]pyrimidin-7-amine,
N-[4-[[[3-(3,4-dimethoxyphenyl)-2,5-dimethyl-pyrazolo[1,5-a]pyrimidin-7-yl]amino]methyl]phenyl]methanesulfonamide,
4-[[[3-(3,4-dimethoxyphenyl)-2,5-dimethyl-pyrazolo[1,5-a]pyrimidin-7-yl]amino]methyl]phenol,
3-(3,4-dimethoxyphenyl)-2,5-dimethyl-N-[(3-methylsulfonylphenyl)methyl]pyrazolo[1,5-a]pyrimidin-7-amine,
3-(3,4-dimethoxyphenyl)-2,5-dimethyl-N-[(4-methylsulfonylphenyl)methyl]pyrazolo[1,5-a]pyrimidin-7-amine,
N-[(4-tert-butylphenyl)methyl]-3-(3,4-dimethoxyphenyl)-2,5-dimethyl-pyrazolo[1,5-a]pyrimidin-7-amine,
3-(3,4-dimethoxyphenyl)-2,5-dimethyl-N-[(2-methylpyrimidin-4-yl)methyl]pyrazolo[1,5-a]pyrimidin-7-amine,
3-(3,4-dimethoxyphenyl)-2,5-dimethyl-N-[(6-methyl-3-pyridyl)methyl]pyrazolo[1,5-a]pyrimidin-7-amine,
1-[4-[[[3-(3,4-dimethoxyphenyl)-2,5-dimethyl-pyrazolo[1,5-a]pyrimidin-7-yl]amino]methyl]phenyl]ethanone,
3-(3,4-dimethoxyphenyl)-2,5-dimethyl-N-(1-naphthylmethyl)pyrazolo[1,5-a]pyrimidin-7-amine,
4-[[[3-(3,4-dimethoxyphenyl)-2,5-dimethyl-pyrazolo[1,5-a]pyrimidin-7-yl]amino]methyl]benzenesulfonamide,
8-(3,4-dimethoxyphenyl)-N-[(4-fluorophenyl)methyl]-2,7-dimethyl-pyrazolo[1,5-a][1,3,5]triazin-4-amine,
8-(3,4-dimethoxyphenyl)-2,7-dimethyl-N-[[4-(trifluoromethyl)phenyl]methyl]pyrazolo[1,5-a][1,3,5]triazin-4-amine,
8-(3,4-dimethoxyphenyl)-2,7-dimethyl-N-(p-tolylmethyl)pyrazolo[1,5-a][1,3,5]triazin-4-amine,
8-(3,4-dimethoxyphenyl)-N-[(4-isopropylphenyl)methyl]-2,7-dimethyl-pyrazolo[1,5-a][1,3,5]triazin-4-amine,
8-(3,4-dimethoxyphenyl)-2,7-dimethyl-N-(2-pyridylmethyl)pyrazolo[1,5-a][1,3,5]triazin-4-amine,
8-(3,4-dimethoxyphenyl)-2,7-dimethyl-N-(4-pyridylmethyl)pyrazolo[1,5-a][1,3,5]triazin-4-amine,
N-[(4-chlorophenyl)methyl]-8-(3,4-dimethoxyphenyl)-2,7-dimethyl-pyrazolo[1,5-a][1,3,5]triazin-4-amine,
N-[4-[[[8-(3,4-dimethoxyphenyl)-2,7-dimethyl-pyrazolo[1,5-a][1,3,5]triazin-4-yl]amino]methyl]phenyl]acetamide,
8-(3,4-dimethoxyphenyl)-N-[(4-methoxyphenyl)methyl]-2,7-dimethyl-pyrazolo[1,5-a][1,3,5]triazin-4-amine,
8-(3,4-dimethoxyphenyl)-2,7-dimethyl-N-[(3-methylsulfonylphenyl)methyl]pyrazolo[1,5-a][1,3,5]triazin-4-amine,
8-(3,4-dimethoxyphenyl)-2,7-dimethyl-N-[(1-oxidopyridin-1-ium-4-yl)methyl]pyrazolo[1,5-a][1,3,5]triazin-4-amine,
8-(3,4-dimethoxyphenyl)-N-[(6-methoxy-3-pyridyl)methyl]-2,7-dimethyl-pyrazolo[1,5-a][1,3,5]triazin-4-amine,
4-[[[8-(3,4-dimethoxyphenyl)-2,7-dimethyl-pyrazolo[1,5-a][1,3,5]triazin-4-yl]amino]methyl]benzenesulfonamide,
N-(1,3-benzodioxol-5-ylmethyl)-8-(3,4-dimethoxyphenyl)-2,7-dimethyl-pyrazolo[1,5-a][1,3,5]triazin-4-amine,
4-[[[8-(3,4-dimethoxyphenyl)-2,7-dimethyl-pyrazolo[1,5-a][1,3,5]triazin-4-yl]amino]methyl]phenol,
N-[4-[[[8-(3,4-dimethoxyphenyl)-2,7-dimethyl-pyrazolo[1,5-a][1,3,5]triazin-4-yl]amino]methyl]phenyl]methanesulfonamide,
N-benzyl-8-(3,4-dimethoxyphenyl)-2,7-dimethyl-pyrazolo[1,5-a][1,3,5]triazin-4-amine,
8-(3,4-dimethoxyphenyl)-2,7-dimethyl-N-(2-thienylmethyl)pyrazolo[1,5-a][1,3,5]triazin-4-amine,
8-(3,4-dimethoxyphenyl)-2,7-dimethyl-N-(3-pyridylmethyl)pyrazolo[1,5-a][1,3,5]triazin-4-amine,
8-(3,4-dimethoxyphenyl)-2,7-dimethyl-N-[(5-methyl-2-furyl)methyl]pyrazolo[1,5-a][1,3,5]triazin-4-amine,
methyl N-[4-[[[8-(3,4-dimethoxyphenyl)-2,7-dimethyl-pyrazolo[1,5-a][1,3,5]triazin-4-yl]amino]methyl]phenyl]sulfonylcarbamate,
N-[4-[[[8-(3,4-dimethoxyphenyl)-2,7-dimethyl-pyrazolo[1,5-a][1,3,5]triazin-4-yl]amino]methyl]phenyl]sulfonylpropanamide,
N-methyl-4-[[[8-(3,4-dimethoxyphenyl)-2,7-dimethyl-pyrazolo[1,5-a][1,3,5]triazin-4-yl]amino]methyl]benzenesulfonamide,
8-(3,4-dimethoxyphenyl)-2,7-dimethyl-N-[(1-methyl-1H-pyrazol-3-yl)methyl]pyrazolo[1,5-a][1,3,5]triazin-4-amine,
8-(3,4-dimethoxyphenyl)-2,7-dimethyl-N-[(1,3-oxazol-5-yl)methyl]pyrazolo[1,5-a][1,3,5]triazin-4-amine,
8-(3,4-dimethoxyphenyl)-2,7-dimethyl-N-[(2-methylpyrimidin-5-yl)methyl]pyrazolo[1,5-a][1,3,5]triazin-4-amine,
8-(3,4-dimethoxyphenyl)-2,7-dimethyl-N-[(pyridazin-4-yl)methyl]pyrazolo[1,5-a][1,3,5]triazin-4-amine,
8-(3,4-dimethoxyphenyl)-2,7-dimethyl-N-[(6-methylpyridin-3-yl)methyl]pyrazolo[1,5-a][1,3,5]triazin-4-amine,
3-(1,3-dimethyl-1H-indazol-5-yl)-2,6-dimethyl-N-(pyridin-4-ylmethyl)imidazo[1,2-b]pyridazin-8-amine,
3-(1,3-dimethyl-1H-indazol-5-yl)-2,6-dimethyl-N-((6-methylpyridin-3-yl)methyl)imidazo[1,2-b]pyridazin-8-amine,
N-(4-chlorobenzyl)-3-(1,3-dimethyl-1H-indazol-5-yl)-2,6-dimethylimidazo[1,2-b]pyridazin-8-amine,
3-(1,3-dimethyl-1H-indazol-5-yl)-N-((6-methoxypyridin-3-yl)methyl)-2,6-dimethylimidazo[1,2-b]pyridazin-8-amine,
3-(1,3-dimethyl-1H-indazol-5-yl)-2,6-dimethyl-N-(thiophen-2-ylmethyl)imidazo[1,2-b]pyridazin-8-amine,
3-(1,3-dimethyl-1H-indazol-5-yl)-N-(4-methoxybenzyl)-2,6-dimethylimidazo[1,2-b]pyridazin-8-amine,
3-(1,3-dimethyl-1H-indazol-5-yl)-2,6-dimethyl-N-(4-(methylsulfonyl)benzyl)imidazo[1,2-b]pyridazin-8-amine,
3-(1,3-dimethyl-1H-indazol-5-yl)-2,5-dimethyl-N-(pyridin-4-ylmethyl)pyrazolo[1,5-a]pyrimidin-7-amine,
3-(1,3-dimethyl-1H-indazol-5-yl)-2,5-dimethyl-N-((6-methylpyridin-3-yl)methyl)pyrazolo[1,5-a]pyrimidin-7-amine,
3-(1,3-dimethyl-1H-indazol-5-yl)-N-(4-methoxybenzyl)-2,5-dimethylpyrazolo[1,5-a]pyrimidin-7-amine,
N-(4-chlorobenzyl)-3-(1,3-dimethyl-1H-indazol-5-yl)-2,5-dimethylpyrazolo[1,5-a]pyrimidin-7-amine,
3-(1,3-dimethyl-1H-indazol-5-yl)-2,5-dimethyl-N-(4-(methylsulfonyl)benzyl)pyrazolo[1,5-a]pyrimidin-7-amine,
4-(((3-(1,3-dimethyl-1H-indazol-5-yl)-2,5-dimethylpyrazolo[1,5-a]pyrimidin-7-yl)amino)methyl)benzenesulfonamide,
3-(1,3-dimethyl-1H-indazol-5-yl)-N-((6-methoxypyridin-3-yl)methyl)-2,5-dimethylpyrazolo[1,5-a]pyrimidin-7-amine,
3-(1,3-dimethyl-1H-indazol-5-yl)-2,5-dimethyl-N-(thiophen-2-ylmethyl)pyrazolo[1,5-a]pyrimidin-7-amine,
N-(4-(((3-(1,3-dimethyl-1H-indazol-5-yl)-2,5-dimethylpyrazolo[1,5-a]pyrimidin-7-yl)amino)methyl)phenyl)methanesulfonamide,
3-(1,3-dimethyl-1H-indazol-5-yl)-2,5-dimethyl-N-((6-trifluoromethylpyridin-3-yl)methyl)pyrazolo[1,5-a]pyrimidin-7-amine,
N-(benzyl)-3-(1,3-dimethyl-1H-indazol-5-yl)-2,5-dimethylpyrazolo[1,5-a]pyrimidin-7-amine,
N-(4-fluorobenzyl)-3-(1,3-dimethyl-1H-indazol-5-yl)-2,5-dimethylpyrazolo[1,5-a]pyrimidin-7-amine,
8-(1,3-dimethyl-1H-indazol-5-yl)-2,7-dimethyl-N-(pyridin-4-ylmethyl)pyrazolo[1,5-a][1,3,5]triazin-4-amine,
8-(1,3-dimethyl-1H-indazol-5-yl)-N-(4-methoxybenzyl)-2,7-dimethylpyrazolo[1,5-a][1,3,5]triazin-4-amine,
8-(1,3-dimethyl-1H-indazol-5-yl)-2,7-dimethyl-N-((6-methylpyridin-3-yl)methyl)pyrazolo[1,5-a][1,3,5]triazin-4-amine,
4-(((8-(1,3-dimethyl-1H-indazol-5-yl)-2,7-dimethylpyrazolo[1,5-a][1,3,5]triazin-4-yl)amino)methyl)benzenesulfonamide,
N-(4-chlorobenzyl)-8-(1,3-dimethyl-1H-indazol-5-yl)-2,7-dimethylpyrazolo[1,5-a][1,3,5]triazin-4-amine,
8-(1,3-dimethyl-1H-indazol-5-yl)-2,7-dimethyl-N-(4-(methylsulfonyl)benzyl)pyrazolo[1,5-a][1,3,5]triazin-4-amine,
N-(4-(((8-(1,3-dimethyl-1H-indazol-5-yl)-2,7-dimethylpyrazolo[1,5-a][1,3,5]triazin-4-yl)amino)methyl)phenyl)methanesulfonamide,
8-(1,3-dimethyl-1H-indazol-5-yl)-2,7-dimethyl-N-(thiophen-2-ylmethyl)pyrazolo[1,5-a][1,3,5]triazin-4-amine, and
8-(1,3-dimethyl-1H-indazol-5-yl)-N-((6-methoxypyridin-3-yl)methyl)-2,7-dimethylpyrazolo[1,5-a][1,3,5]triazin-4-amine.

10. The pharmaceutical formulation according to claim 7, wherein the oral transmucosal administration is buccal transmucosal administration.

11. The pharmaceutical formulation according to claim 7, in the form of a mucoadhesive polymeric film comprising the compound of formula (I) or a pharmaceutically acceptable salt thereof.

12. The pharmaceutical formulation according to claim 7, further comprising one or more excipients selected from plasticizers, solubilizers, taste-masking agents, colorants, antioxidation agents, preservatives, penetration enhancers, and mucoadhesive agents.

13. The pharmaceutical formulation according to claim 7, wherein the compound of formula (I) or the pharmaceutically acceptable salt thereof is present in the formulation in an amount of from 1 to 50% by weight, relative to the total dry weight of the formulation.

14.-17. (canceled)

18. A method of systemic treatment of a virus infection or a disorder for which PI4K inhibition is beneficial, selected from pancreatitis, poliomyelitis, encephalitis, meningitis, sepsis, cancer, paralysis, cardiac diseases, diabetes, common cold, hand-foot-and-mouth disease, herpangina, pleurodynia, diarrhoea, mucocutaneous lesions, respiratory illness, conjunctivitis, myositis, chronic fatigue syndrome, neuropsychiatric diseases, neurodegenerative diseases, infectious diseases, and inflammatory conditions, by oral transmucosal administration, to a mammal in need of such treatment, of a compound of formula (I) 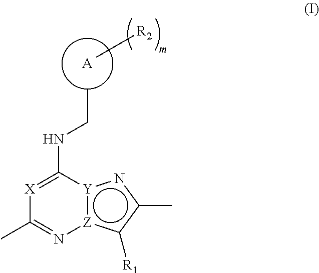
or a pharmaceutically acceptable salt thereof, wherein
X is CH, Y is C, and Z is N, or
X is CH, Y is N, and Z is C, or
X is N, Y is N, and Z is C;
R₁ is 3,4-dimethoxyphenyl or 1,3-dimethyl-1H-indazol-5-yl;
ring A is phenyl, or 5- or 6-membered heteroaryl;
m is 0, 1, 2 or 3;
each R₂ is independently selected from C₁-C₆ alkyl, halogen, R₃O, R₄R₅N, R₆R₇NS(O)₂, R₈R₉NC(O), R₁₀S(O)₂, R₁₁S(O)₂N(R₁₂), R₁₃C(O)O, R₁₄C(O)N(R₁₅), R₁₆C(O), R₁₇C(O), R₁₈C(O)N(R₁₉)S(O)₂, R₂₀OC(O)N(R₂₁)S(O)₂, CN and —O—; and two R₃, attached to adjacent atoms of ring A, together with the atoms to which they are attached may form a 5- or 6-membered heterocyclic or carbocyclic ring;
each R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, and R₂₁ is independently selected from H and C₁-C₆ alkyl; and
any alkyl is optionally substituted by one or more F.

19. The method of claim 18, wherein
X is CH, Y is C, and Z is N, or X is CH, Y is N, and Z is C;
ring A is phenyl, or 6-membered heteroaryl; and
R₁ is 1,3-dimethyl-1H-indazol-5-yl.

20. The method of claim 18, wherein the compound is selected from
3-(3,4-dimethoxyphenyl)-2,6-dimethyl-N-(pyridin-4-ylmethyl)imidazo[1,2-b]pyridazin-8-amine,
3-(3,4-dimethoxyphenyl)-2,6-dimethyl-N-((2-methylpyridin-4-yl)methyl)imidazo[1,2-b]pyridazin-8-amine,
3-(3,4-dimethoxyphenyl)-N-((2-fluoropyridin-4-yl)methyl)-2,6-dimethylimidazo[1,2-b]pyridazin-8-amine,
3-(1,3-dimethyl-1H-indazol-5-yl)-2,5-dimethyl-N-[(2-fluoropyridin-4-yl)methyl]pyrazolo[1,5-a]pyrimidin-7-amine,
3-(1,3-dimethyl-1H-indazol-5-yl)-2,5-dimethyl-N-[(2-methylpyridin-4-yl)methyl]pyrazolo[1,5-a]pyrimidin-7-amine,
3-(1,3-dimethyl-1H-indazol-5-yl)-2,5-dimethyl-N-[(2-ethylpyridin-4-yl)methyl]pyrazolo[1,5-a]pyrimidin-7-amine,
3-(1,3-dimethyl-1H-indazol-5-yl)-2,5-dimethyl-N-[(2-methoxypyridin-4-yl)methyl]pyrazolo[1,5-a]pyrimidin-7-amine,
3-(1,3-dimethyl-1H-indazol-5-yl)-2,5-dimethyl-N-[(2-cyanopyridin-4-yl)methyl]pyrazolo[1,5-a]pyrimidin-7-amine,
3-(1,3-dimethyl-1H-indazol-5-yl)-2,5-dimethyl-N-[(2-trifluoromethylpyridin-4-yl)methyl]pyrazolo[1,5-a]pyrimidin-7-amine,
N-[(4-chlorophenyl)methyl]-3-(3,4-dimethoxyphenyl)-2,5-dimethyl-pyrazolo[1,5-a]pyrimidin-7-amine,
3-(3,4-dimethoxyphenyl)-N-[(4-methoxyphenyl)methyl]-2,5-dimethyl-pyrazolo[1,5-a]pyrimidin-7-amine,
3-(3,4-dimethoxyphenyl)-2,5-dimethyl-N-(p-tolylmethyl)pyrazolo[1,5-a]pyrimidin-7-amine,
N-(1,3-benzodioxol-5-ylmethyl)-3-(3,4-dimethoxyphenyl)-2,5-dimethyl-pyrazolo[1,5-a]pyrimidin-7-amine,
3-(3,4-dimethoxyphenyl)-N-[(4-fluorophenyl)methyl]-2,5-dimethyl-pyrazolo[1,5-a]pyrimidin-7-amine,
3-(3,4-dimethoxyphenyl)-2,5-dimethyl-N-(2-pyridylmethyl)pyrazolo[1,5-a]pyrimidin-7-amine,
N-benzyl-3-(3,4-dimethoxyphenyl)-2,5-dimethyl-pyrazolo[1,5-a]pyrimidin-7-amine,
3-(3,4-dimethoxyphenyl)-2,5-dimethyl-N-[[4-(trifluoromethyl)phenyl]methyl]pyrazolo[1,5-a]pyrimidin-7-amine,
3-(3,4-dimethoxyphenyl)-2,5-dimethyl-N-(4-pyridylmethyl)pyrazolo[1,5-a]pyrimidin-7-amine,
3-(3,4-dimethoxyphenyl)-2,5-dimethyl-N-[[4-(trifluoromethoxy)phenyl]methyl]pyrazolo[1,5-a]pyrimidin-7-amine,
N-[4-[[[3-(3,4-dimethoxyphenyl)-2,5-dimethyl-pyrazolo[1,5-a]pyrimidin-7-yl]amino]methyl]phenyl]acetamide,
3-(3,4-dimethoxyphenyl)-N-[(4-dimethylaminophenyl)methyl]-2,5-dimethyl-pyrazolo[1,5-a]pyrimidin-7-amine,
3-(3,4-dimethoxyphenyl)-N-[(6-methoxy-3-pyridyl)methyl]-2,5-dimethyl-pyrazolo[1,5-a]pyrimidin-7-amine,
N-[4-[[[3-(3,4-dimethoxyphenyl)-2,5-dimethyl-pyrazolo[1,5-a]pyrimidin-7-yl]amino]methyl]phenyl]methanesulfonamide,
4-[[[3-(3,4-dimethoxyphenyl)-2,5-dimethyl-pyrazolo[1,5-a]pyrimidin-7-yl]amino]methyl]phenol,
3-(3,4-dimethoxyphenyl)-2,5-dimethyl-N-[(3-methylsulfonylphenyl)methyl]pyrazolo[1,5-a]pyrimidin-7-amine,
3-(3,4-dimethoxyphenyl)-2,5-dimethyl-N-[(4-methylsulfonylphenyl)methyl]pyrazolo[1,5-a]pyrimidin-7-amine,
N-[(4-tert-butylphenyl)methyl]-3-(3,4-dimethoxyphenyl)-2,5-dimethyl-pyrazolo[1,5-a]pyrimidin-7-amine,
3-(3,4-dimethoxyphenyl)-2,5-dimethyl-N-[(2-methylpyrimidin-4-yl)methyl]pyrazolo[1,5-a]pyrimidin-7-amine,
3-(3,4-dimethoxyphenyl)-2,5-dimethyl-N-[(6-methyl-3-pyridyl)methyl]pyrazolo[1,5-a]pyrimidin-7-amine,
1-[4-[[[3-(3,4-dimethoxyphenyl)-2,5-dimethyl-pyrazolo[1,5-a]pyrimidin-7-yl]amino]methyl]phenyl]ethanone,
3-(3,4-dimethoxyphenyl)-2,5-dimethyl-N-(1-naphthylmethyl)pyrazolo[1,5-a]pyrimidin-7-amine,
4-[[[3-(3,4-dimethoxyphenyl)-2,5-dimethyl-pyrazolo[1,5-a]pyrimidin-7-yl]amino]methyl]benzenesulfonamide,
8-(3,4-dimethoxyphenyl)-N-[(4-fluorophenyl)methyl]-2,7-dimethyl-pyrazolo[1,5-a][1,3,5]triazin-4-amine,
8-(3,4-dimethoxyphenyl)-2,7-dimethyl-N-[[4-(trifluoromethyl)phenyl]methyl]pyrazolo[1,5-a][1,3,5]triazin-4-amine,
8-(3,4-dimethoxyphenyl)-2,7-dimethyl-N-(p-tolylmethyl)pyrazolo[1,5-a][1,3,5]triazin-4-amine,
8-(3,4-dimethoxyphenyl)-N-[(4-isopropylphenyl)methyl]-2,7-dimethyl-pyrazolo[1,5-a][1,3,5]triazin-4-amine,
8-(3,4-dimethoxyphenyl)-2,7-dimethyl-N-(2-pyridylmethyl)pyrazolo[1,5-a][1,3,5]triazin-4-amine,
8-(3,4-dimethoxyphenyl)-2,7-dimethyl-N-(4-pyridylmethyl)pyrazolo[1,5-a][1,3,5]triazin-4-amine,
N-[(4-chlorophenyl)methyl]-8-(3,4-dimethoxyphenyl)-2,7-dimethyl-pyrazolo[1,5-a][1,3,5]triazin-4-amine,
N-[4-[[[8-(3,4-dimethoxyphenyl)-2,7-dimethyl-pyrazolo[1,5-a][1,3,5]triazin-4-yl]amino]methyl]phenyl]acetamide,
8-(3,4-dimethoxyphenyl)-N-[(4-methoxyphenyl)methyl]-2,7-dimethyl-pyrazolo[1,5-a][1,3,5]triazin-4-amine,
8-(3,4-dimethoxyphenyl)-2,7-dimethyl-N-[(3-methylsulfonylphenyl)methyl]pyrazolo[1,5-a][1,3,5]triazin-4-amine,
8-(3,4-dimethoxyphenyl)-2,7-dimethyl-N-[(1-oxidopyridin-1-ium-4-yl)methyl]pyrazolo[1,5-a][1,3,5]triazin-4-amine,
8-(3,4-dimethoxyphenyl)-N-[(6-methoxy-3-pyridyl)methyl]-2,7-dimethyl-pyrazolo[1,5-a][1,3,5]triazin-4-amine,
4-[[[8-(3,4-dimethoxyphenyl)-2,7-dimethyl-pyrazolo[1,5-a][1,3,5]triazin-4-yl]amino]methyl]benzenesulfonamide,
N-(1,3-benzodioxol-5-ylmethyl)-8-(3,4-dimethoxyphenyl)-2,7-dimethyl-pyrazolo[1,5-a][1,3,5]triazin-4-amine,
4-[[[8-(3,4-dimethoxyphenyl)-2,7-dimethyl-pyrazolo[1,5-a][1,3,5]triazin-4-yl]amino]methyl]phenol,
N-[4-[[[8-(3,4-dimethoxyphenyl)-2,7-dimethyl-pyrazolo[1,5-a][1,3,5]triazin-4-yl]amino]methyl]phenyl]methanesulfonamide,
N-benzyl-8-(3,4-dimethoxyphenyl)-2,7-dimethyl-pyrazolo[1,5-a][1,3,5]triazin-4-amine,
8-(3,4-dimethoxyphenyl)-2,7-dimethyl-N-(2-thienylmethyl)pyrazolo[1,5-a][1,3,5]triazin-4-amine,
8-(3,4-dimethoxyphenyl)-2,7-dimethyl-N-(3-pyridylmethyl)pyrazolo[1,5-a][1,3,5]triazin-4-amine,
8-(3,4-dimethoxyphenyl)-2,7-dimethyl-N-[(5-methyl-2-furyl)methyl]pyrazolo[1,5-a][1,3,5]triazin-4-amine,
methyl N-[4-[[[8-(3,4-dimethoxyphenyl)-2,7-dimethyl-pyrazolo[1,5-a][1,3,5]triazin-4-yl]amino]methyl]phenyl]sulfonylcarbamate,
N-[4-[[[8-(3,4-dimethoxyphenyl)-2,7-dimethyl-pyrazolo[1,5-a][1,3,5]triazin-4-yl]amino]methyl]phenyl]sulfonylpropanamide,
N-methyl-4-[[[8-(3,4-dimethoxyphenyl)-2,7-dimethyl-pyrazolo[1,5-a][1,3,5]triazin-4-yl]amino]methyl]benzenesulfonamide,
8-(3,4-dimethoxyphenyl)-2,7-dimethyl-N-[(1-methyl-1H-pyrazol-3-yl)methyl]pyrazolo[1,5-a][1,3,5]triazin-4-amine,
8-(3,4-dimethoxyphenyl)-2,7-dimethyl-N-[(1,3-oxazol-5-yl)methyl]pyrazolo[1,5-a][1,3,5]triazin-4-amine,
8-(3,4-dimethoxyphenyl)-2,7-dimethyl-N-[(2-methylpyrimidin-5-yl)methyl]pyrazolo[1,5-a][1,3,5]triazin-4-amine,
8-(3,4-dimethoxyphenyl)-2,7-dimethyl-N-[(pyridazin-4-yl)methyl]pyrazolo[1,5-a][1,3,5]triazin-4-amine,
8-(3,4-dimethoxyphenyl)-2,7-dimethyl-N-[(6-methylpyridin-3-yl)methyl]pyrazolo[1,5-a][1,3,5]triazin-4-amine,
3-(1,3-dimethyl-1H-indazol-5-yl)-2,6-dimethyl-N-(pyridin-4-ylmethyl)imidazo[1,2-b]pyridazin-8-amine,
3-(1,3-dimethyl-1H-indazol-5-yl)-2,6-dimethyl-N-((6-methylpyridin-3-yl)methyl)imidazo[1,2-b]pyridazin-8-amine,
N-(4-chlorobenzyl)-3-(1,3-dimethyl-1H-indazol-5-yl)-2,6-dimethylimidazo[1,2-b]pyridazin-8-amine,
3-(1,3-dimethyl-1H-indazol-5-yl)-N-((6-methoxypyridin-3-yl)methyl)-2,6-dimethylimidazo[1,2-b]pyridazin-8-amine,
3-(1,3-dimethyl-1H-indazol-5-yl)-2,6-dimethyl-N-(thiophen-2-ylmethyl)imidazo[1,2-b]pyridazin-8-amine,
3-(1,3-dimethyl-1H-indazol-5-yl)-N-(4-methoxybenzyl)-2,6-dimethylimidazo[1,2-b]pyridazin-8-amine,
3-(1,3-dimethyl-1H-indazol-5-yl)-2,6-dimethyl-N-(4-(methylsulfonyl)benzyl)imidazo[1,2-b]pyridazin-8-amine,
3-(1,3-dimethyl-1H-indazol-5-yl)-2,5-dimethyl-N-(pyridin-4-ylmethyl)pyrazolo[1,5-a]pyrimidin-7-amine,
3-(1,3-dimethyl-1H-indazol-5-yl)-2,5-dimethyl-N-((6-methylpyridin-3-yl)methyl)pyrazolo[1,5-a]pyrimidin-7-amine,
3-(1,3-dimethyl-1H-indazol-5-yl)-N-(4-methoxybenzyl)-2,5-dimethylpyrazolo[1,5-a]pyrimidin-7-amine,
N-(4-chlorobenzyl)-3-(1,3-dimethyl-1H-indazol-5-yl)-2,5-dimethylpyrazolo[1,5-a]pyrimidin-7-amine,
3-(1,3-dimethyl-1H-indazol-5-yl)-2,5-dimethyl-N-(4-(methylsulfonyl)benzyl)pyrazolo[1,5-a]pyrimidin-7-amine,
4-(((3-(1,3-dimethyl-1H-indazol-5-yl)-2,5-dimethylpyrazolo[1,5-a]pyrimidin-7-yl)amino)methyl)benzenesulfonamide,
3-(1,3-dimethyl-1H-indazol-5-yl)-N-((6-methoxypyridin-3-yl)methyl)-2,5-dimethylpyrazolo[1,5-a]pyrimidin-7-amine,
3-(1,3-dimethyl-1H-indazol-5-yl)-2,5-dimethyl-N-(thiophen-2-ylmethyl)pyrazolo[1,5-a]pyrimidin-7-amine,
N-(4-(((3-(1,3-dimethyl-1H-indazol-5-yl)-2,5-dimethylpyrazolo[1,5-a]pyrimidin-7-yl)amino)methyl)phenyl)methanesulfonamide,
3-(1,3-dimethyl-1H-indazol-5-yl)-2,5-dimethyl-N-((6-trifluoromethylpyridin-3-yl)methyl)pyrazolo[1,5-a]pyrimidin-7-amine,
N-(benzyl)-3-(1,3-dimethyl-1H-indazol-5-yl)-2,5-dimethylpyrazolo[1,5-a]pyrimidin-7-amine,
N-(4-fluorobenzyl)-3-(1,3-dimethyl-1H-indazol-5-yl)-2,5-dimethylpyrazolo[1,5-a]pyrimidin-7-amine,
8-(1,3-dimethyl-1H-indazol-5-yl)-2,7-dimethyl-N-(pyridin-4-ylmethyl)pyrazolo[1,5-a][1,3,5]triazin-4-amine,
8-(1,3-dimethyl-1H-indazol-5-yl)-N-(4-methoxybenzyl)-2,7-dimethylpyrazolo[1,5-a][1,3,5]triazin-4-amine,
8-(1,3-dimethyl-1H-indazol-5-yl)-2,7-dimethyl-N-((6-methylpyridin-3-yl)methyl)pyrazolo[1,5-a][1,3,5]triazin-4-amine,
4-(((8-(1,3-dimethyl-1H-indazol-5-yl)-2,7-dimethylpyrazolo[1,5-a][1,3,5]triazin-4-yl)amino)methyl)benzenesulfonamide,
N-(4-chlorobenzyl)-8-(1,3-dimethyl-1H-indazol-5-yl)-2,7-dimethylpyrazolo[1,5-a][1,3,5]triazin-4-amine,
8-(1,3-dimethyl-1H-indazol-5-yl)-2,7-dimethyl-N-(4-(methylsulfonyl)benzyl)pyrazolo[1,5-a][1,3,5]triazin-4-amine,
N-(4-(((8-(1,3-dimethyl-1H-indazol-5-yl)-2,7-dimethylpyrazolo[1,5-a][1,3,5]triazin-4-yl)amino)methyl)phenyl)methanesulfonamide,
8-(1,3-dimethyl-1H-indazol-5-yl)-2,7-dimethyl-N-(thiophen-2-ylmethyl)pyrazolo[1,5-a][1,3,5]triazin-4-amine, and
8-(1,3-dimethyl-1H-indazol-5-yl)-N-((6-methoxypyridin-3-yl)methyl)-2,7-dimethylpyrazolo[1,5-a][1,3,5]triazin-4-amine.

21. The method of claim 18, wherein the oral transmucosal administration, is buccal transmucosal administration.

22. The method of claim 18, wherein the virus is selected from viruses belonging to the Picornaviridae, Flaviviridae, Retroviridae, Filoviridae, Togaviridae, Papovaviridae, Papillomaviridae, Polyomaviridae, Caliciviridae, Coronaviridae, Hepeviridae, Bunyaviridae, Poxviridae or Orthomyxoviridae families.

23. The method of claim 22, wherein the virus is selected from viruses belonging to the Picornaviridae family, and viruses belonging to the Pneumoviridae family.

24. The method of claim 23, wherein the virus belonging to the Picornaviridae family is a virus belonging to the Enterovirus genus; and the virus belonging to the Pneumoviridae family is a virus belonging to the Orthopneumovirus genus.

25. The method of claim 18, wherein the oral transmucosal administration is buccal transmucosal administration.

26. The pharmaceutical formulation according to claim 7, wherein the compound of formula (I) or the pharmaceutically acceptable salt thereof is present in the formulation in an amount of from 5 to 40% by weight, relative to the total dry weight of the formulation.

27. The pharmaceutical formulation according to claim 7, wherein the compound of formula (I) or the pharmaceutically acceptable salt thereof is present in the formulation in an amount of from 10 to 30% by weight, relative to the total dry weight of the formulation.

28. The pharmaceutical formulation according to claim 7, wherein the formulation is a mucoadhesive oral film or a mucoadhesive buccal film.
